(19) United States
(12) Patent Application Publication
(10) Pub. No.: US 2026/0317347 A1
(43) Pub. Date: Oct. 15, 2026

(54) MACROCYCLIC COMPOUNDS AND THERAPEUTIC USES THEREOF

(71) Applicant: SUNRISE ONCOLOGY (HONG KONG) LIMITED, Hong Kong (CN)

(72) Inventors: Xiaotian TONG, Shanghai (CN); Wenbo ZHOU, Shanghai (CN); Huang CHEN, Shanghai (CN); Jian LU, Shanghai (CN); Shihong PENG, Shanghai (CN); Mingyao LIU, Shanghai (CN)

(73) Assignee: SUNRISE ONCOLOGY (HONG KONG) LIMITED, Hong Kong (CN)

(21) Appl. No.: 19/479,200

(22) PCT Filed: Apr. 26, 2024
(86) PCT No.: PCT/CN2024/090044

§ 371 Date: Oct. 27, 2025

(30) Foreign Application Priority Data
Apr. 27, 2023 (WO) PCT/CN2023/091355
Jul. 14, 2023 (WO) PCT/CN2023/107552

(51) Int. Cl. C07F 5/02; A61K 31/675; A61K 31/69; C07F 9/6561

(52) U.S. Cl. CPC C07F 5/025; A61K 31/675; A61K 31/69; C07F 9/6561

(57) ABSTRACT

Related to macrocyclic compounds and therapeutic uses thereof. Discloses a compound of formula (I), or a pharmaceutically acceptable salt thereof, a solvate thereof or a solvate of the pharmaceutically acceptable salt, and their use for the preparation of a medicament of treatment of cancer.

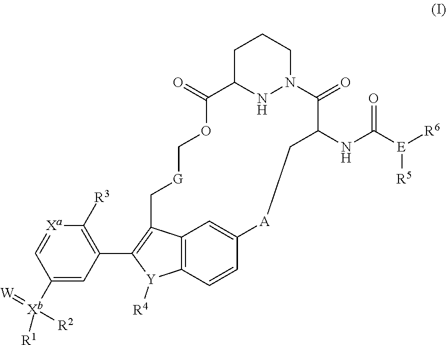

The present invention claims the priority of the PCT/CN2023/091355, filed on Apr. 27, 2023, the contents of which are incorporated herein by its entirety. The present invention claims the priority of the PCT/CN2023/107552, filed on Jul. 14, 2023, the contents of which are incorporated herein by its entirety.

### FIELD OF INVENTION

The present invention related to macrocyclic compounds and therapeutic uses thereof.

### PRIOR ARTS

The RAS protein (including KRAS4A, KRAS4B, HRAS, and NRAS) is a small, membrane-bound guanine nucleotide-binding protein that acts as a molecular switch by cycling between an active GTP-bound state and an inactive GDP-bound state. When RAS binds to GEF family proteins such as SOS1, its conformation changes, reducing its affinity for GDP and causing its dissociation from GDP and binding to GTP in the high concentration in the environment, thus becoming active. In normal cells, activation of receptor tyrosine kinases such as epidermal growth factor receptor (EGFR) promotes the exchange of GDP and GTP in RAS, activating RAS, which plays a key role in regulating cell survival, proliferation, and differentiation through multiple cascading reactions, including the PI3K-AKT-mTOR, RAF-MEK-ERK, and RALGDS-RAL pathways. Mutations in RAS are often driving forces for carcinogenesis and tumor development, as they disrupt the guanine exchange cycle and lock RAS in the activated state, continuously activating downstream signaling pathways and leading to the development of tumors.

RAS mutations occur in approximately 19% of all cancers and play an important role in tumor occurrence and progression. Among them, KRAS is the most commonly mutated subtype, followed by NRAS and HRAS. KRAS mutations commonly occur in pancreatic ductal adenocarcinoma, lung adenocarcinoma, and colorectal adenocarcinoma, while NRAS mutations have a relatively higher frequency in hematologic malignancies such as chronic myeloid leukemia and acute myeloid leukemia, as well as in malignant melanoma, thyroid cancer, and laryngeal cancer. HRAS mutations are less common in human cancers, but have a relatively higher mutation rate in head and neck squamous cell carcinoma, bladder cancer, salivary gland carcinoma, and oral cancer.

The RAS molecular glue can simultaneously bind to highly activated RAS (RAS-ON) proteins and a ubiquitously expressed protein in cells, forming a tricomplex to occupy binding sites for RAS interacting proteins such as SOS1 and RAF1, keeping RAS protein in a locked state alone, unable to activate downstream signaling pathways, thereby blocking RAS signaling pathway transmission and inhibiting tumor growth.

### Content of the Present Invention

Provided herein are Ras inhibitors as molecular glues. The approach described herein entails formation of a high affinity three-component complex between a synthetic ligand and two intracellular proteins which do not interact under normal physiological conditions: The target of interest (e.g. Ras), and a widely expressed cytosolic chaperone in cells (e.g. cyclophilin A). More specifically, in some embodiments, the Ras molecular glues described herein induce a new binding pocket in Ras by forming a three-component complex with high-affinity between the Ras protein and the cytosolic chaperone, cyclophilin A. Without being bound by theory, the inventors believe that one way the inhibitory effect on Ras is effected by compounds of the invention and the three-component complexes they form is by steric occlusion of the interaction site between Ras and downstream effector molecules, such as RAF and PI3K, which are required for propagating the oncogenic signal.

The present invention discloses a compound of formula (I), or a pharmaceutically acceptable salt thereof, a solvate thereof or a solvate of the pharmaceutically acceptable salt,

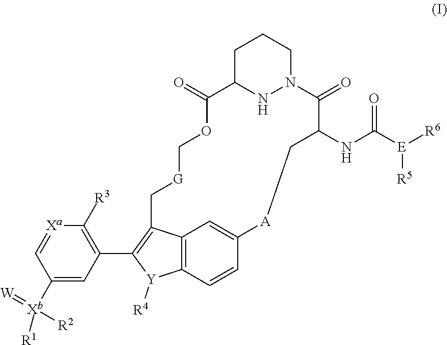

Wherein, A is 5-6 membered heterocycloalkylene, 5-6 membered heteroarylene, phenylene or phenylene optionally substituted by hydroxy or boric acid group;
G is C₁₋₆ alkylene;
X^{a} is CH or N;
X^{b} is P or B;
Y is O, S or N;
W is absent, O or S, provided that when X^{b} is B, W is absent;
R¹ and R² are each independently selected from hydroxy, C₁₋₆ alkyl, C₁₋₆ alkoxyl, C₁₋₆ haloalkyl, C₁₋₆ haloalkoxyl, or R¹ and R² combine with the atoms to which they are attached to form a 4 to 10-membered ring, wherein the ring is selected from cycloalkyl optionally substituted by halogen, C₁₋₆ alkyl, C₁₋₆ haloalkyl, C₁₋₆ haloalkoxy; heterocycloalkyl optionally substituted by halogen, C₁₋₆ alkyl, C₁₋₆haloalkyl, C₁₋₆ haloalkoxy;
R³ is C₁₋₆ alkoxyC₁₋₆ alkyl;
R⁴ is absent, or selected from hydrogen, C₁₋₆ alkyl, or C₁₋₆ haloalkyl, provided that when Y is O or S, R⁴ is absent;
E is a bond, or selected from N, or —CR^{A}—, wherein R^{A} is selected from halogen, hydroxy, C₁₋₆ alkyl, C₁₋₆ haloalkyl, C₁₋₆ alkoxyl, C₁₋₆ AlkoxyC₁₋₆ alkyl, —NR^{B}R^{C}, cyano, C₁₋₆ aminoalkyl, C₁₋₆ hydroxyalkyl, or carboxyl; wherein R^{B} and R^{C} are independently selected from hydrogen, or C₁₋₆ alkyl;
R⁵ is hydrogen, C₁₋₆ alkyl, C₁₋₆ heteroalkyl, C₃₋₁₀ heterocycloalkyl, C₃₋₁₀ cycloalkyl, C₆₋₁₀ aryl, C₅₋₁₀ heteroaryl, or

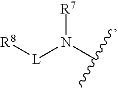

wherein R⁷ is hydrogen, or C₁₋₆ alkyl;
R⁸ is C₁₋₆ alkyl, C₁₋₆heteroalkyl, C₃₋₁₀ heterocycloalkyl, C₃₋₁₀ cycloalkyl, C₆₋₁₀ aryl, C₅₋₁₀ heteroaryl;
L is absent, or selected from —CH₂—, —C(O)—, —CHR⁸— or —C(R⁸)₂—, wherein R⁸ is C₁₋₆alkyl;
R⁶ is hydrogen, C₁₋₆ alkyl or C₃₋₆ cycloalkyl;
or R⁵ and R⁶ combine with the atoms to which they are attached to form a ring, wherein the ring is selected from 3- to 10-membered cycloalkyl optionally substituted by at least one of halogen, C₁₋₆ alkyl, C₁₋₆ haloalkyl, C₁₋₆ alkoxyl, C₁₋₆ haloalkoxyl, C₁₋₆ hydroxyalkyl, C₁₋₆ alkoxyC₁₋₆alkyl, or oxo; 3- to 10-membered heterocycloalkyl optionally substituted by at least one of halogen, hydroxyl, C₁₋₆ alkyl, C₁₋₆ alkoxyl, C₁₋₆ haloalkyl, C₁₋₆ haloalkoxyl, C₁₋₆ alkoxyC₁₋₆alkyl, C₁₋₆haloalkoxyC₁₋₆ alkyl, C₆₋₁₀aryl, C₅₋₁₀ heteroaryl, C(O)C₁₋₆ alkyl, C(O)OC₁₋₆ alky, or oxo; or R^{A}, R⁵ and R⁶ combine with the atoms to which they are attached to form a bicyclo C₇₋₁₄ heterocycloalkyl.

Another embodiment present invention is a compound of formula (Ia) or (Ib), or a pharmaceutically acceptable salt thereof, a solvate thereof or a solvate of the pharmaceutically acceptable salt,

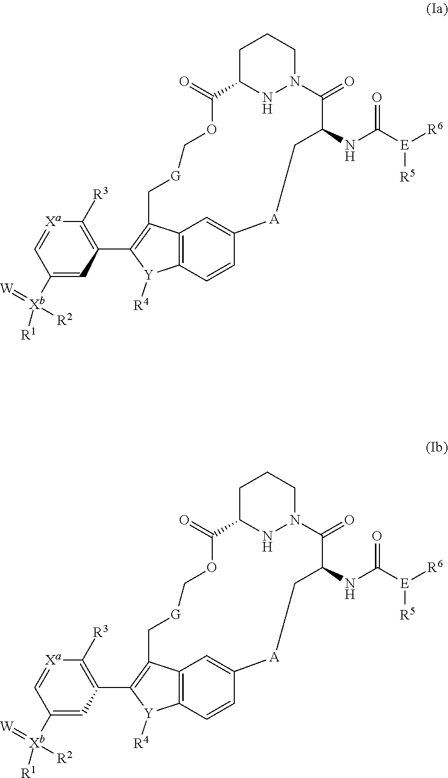

Wherein, A is 5-6 membered heterocycloalkylene, 5-6 membered heteroarylene, phenylene or phenylene optionally substituted by hydroxy or boric acid group;
G is C₁₋₆alkylene;
X^{a} is CH or N;
X^{b} is P or B;
Y is O, S or N;
W is absent, O or S, provided that when X^{b} is B, W is absent;
R¹ and R² are each independently selected from hydroxy, C₁₋₆ alkyl, C₁₋₆ alkoxyl, C₁₋₆ haloalkyl, C₁₋₆ haloalkoxyl, or R¹ and R² combine with the atoms to which they are attached to form a 4 to 10-membered ring, wherein the ring is selected from cycloalkyl optionally substituted by halogen, C₁₋₆ alkyl, C₁₋₆ haloalkyl, C₁₋₆ haloalkoxy; heterocycloalkyl optionally substituted by halogen, C₁₋₆ alkyl, C₁₋₆ haloalkyl, C₁₋₆ haloalkoxy;
R³ is C₁₋₆ alkoxyC₁₋₆ alkyl;
R⁴ is absent, or selected from hydrogen, C₁₋₆ alkyl, or C₁₋₆ haloalkyl, provided that when Y is O or S, R⁴ is absent;
E is a bond, or selected from N, or —CR^{A}—, wherein R^{A} is selected from halogen, hydroxy, C₁₋₆ alkyl, C₁₋₆ haloalkyl, C₁₋₆ alkoxyl, C₁₋₆ AlkoxyC₁₋₆ alkyl, —NR^{B}R^{C}, cyano, C₁₋₆ aminoalkyl, C₁₋₆ hydroxyalkyl, or carboxyl; wherein R^{B} and R^{C} are independently selected from hydrogen, or C₁₋₆ alkyl;
R⁵ is hydrogen, C₁₋₆ alkyl, C₁₋₆ heteroalkyl, C₃₋₁₀ heterocycloalkyl, C₃₋₁₀ cycloalkyl, C₆₋₁₀ aryl, C₅₋₁₀ heteroaryl, or

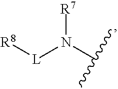

wherein R⁷ is hydrogen, or C₁₋₆ alkyl;
R⁸ is C₁₋₆ alkyl, C₁₋₆ heteroalkyl, C₃₋₁₀ heterocycloalkyl, C₃₋₁₀ cycloalkyl, C₆₋₁₀ aryl, C₅₋₁₀ heteroaryl;
L is absent, or selected from —CH₂—, —C(O)—, —CHR⁸— or —C(R⁸)₂—, wherein R⁸ is C₁₋₆ alkyl;
R⁶ is hydrogen, C₁₋₆alkyl or C₃₋₆cycloalkyl;
or R⁵ and R⁶ combine with the atoms to which they are attached to form a ring, wherein the ring is selected from 3- to 10-membered cycloalkyl optionally substituted by at least one of halogen, C₁₋₆ alkyl, C₁₋₆ haloalkyl, C₁₋₆ alkoxyl, C₁₋₆ haloalkoxyl, C₁₋₆ hydroxyalkyl, C₁₋₆ alkoxyC₁₋₆alkyl, or oxo; 3- to 10-membered heterocycloalkyl optionally substituted by at least one of halogen, hydroxyl, C₁₋₆alkyl, C₁₋₆alkoxyl, C₁₋₆ haloalkyl, C₁₋₆ haloalkoxyl, C₁₋₆ alkoxyC₁₋₆alkyl, C₁₋₆ haloalkoxyC₁₋₆alkyl, C₆₋₁₀aryl, C₅₋₁₀ heteroaryl, C(O)C₁₋₆ alkyl, C(O)OC₁₋₆ alky, or oxo; or R^{A}, R⁵ and R⁶ combine with the atoms to which they are attached to form a bicyclo C₇₋₁₄ heterocycloalkyl.

The present invention discloses a compound of formula (I), or a pharmaceutically acceptable salt thereof, a solvate thereof or a solvate of the pharmaceutically acceptable salt,

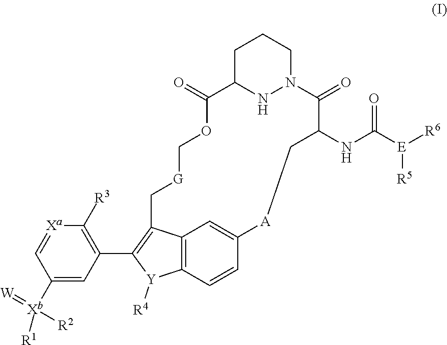

wherein
A is 5-6 membered heterocycloalkylene, 5-6 membered heteroarylene, phenylene, or the phenylene optionally substituted by hydroxy or boric acid group;
G is C₁₋₆ alkylene;
X^{a} is CH or N;
X^{b} is P or B;
Y is O, S or N;
W is absent, O or S, provided that when X^{b} is B, W is absent;
R¹ and R² are each independently selected from hydroxy, C₁₋₆ alkyl, C₁₋₆ alkoxyl, C₁₋₆ haloalkyl, C₁₋₆ haloalkoxyl, or R¹ and R² combine with the atoms to which they are attached to form a 4 to 10-membered ring, wherein the ring is heterocycloalkyl; and the heterocycloalkyl optionally substituted by halogen, C₁₋₆ alkyl, C₁₋₆ haloalkyl, C₁₋₆ haloalkoxy;
R³ is C₁₋₆alkoxyC₁₋₆alkyl;
R⁴ is absent, or selected from hydrogen, C₁₋₆ alkyl, or C₁₋₆ haloalkyl, provided that when Y is O or S, R⁴ is absent;
E is a bond, or selected from N, or —CR^{A}—, wherein R^{A} is selected from hydrogen, halogen, hydroxy, C₁₋₆ alkyl, C₁₋₆ haloalkyl, C₁₋₆ alkoxyl, C₁₋₆alkyl substituted by C₁₋₆ alkoxy, —NR^{B}R^{C}, cyano, C₁₋₆ aminoalkyl, C₁₋₆ hydroxyalkyl, or carboxyl; wherein R^{B} and R^{C} are independently selected from hydrogen, or C₁₋₆ alkyl;
R⁵ is hydrogen, C₁₋₆ alkyl, C₁₋₆ heteroalkyl, C₃₋₁₀ heterocycloalkyl, C₃₋₁₀ cycloalkyl, C₆₋₁₀ aryl, C₅₋₁₀ heteroaryl, or

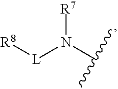

wherein R⁷ is hydrogen, or C₁₋₆ alkyl;
R⁸ is C₁₋₆ alkyl, C₁₋₆ heteroalkyl, C₃₋₁₀ heterocycloalkyl, C₃₋₁₀ cycloalkyl, C₆₋₁₀ aryl, C₅₋₁₀ heteroaryl; the C₃₋₁₀ heterocycloalkyl optionally substituted by C₁₋₆ alkyl, C₆₋₁₀ aryl, —CO—O—C₃₋₆ cycloalkyl or —CO—O—C₁₋₆ alkyl; the C₃₋₁₀ cycloalkyl and C₃₋₆ cycloalkyl optionally substituted by hydroxyl, cyano, halogen, or C₁₋₆ alkyl;
L is absent, or selected from —CH₂—, —C(O)—, —CHR⁹— or —C(R⁹)₂—, wherein R⁹ is C₁₋₆ alkyl;
R⁶ is hydrogen, C₁₋₆ alkyl or C₃₋₆ cycloalkyl;
or R⁵ and R⁶ combine with the atoms to which they are attached to form a ring, wherein the ring is selected from 3- to 10-membered cycloalkyl and 3- to 10-membered heterocycloalkyl; the 3- to 10-membered cycloalkyl optionally substituted by at least one of halogen, C₁₋₆ alkyl, C₁₋₆ haloalkyl, C₁₋₆ alkoxyl, C₁₋₆ haloalkoxyl, C₁₋₆ hydroxyalkyl, C₁₋₆ alkoxyC₁₋₆ alkyl, or oxo; the 3- to 10-membered heterocycloalkyl optionally substituted by at least one of halogen, hydroxyl, C₁₋₆ alkyl, C₁₋₆ alkoxyl, C₁₋₆ haloalkyl, C₁₋₆ haloalkoxyl, —C₁₋₆ alkoxyC₁₋₆ alkyl, C₁₋₆ haloalkoxyC₁₋₆ alkyl, C₆₋₁₀ aryl, C₅₋₁₀ heteroaryl, C(O)C₁₋₆ alkyl, C(O)OC₁₋₆ alky, or oxo; or R^{A}, R⁵ and R⁶ combine with the atoms to which they are attached to form a bicyclo C₇₋₁₄ heterocycloalkyl;
in the 5-6 membered heterocycloalkylene, 5-6 membered heteroarylene, heterocycloalkyl, C₁₋₆ heteroalkyl, C₃₋₁₀ heterocycloalkyl, C₅₋₁₀ heteroaryl, bicyclo C₇₋₁₄ heterocycloalkyl and 3- to 10-membered heterocycloalkyl, the heteroatom is independently selected from one or more of N, O and S, and the number of the heteroatom is independently 1-3.

In some embodiments of the present disclosure, the compound of formula (I) is a compound of formula (Ia) or (Ib),

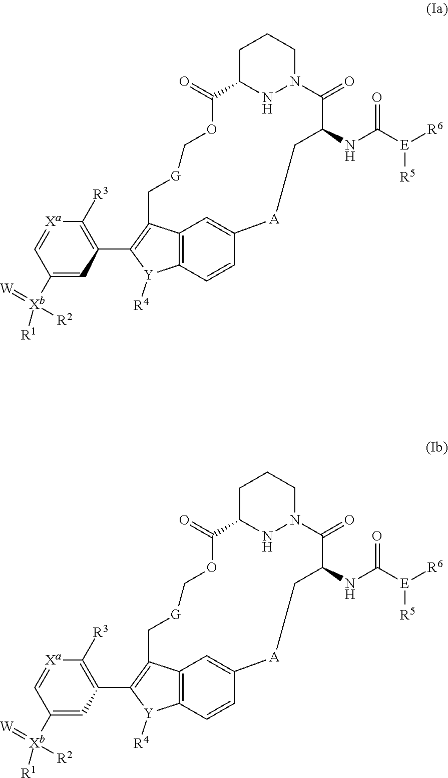

wherein
A is 5-6 membered heterocycloalkylene, 5-6 membered heteroarylene, phenylene, or the phenylene optionally substituted by hydroxy or boric acid group;
G is C₁₋₆ alkylene;
X^{a} is CH or N;
X^{b} is P or B;
Y is O, S or N;
W is absent, O or S, provided that when X^{b} is B, W is absent;
R¹ and R² are each independently selected from hydroxy, C₁₋₆ alkyl, C₁₋₆ alkoxyl, C₁₋₆ haloalkyl, C₁₋₆ haloalkoxyl, or R¹ and R² combine with the atoms to which they are attached to form a 4 to 10-membered ring, wherein the ring is heterocycloalkyl; and the heterocycloalkyl optionally substituted by halogen, C₁₋₆ alkyl, C₁₋₆haloalkyl, C₁₋₆ haloalkoxy;
R³ is or C₁₋₆ alkyl substituted by C₁₋₆alkoxy;
R⁴ is absent, or selected from hydrogen, C₁₋₆ alkyl, or C₁₋₆ haloalkyl, provided that when Y is O or S, R⁴ is absent;
E is a bond, or selected from N, or —CR^{A}—, wherein R^{A} is selected from hydrogen, halogen, hydroxy, C₁₋₆ alkyl, C₁₋₆ haloalkyl, C₁₋₆ alkoxyl, C₁₋₆ alkyl substituted by C₁₋₆ alkoxy, —NR^{B}R^{C}, cyano, C₁₋₆ aminoalkyl, C₁₋₆ hydroxyalkyl, or carboxyl; wherein R^{B} and R^{C} are independently selected from hydrogen, or C₁₋₆ alkyl;
R⁵ is hydrogen, C₁₋₆ alkyl, C₁₋₆ heteroalkyl, C₃₋₁₀ heterocycloalkyl, C₃₋₁₀ cycloalkyl, C₆₋₁₀ aryl, C₅₋₁₀ heteroaryl, or

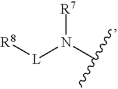

wherein R⁷ is hydrogen, or C₁₋₆ alkyl;
R⁸ is C₁₋₆ alkyl, C₁₋₆ heteroalkyl, C₃₋₁₀ heterocycloalkyl, C₃₋₁₀ cycloalkyl, C₆₋₁₀ aryl, C₅₋₁₀ heteroaryl; the C₃₋₁₀heterocycloalkyl optionally substituted by C₁₋₆ alkyl, C₆₋₁₀ aryl, —CO—O—C₃₋₆ cycloalkyl or —CO—O—C₁₋₆ alkyl; the C₃₋₁₀cycloalkyl and C₃₋₆ cycloalkyl optionally substituted by hydroxyl, cyano, halogen, or C₁₋₆ alkyl;
L is absent, or selected from —CH₂—, —C(O)—, —CHR⁹— or —C(R⁹)₂—, wherein R⁹is C₁₋₆ alkyl;
R⁶ is hydrogen, C₁₋₆ alkyl or C₃₋₆ cycloalkyl;
or R⁵ and R⁶ combine with the atoms to which they are attached to form a ring, wherein the ring is selected from 3- to 10-membered cycloalkyl and 3- to 10-membered heterocycloalkyl; the 3- to 10-membered cycloalkyl optionally substituted by at least one of halogen, C₁₋₆ alkyl, C₁₋₆ haloalkyl, C₁₋₆ alkoxyl, C₁₋₆ haloalkoxyl, C₁₋₆ hydroxyalkyl, C₁₋₆ alkoxyC₁₋₆ alkyl, or oxo; the 3- to 10-membered heterocycloalkyl optionally substituted by at least one of halogen, hydroxyl, C₁₋₆ alkyl, C₁₋₆ alkoxyl, C₁₋₆ haloalkyl, C₁₋₆ haloalkoxyl, C₁₋₆ alkoxyC₁₋₆ alkyl, C₁₋₆ haloalkoxyC₁₋₆ alkyl, C₆₋₁₀ aryl, C₅₋₁₀ heteroaryl, C(O)C₁₋₆ alkyl, C(O)OC₁₋₆ alky, or oxo;
or R^{A}, R⁵ and R⁶ combine with the atoms to which they are attached to form a bicyclo C₇₋₁₄ heterocycloalkyl;
in the 5-6 membered heterocycloalkylene, 5-6 membered heteroarylene, heterocycloalkyl, C₁₋₆heteroalkyl, C₃₋₁₀ heterocycloalkyl, C₅₋₁₀ heteroaryl, bicyclo C₇₋₁₄ heterocycloalkyl, and 3- to 10-membered heterocycloalkyl, the heteroatom is independently selected from one or more of N, O and S, and the number of the heteroatom is independently 1-3.

In some embodiments of the present disclosure,
wherein A is 5-6 membered heterocycloalkylene, 5-6 membered heteroarylene, phenylene, or the phenylene optionally substituted by hydroxy or boric acid group;
G is C₁₋₆alkylene;
X^{a} is CH or N;
X^{b} is P or B;
Y is N;
W is absent, O or S, provided that when X^{b} is B, W is absent;
R¹ and R² are each independently selected from hydroxy, C₁₋₆ alkyl, or R¹ and R² combine with the atoms to which they are attached to form a 4 to 10-membered ring, wherein the ring is heterocycloalkyl; the heterocycloalkyl optionally substituted by C₁₋₆ alkyl;
R³ is C₁₋₆ alkyl substituted by C₁₋₆ alkoxy;
R⁴ is hydrogen, C₁₋₆ alkyl, or C₁₋₆ haloalkyl;
E is a bond, or selected from N, or —CR^{A}—, wherein R^{A} is selected from hydrogen, halogen, hydroxyC₁₋₆ alkyl, C₁₋₆ haloalkyl, C₁₋₆ alkoxyl, C₁₋₆alkyl substituted by C₁₋₆ alkoxy, C₁₋₆ alkoxy substituted by C₁₋₆ alkyl, —NR^{B}R^{C}, cyano, C₁₋₆ aminoalkyl, C₁₋₆ hydroxyalkyl, or carboxyl; wherein R^{B} and R^{C} are independently selected from hydrogen, or C₁₋₆ alkyl;
R⁵ is hydrogen, C₁₋₆ alkyl, 3- to 10-membered heterocycloalkyl, C₃₋₁₀ cycloalkyl, C₆₋₁₀ aryl, C₅₋₁₀ heteroaryl, or

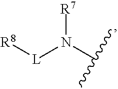

wherein R⁷ is hydrogen, or C₁₋₆ alkyl;
R⁸ is C₁₋₆ alkyl, 3- to 10-membered heterocycloalkyl; the 3- to 10-membered heterocycloalkyl optionally substituted by C₁₋₆ alkyl, C₆₋₁₀ aryl, —CO—O—C₃₋₆ cycloalkyl or —CO—O—C₁₋₆ alkyl; the C₃₋₁₀ cycloalkyl optionally substituted by hydroxyl, cyano, halogen, or C₁₋₆ alkyl;
L is absent, or selected from —CH₂—, —C(O)—, —CHR⁹— or —C(R⁹)₂—, wherein R⁹ is C₁₋₆ alkyl;
R⁶ is hydrogen, C₁₋₆ alkyl or C₃₋₆ cycloalkyl;
or R⁵ and R⁶ combine with the atoms to which they are attached to form a ring, wherein the ring is selected from 3- to 10-membered cycloalkyl and 3- to 10-membered heterocycloalkyl; the 3- to 10-membered cycloalkyl optionally substituted by at least one of halogen C₁₋₆ alkyl, C₁₋₆ haloalkyl, C₁₋₆ alkoxyl, C₁₋₆ haloalkoxyl, C₁₋₆ hydroxyalkyl, C₁₋₆ alkoxyC₁₋₆alkyl, or oxo; the 3- to 10-membered heterocycloalkyl optionally substituted by at least one of halogen, hydroxyl, C₁₋₆ alkyl, C₁₋₆ alkoxyl, C₁₋₆ haloalkyl, C₁₋₆ haloalkoxyl, —NR^{D}R^{E}, C₁₋₆ alkoxyC₁₋₆alkyl, C₁₋₆haloalkoxyC₁₋₆alkyl, C₆₋₁₀aryl, C₅₋₁₀ heteroaryl, C(O)C₁₋₆ alkyl, C(O)OC₁₋₆ alky, or oxo;
in the 5-6 membered heterocycloalkylene, 5-6 membered heteroarylene, heterocycloalkyl, and 3- to 10-membered heterocycloalkyl, the heteroatom is independently selected from one or more of N, O and S, and the number of the heteroatom is independently 1-3.

In some embodiments of the present disclosure, when X^{b} is B

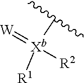

is

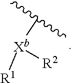

In some embodiments of the present disclosure, wherein A is 5-6 membered heterocycloalkylene, phenylene or phenylene optionally substituted by hydroxy or boric acid group;
G is C₁₋₆ alkylene;
X^{a} is CH;
X^{b} is P or B;
Y is N;
W is absent, O or S, provided that when X^{b} is B, W is absent;
R¹ and R² are each independently selected from hydroxy, and C₁₋₆ alkyl,
R³ is C₁₋₆ alkoxy substituted by C₁₋₆ alkyl;
R⁴ is hydrogen or C₁₋₆ alky;
E is a bond, or selected from N, or —CR^{A}—, wherein R^{A} is selected from hydrogen, halogen, hydroxy or C₁₋₆ alkyl;
R⁵ is hydrogen, C₁₋₆ alkyl, 3- to 10-membered heterocycloalkyl, C₃₋₁₀ cycloalkyl, C₆₋₁₀ aryl, C₅₋₁₀ heteroaryl, or

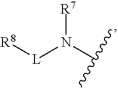

wherein R⁷ is hydrogen, or C₁₋₆ alkyl;
R⁸ is C₁₋₆ alkyl, 3- to 10-membered heterocycloalkyl; the 3- to 10-membered heterocycloalkyl optionally substituted by C₁₋₆ alkyl, C₆₋₁₀ aryl, —CO—O—C₁₋₆ cycloalkyl or —CO—O—C₁₋₆ alkyl; the C₃₋₁₀ cycloalkyl optionally substituted by hydroxyl, cyano, halogen, or C₁₋₆ alkyl;
L is absent, or selected from —CH₂—, —C(O)—, —CHR⁹— or —C(R⁹)₂—, wherein R⁹ is C₁₋₆alkyl;
R⁶ is hydrogen, C₁₋₆ alkyl or C₃₋₆ cycloalkyl;
or R⁵ and R⁶ combine with the atoms to which they are attached to form a ring, wherein the ring is selected from 3- to 10-membered cycloalkyl and 3- to 10-membered heterocycloalkyl; the 3- to 10-membered cycloalkyl optionally substituted by at least one of halogen C₁₋₆ alkyl, C₁₋₆ haloalkyl, C₁₋₆ alkoxyl, C₁₋₆ haloalkoxyl, C₁₋₆ hydroxyalkyl, C₁₋₆ alkoxyC₁₋₆alkyl, or oxo; the 3- to 10-membered heterocycloalkyl optionally substituted by at least one of halogen, hydroxyl, C₁₋₆ alkyl, C₁₋₆ alkoxyl, C₁₋₆ haloalkyl, C₁₋₆ haloalkoxyl, —NR^{D}R^{E}, C₁₋₆alkoxyC₁₋₆alkyl, C₁₋₆haloalkoxyC₁₋₆alkyl, C₆₋₁₀aryl, C₅₋₁₀heteroaryl, C(O)C₁₋₆alkyl, C(O)OC₁₋₆ alky, or oxo;
in 3- to 10-membered heterocycloalkyl, the heteroatom is independently selected from one or two of N and 0, and the number of the heteroatom is independently 1-2;
in 3- to 6-membered heterocycloalkyl, the heteroatom is independently selected from one or two of N and O, and the number of the heteroatom is independently 1-2;
in 5-6 membered heterocycloalkylene, the heteroatom is independently selected from one or two of N and S, and the number of the heteroatom is independently 1-2.

In some embodiments of the present disclosure, wherein A is 5-6 membered heterocycloalkylene, phenylene or phenylene optionally substituted by hydroxy or boric acid group;
G is C₁₋₆ alkylene;
X^{a} is CH;
X^{b} is P or B;
Y is N;
W is absent, O or S, provided that when X^{b} is B, W is absent;
R¹ and R² are each independently selected from hydroxy, and C₁₋₆ alkyl,
R³ is C₁₋₆ alkoxy substituted by C₁₋₆ alkyl;
R⁴ is hydrogen or C₁₋₆ alky;
E is a bond, or selected from N, or —CR^{A}—, wherein R^{A} is selected from hydrogen, halogen, hydroxy or C₁₋₆ alkyl;
R⁵ is hydrogen, C₁₋₆ alkyl, or

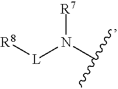

wherein R⁷ is hydrogen, or C₁₋₆ alkyl;
R⁸ is C₁₋₆ alkyl, 3- to 6-membered heterocycloalkyl; the 3- to 6-membered heterocycloalkyl optionally substituted by C₆₋₁₀ aryl or —CO—O—C₁₋₆ alkyl;
L is absent, or —C(O)—,
R⁶ is hydrogen, C₁₋₆alkyl or C₃₋₆ cycloalkyl;
in 3- to 6-membered heterocycloalkyl, the heteroatom is independently selected from one or two of N and O, and the number of the heteroatom is independently 1-2;
in 5-6 membered heterocycloalkylene, the heteroatom is independently selected from one or two of N and S, and the number of the heteroatom is independently 1-2.

In some embodiments of the present disclosure, in A, in the 5-6 membered heterocycloalkylene, the heteroatom is independently selected from one or two of N, O and S, and the number of the heteroatom is independently 1-2.

In some embodiments of the present disclosure, in A, in 5-6 membered heteroarylene, the heteroatom is independently selected from one or two of N, O and S, and the number of the heteroatom is independently 1-2, such as

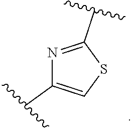 [SMILES: *c1csc(*)n1]

In some embodiments of the present disclosure, in R¹ and R², the C₁₋₆ alkyl is methyl, ethyl, propyl or isopropyl.

In some embodiments of the present disclosure, in R¹ and R², the C₁₋₆ alkoxyl is methoxy, ethoxy, n-propoxy or isopropoxy.

In some embodiments of the present disclosure, in R¹ and R², the C₁₋₆ haloalkyl is —CF₃.

In some embodiments of the present disclosure, in R¹ and R², the C₁₋₆ haloalkoxyl is —O—CF₃.

In some embodiments of the present disclosure, in R¹ and R², the 5 to 6-membered ring is heterocycloalkyl, the heteroatom is independently selected from one or two of N and P, and the number of the heteroatom is independently 1-2, preferably,

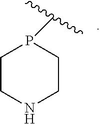 [SMILES: *P1CCNCC1]

In some embodiments of the present disclosure, in R³, the C₁₋₆alkoxyC₁₋₆alkyl is

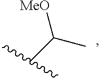 [SMILES: *C(C)OC]

preferably,

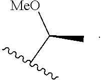 [SMILES: *[C@H](C)OC]

In some embodiments of the present disclosure, in R⁴, the C₁₋₆ alkyl is methyl, ethyl, propyl or isopropyl.

In some embodiments of the present disclosure, in R⁴, the C₁₋₆ haloalky is —CF₃, or —CH₂CF₃.

In some embodiments of the present disclosure, in R^{A}, the halogen is F, Cl, Br or I; preferably, F.

In some embodiments of the present disclosure, in R^{A}, the C₁₋₆ alkyl is methyl, ethyl, propyl or isopropyl.

In some embodiments of the present disclosure, in R^{A}, the C₁₋₆ haloalkyl is-CF₃.

In some embodiments of the present disclosure, in R^{A}, the C₁₋₆ alkoxyl is -o-methyl, -o-ethyl, -o-propyl or -o-isopropyl.

In some embodiments of the present disclosure, in R^{A}, the C₁₋₆alkyl is methyl, ethyl, propyl or isopropyl.

In some embodiments of the present disclosure, in R^{A}, the C₁₋₆ aminoalkyl is —NH-methyl, —NH-ethyl, —NH-propyl or —NH— isopropyl.

In some embodiments of the present disclosure, in R^{A}, the C₁₋₆ hydroxyalkyl is ydroxy-substituted methyl, hydroxy-substituted ethyl, hydroxy-substituted n-propyl or hydroxy-substituted isopropyl.

In some embodiments of the present disclosure, in R^{B} and R^{C}, the C₁₋₆ alkyl is methyl, ethyl, propyl or isopropyl.

In some embodiments of the present disclosure, in R⁵, the C₁₋₆ alkyl is methyl, ethyl, propyl or isopropyl.

In some embodiments of the present disclosure, in R⁵, in C₁₋₆ heteroalkyl, the heteroatom is N, O or S, and the number of the heteroatom is independently 1-2.

In some embodiments of the present disclosure, in R⁵, the 3- to 10-membered heterocycloalkyl is 3- to 7-membered heterocycloalkyl, preferably, the heteroatom is N, O or S, and the number of the heteroatom is independently 1-2.

In some embodiments of the present disclosure, in R⁵, the C₃₋₁₀ cycloalkyl is C₃₋₆ cycloalkyl, preferably, cyclopropyl, cyclobutyl, cyclopentyl or cyclohexyl.

In some embodiments of the present disclosure, in R⁵, the C₆₋₁₀ aryl is

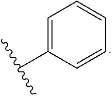 [SMILES: *c1ccccc1]

In some embodiments of the present disclosure, in R⁸, the C₁₋₆ alkyl is methyl, ethyl, propyl or isopropyl.

In some embodiments of the present disclosure, in R⁸, in C₁₋₆ heteroalkyl, the heteroatom is N, O or S, and the number of the heteroatom is independently 1-2.

In some embodiments of the present disclosure, in R⁸, the C₃₋₁₀ heterocycloalkyl is 3- to 6-membered heterocycloalkyl, preferably, the heteroatom is N, O or S, and the number of the heteroatom is independently 1-2, preferably,

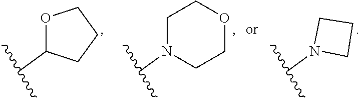 [SMILES: *C1CCCO1 ; *N1CCOCC1 ; *N1CCC1]

In some embodiments of the present disclosure, in R⁸, the C₃₋₁₀ cycloalky is C₃₋₆ cycloalkyl, preferably, cyclopropyl, cyclobutyl, cyclopentyl or cyclohexyl.

In some embodiments of the present disclosure, in R¹, the C₆₋₁₀ aryl is

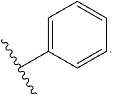 [SMILES: *c1ccccc1]

In some embodiments of the present disclosure, in R⁸, the —CO—O—C₁₋₆ alkyl is —CO—O-methyl, —CO—O-ethyl, —CO—O-propyl or —CO—O-isopropyl.

In some embodiments of the present disclosure, in R⁶, the C₁₋₆ alkyl is methyl, ethyl, propyl or isopropyl.

In some embodiments of the present disclosure, in R⁶, the C₃₋₆ cycloalkyl is cyclopropyl, cyclobutyl, cyclopentyl or cyclohexyl.

In some embodiments of the present disclosure, in G, the C₁₋₆ alkylene is methylene, ethylene, propylene or isopropylidene.

In some embodiments of the present disclosure, in bicyclo C₇₋₁₄ heterocycloalkyl, the heteroatom is N, O or S, and the number of the heteroatom is independently 1-2, preferably,

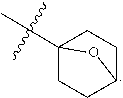 [SMILES: *C12CCC(CC1)O2]

In some embodiments of the present disclosure, A is 5-6 membered heterocycloalkylene, phenylene or phenylene optionally substituted by hydroxy or boric acid group; in the 5-6 membered heterocycloalkylene, the heteroatom is independently selected from one or more of N, O and S, and the number of the heteroatom is independently 1-3.

In some embodiments of the present disclosure, G is C₁₋₆ alkylene.

In some embodiments of the present disclosure, X^{a} is CH.

In some embodiments of the present disclosure, R¹ and R² are each independently selected from hydroxy, C₁₋₆ alkyl, or R¹ and R² combine with the atoms to which they are attached to form a 4 to 10-membered ring, wherein the ring is heterocycloalkyl; the heterocycloalkyl optionally substituted by C₁₋₆ alkyl, in the 4 to 10-membered ring, the heteroatom is independently selected from one or more of N, O and S, and the number of the heteroatom is independently 1-3; preferably, R¹ and R² are each independently selected from hydroxy and C₁₋₆ alkyl.

In some embodiments of the present disclosure, R³ is C₁₋₆ alkyl substituted by C₁₋₆ alkoxy.

In some embodiments of the present disclosure, R⁴ is hydrogen, C₁₋₆ alkyl, or C₁₋₆haloalkyl; preferably, R⁴ is hydrogen or C₁₋₆ alky.

In some embodiments of the present disclosure, E is a bond, or selected from N, or —CR^{A}—, wherein R^{A} is selected from hydrogen, halogen, hydroxyC₁₋₆ alkyl, C₁₋₆haloalkyl, C₁₋₆alkoxyl, C₁₋₆alkyl substituted by C₁₋₆ alkoxy, C₁₋₆alkoxy substituted by C₁₋₆alkyl, —NR^{B}R^{C}, cyano, C₁₋₆aminoalkyl, C₁₋₆hydroxyalkyl, or carboxyl: wherein R^{B} and R^{C} are independently selected from hydrogen, or C₁₋₆alkyl; preferably, E is a bond, or selected from N, or —CR^{A}—, wherein R^{A} is selected from hydrogen, halogen, hydroxy or C₁₋₆ alkyl.

In some embodiments of the present disclosure, R⁵ is hydrogen, C₁₋₆ alkyl, 3- to 10-membered heterocycloalkyl, C₃₋₁₀ cycloalkyl, C₆₋₁₀aryl, C₅₋₁₀ heteroaryl, or

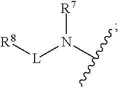

wherein R⁷ is hydrogen, or C₁₋₆ alkyl; R¹ is C₁₋₆alkyl, 3- to 10-membered heterocycloalkyl; the 3- to 10-membered heterocycloalkyl optionally substituted by C₁₋₆ alkyl, C₆₋₁₀ aryl, —CO—O—C₃₋₆ cycloalkyl or —CO—O—C₁₋₆ alkyl; the C₃₋₁₀ cycloalkyl and C₃₋₆ cycloalkyl optionally substituted by hydroxyl, cyano, halogen, or C₁₋₆ alkyl; in 3- to 10-membered heterocycloalkyl, the heteroatom is independently selected from one or more of N, O and S, and the number of the heteroatom is independently 1-3; preferably, R⁵ is hydrogen, C₁₋₆ alkyl, 3- to 10-membered heterocycloalkyl, C₃₋₁₀ cycloalkyl, C₆₋₁₀ aryl, C₅₋₁₀ heteroaryl, or

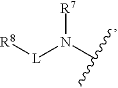

wherein R⁷ is hydrogen, or C₁₋₆ alkyl; R⁸ is C₁₋₆alkyl, 3- to 10-membered heterocycloalkyl; the 3- to 10-membered heterocycloalkyl optionally substituted by C₁₋₆ alkyl, C₆₋₁₀ aryl, —CO—O—C₃₋₆ cycloalkyl or —CO—O—C₁₋₆ alkyl; the C₃₋₁₀ cycloalkyl and C₃₋₆ cycloalkyl optionally substituted by hydroxyl, cyano, halogen, or C₁₋₆ alkyl;
in 3- to 10-membered heterocycloalkyl, the heteroatom is independently selected from one or more of N, O and S, and the number of the heteroatom is independently 1-3; more preferably, R⁵ is hydrogen, C₁₋₆ alkyl, or

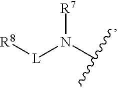

wherein R⁷ is hydrogen, or C₁₋₆ alkyl.

In some embodiments of the present disclosure, R⁸ is C₁₋₆alkyl, 3- to 6-membered heterocycloalkyl; the 3- to 6-membered heterocycloalkyl optionally substituted by C₆₋₁₀ aryl or —CO—O—C₁₋₆alkyl; in 3- to 6-membered heterocycloalkyl, the heteroatom is independently selected from one or two of N and 0, and the number of the heteroatom is independently 1-2.

In some embodiments of the present disclosure, A is

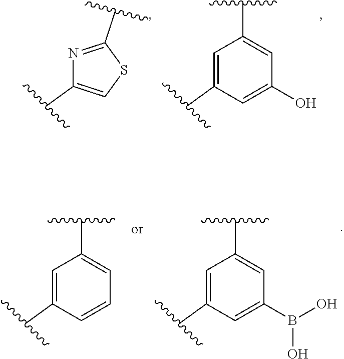 [SMILES: *c1csc(*)n1 ; *c1cc(*)cc(O)c1 ; *c1cccc(*)c1 ; *c1cc(*)cc(B(O)O)c1]

In some embodiments of the present disclosure, G is

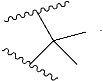 [SMILES: *C(*)(C)C]

In some embodiments of the present disclosure,

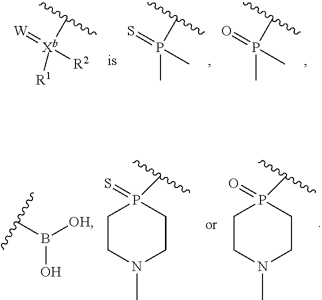

In some embodiments of the present disclosure, R³ is

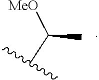 [SMILES: *[C@H](C)OC]

In some embodiments of the present disclosure, R⁴ is

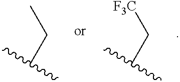

In some embodiments of the present disclosure,

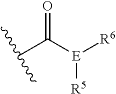

is

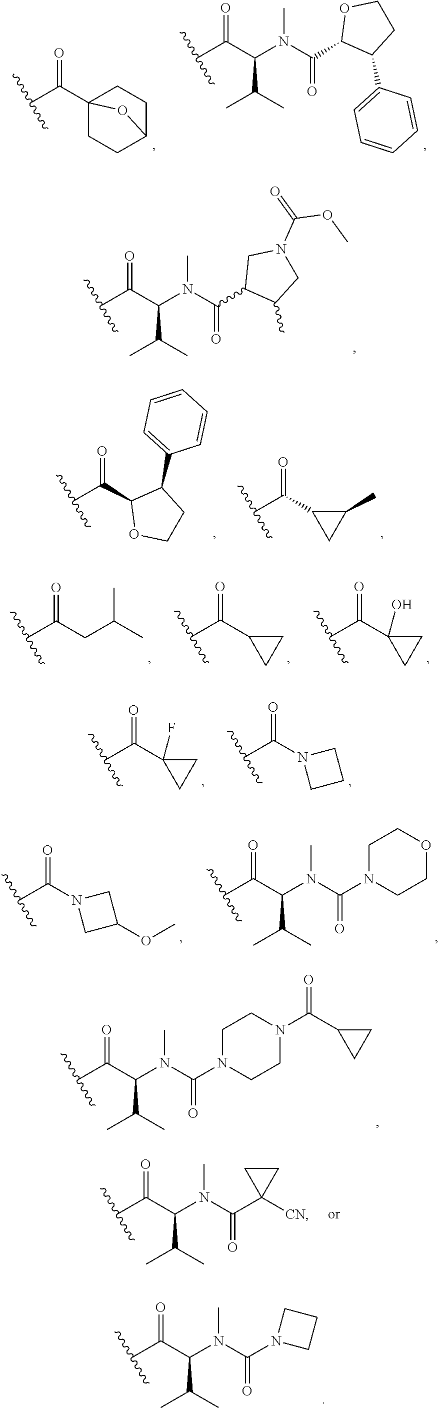

In some embodiments of the present disclosure, the “halogen” is F, Cl, Br or I; preferably, F.

The present invention provides a compound 1, a pharmaceutically acceptable salt thereof, a solvate thereof or a solvate of the pharmaceutically acceptable salt,
wherein, the compound 1 is selected from any one of the following compounds:

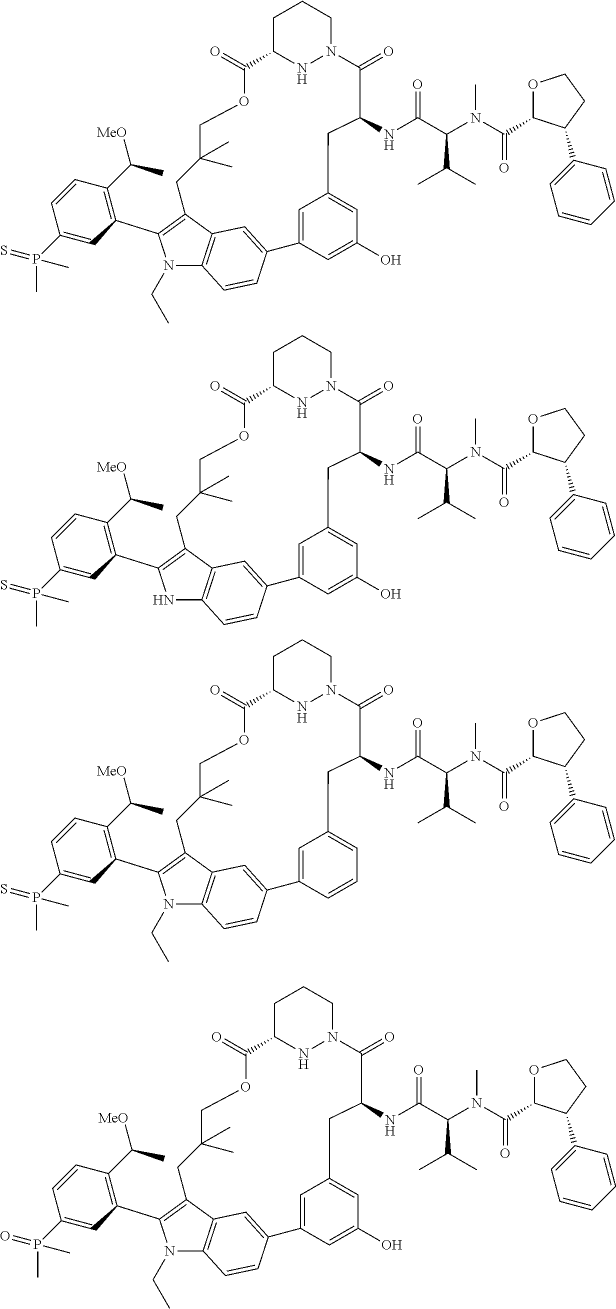

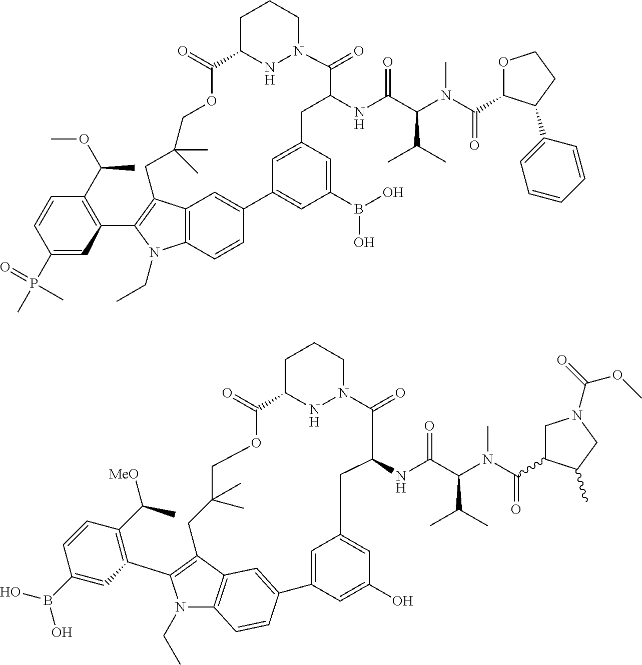

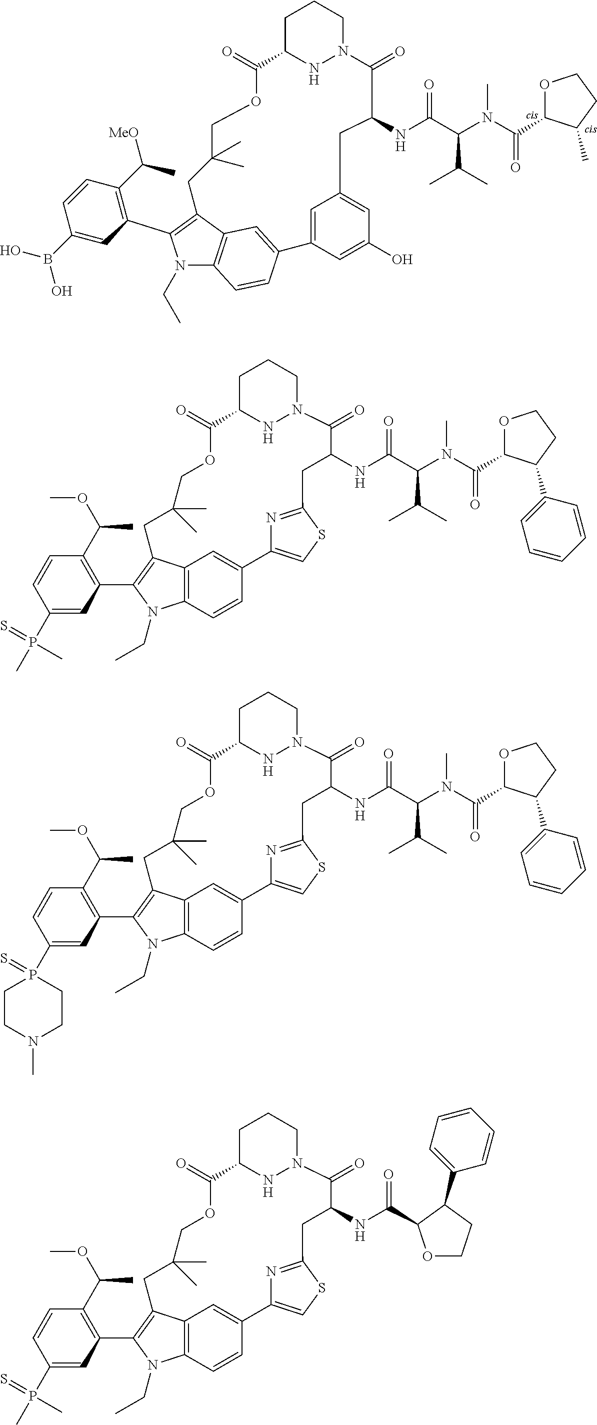

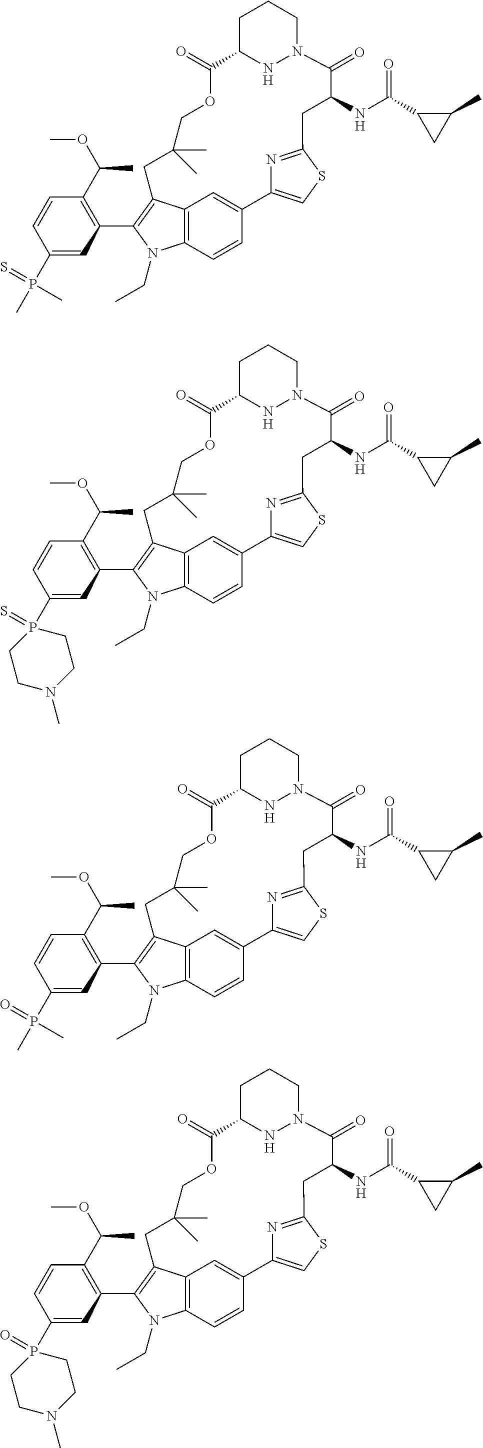

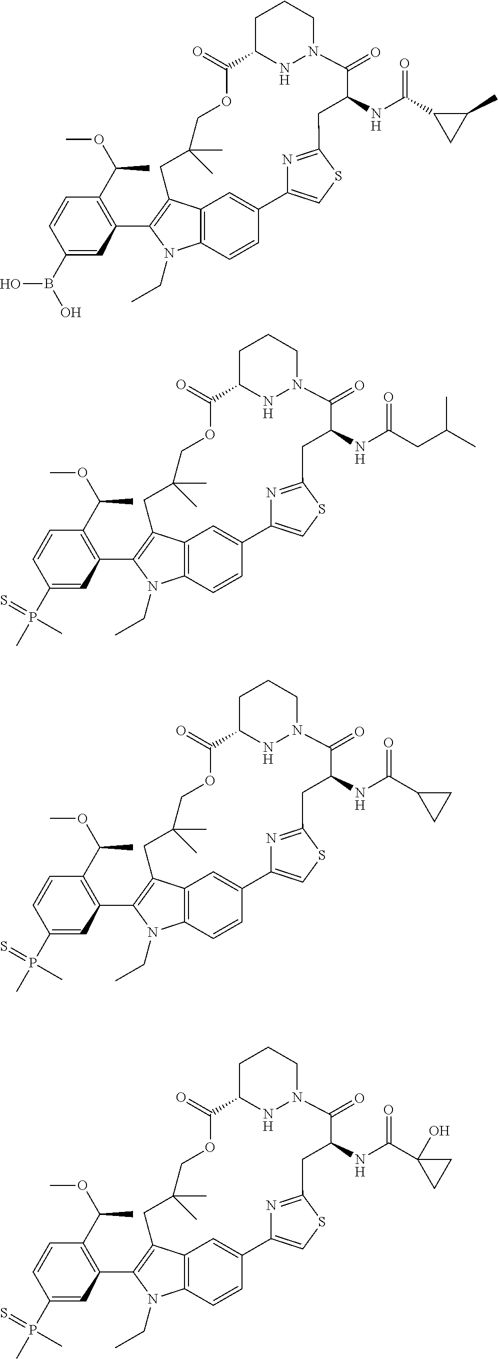

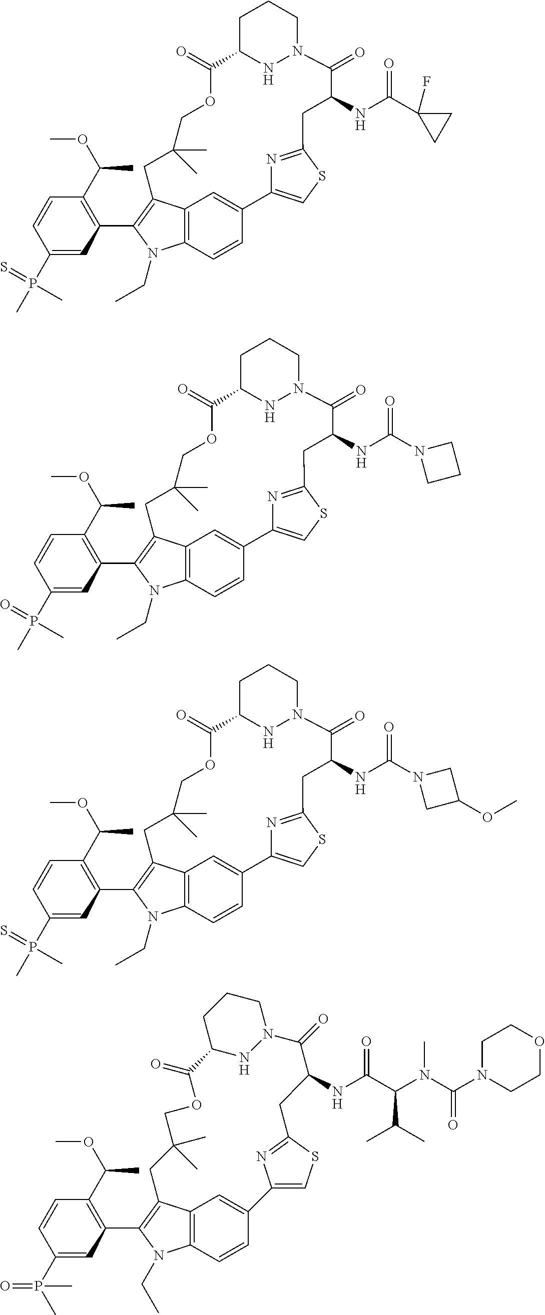

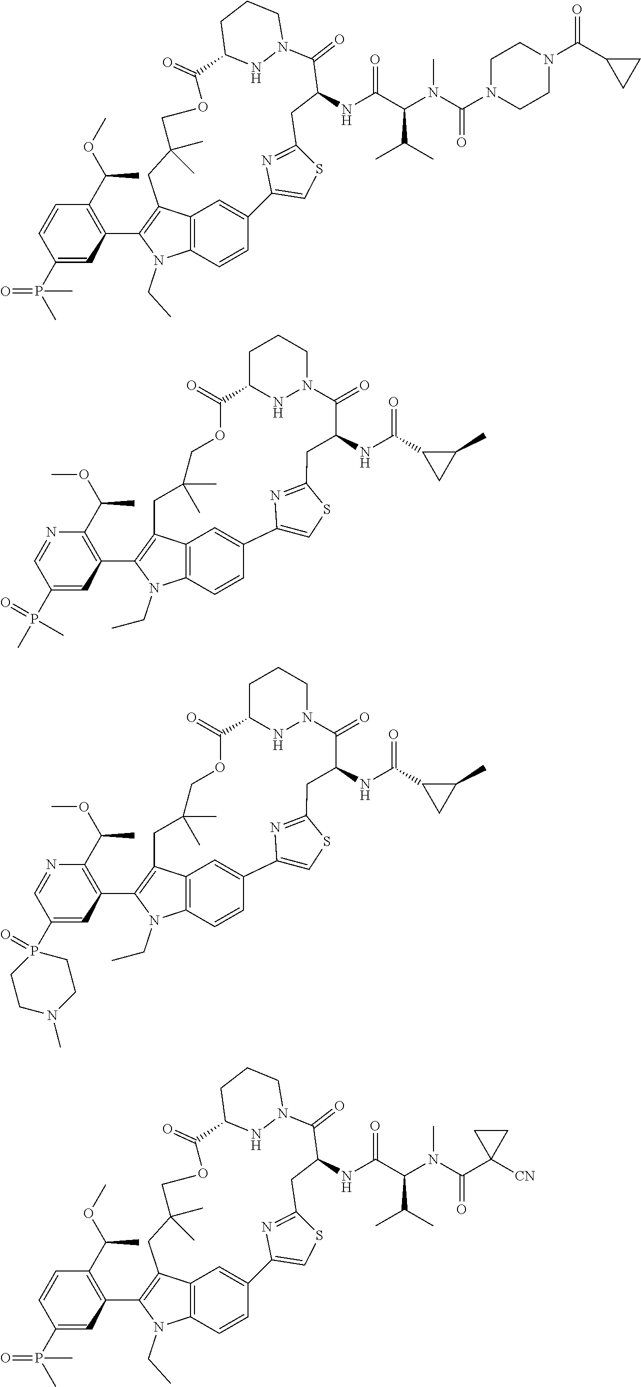

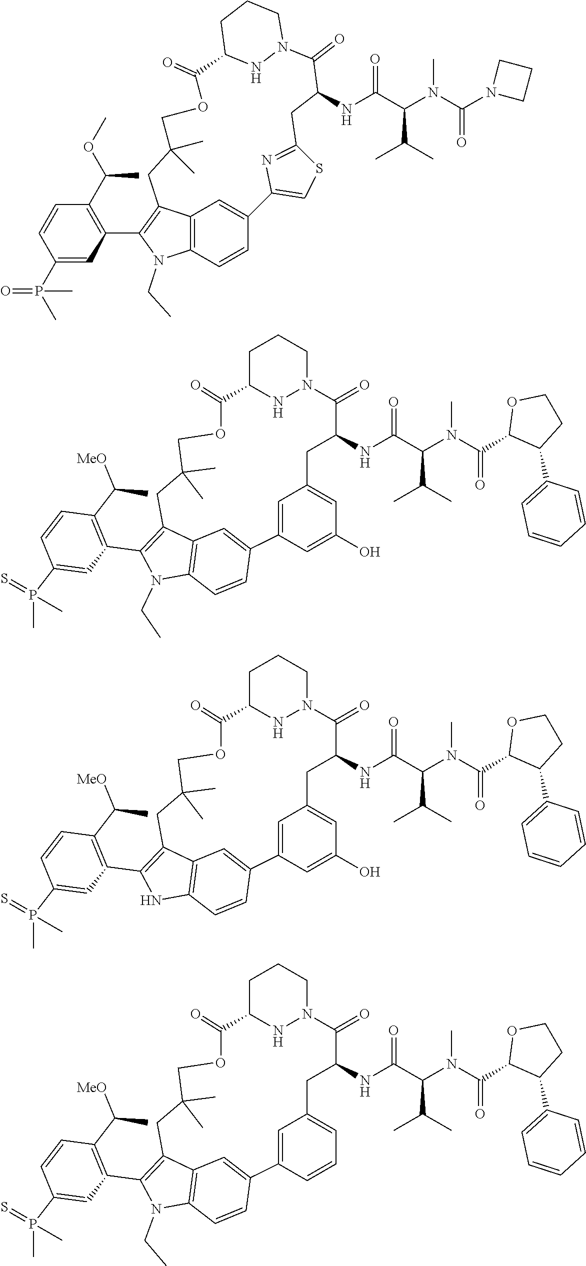

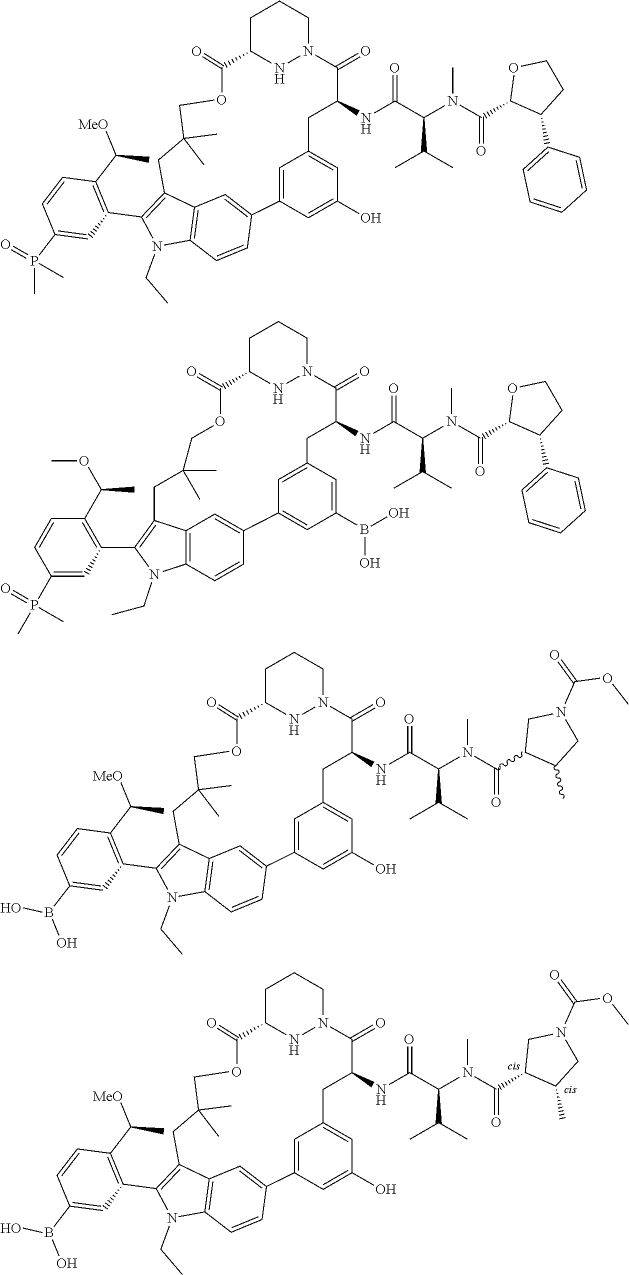

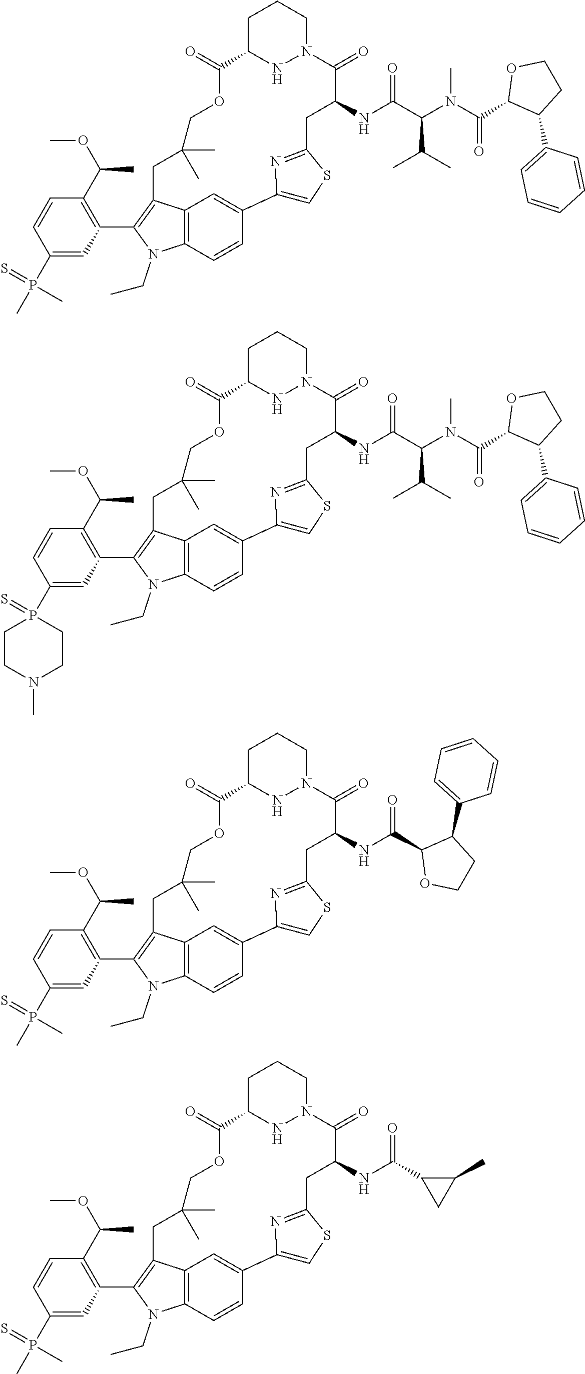

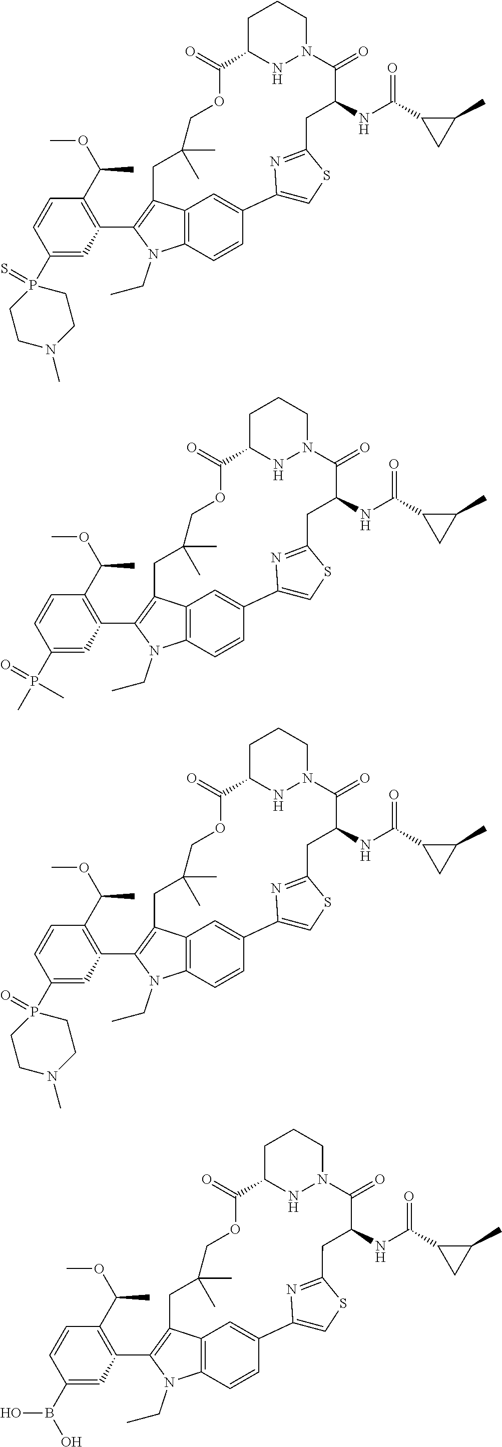

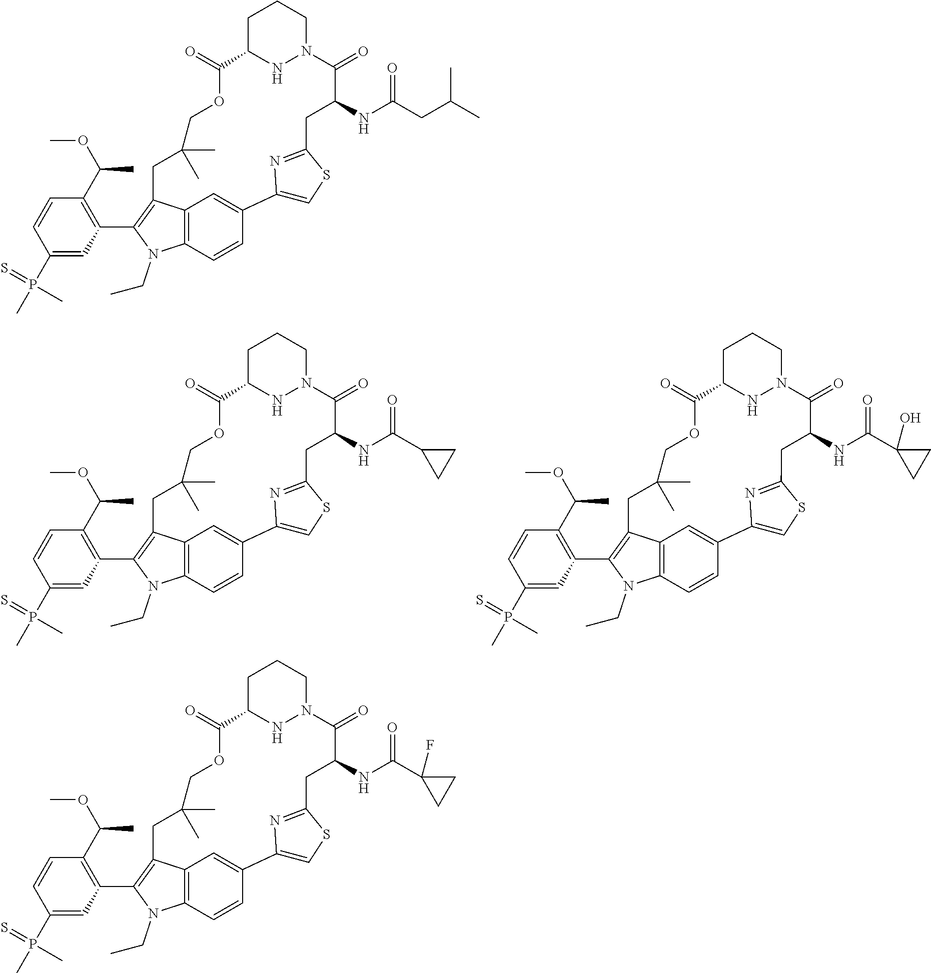

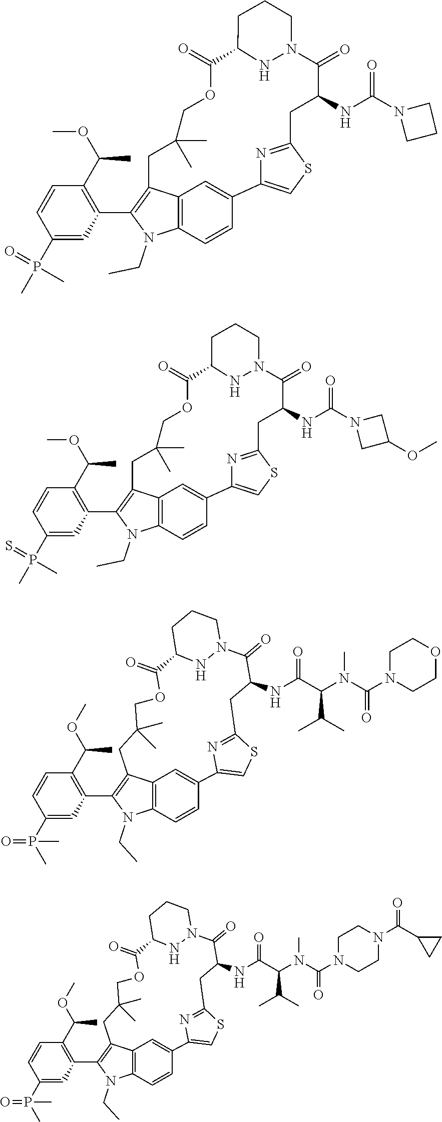

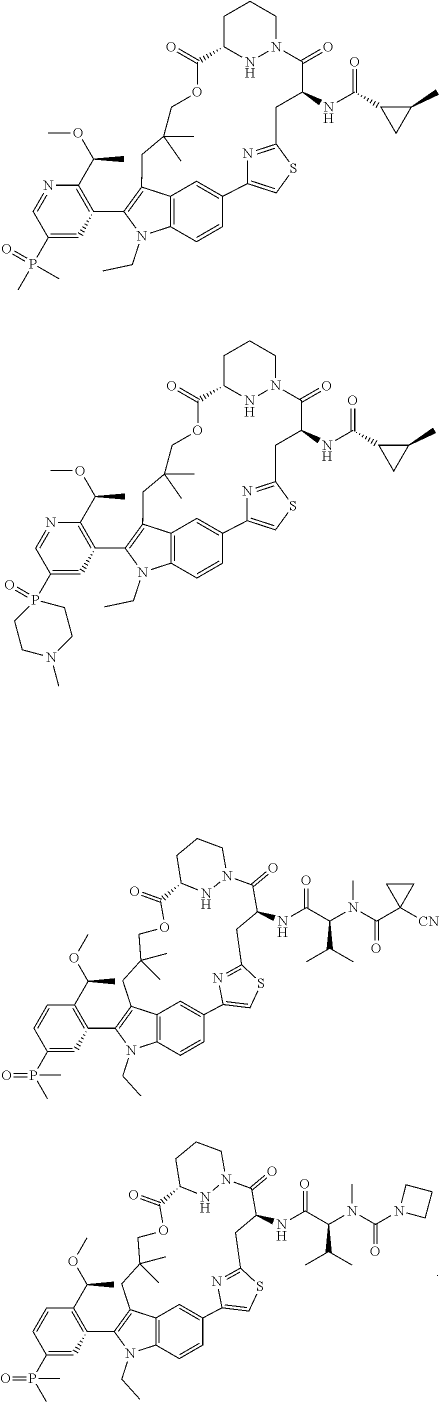

The present invention also provides a pharmaceutical composition comprising a compound, wherein a pharmaceutically acceptable salt thereof, a solvate thereof or a solvate of the pharmaceutically acceptable salt, and a pharmaceutically acceptable carrier, the compound is the compound 1 as defined above.

The present invention also provides a use of a compound, wherein a pharmaceutically acceptable salt thereof, a solvate thereof or a solvate of the pharmaceutically acceptable salt, or the pharmaceutical composition as defined above in the preparation of a medicament used for the treatment of cancer, wherein the compound is the compound 1 as defined above.

In some embodiments of the present disclosure, wherein the cancer is a Ras-driven cancer.

In some embodiments of the present disclosure, wherein the cancer comprises a Ras mutation.

In some embodiments of the present disclosure, wherein the Ras mutation is a position 12, 13 or 61.

In some embodiments of the present disclosure, wherein the Ras mutation is at position 12.

In some embodiments of the present disclosure, wherein the Ras mutation is selected from the group consisting of G12C, G12D, G12V, G12A, G12R, G12S, G13C, G13D, Q61H, Q61R and Q61L, or a combination thereof.

In some embodiments of the present disclosure, wherein the Ras mutation is a position selected from the group consisting of G12D, G12V, and G12R, or a combination thereof.

In some embodiments of the present disclosure, wherein the cancer is pancreatic cancer, appendiceal cancer, small bowel cancer, colorectal cancer, ampullary cancer, non-small cell lung cancer, cervical cancer, lung cancer, endometrial cancer, acute myeloid leukemia, gastrointestinal neuroendocrine tumour, uterine endometrioid carcinoma, oesophagogastric cancer, bladder cancer, ovarian cancer, melanoma, multiple myeloma, thyroid gland adenocarcinoma, a myelodysplastic syndrome, or squamous cell lung carcinoma.

In some embodiments of the present disclosure, wherein the cancer is pancreatic cancer, lung cancer, or colorectal cancer.

In some embodiments of the present disclosure, wherein the Ras protein is KRAS.

The present invention also provided is a method of treating cancer in a subject in need thereof, the method comprising administering to the subject a therapeutically effective amount of a pharmaceutically acceptable salt thereof, a solvate thereof or a solvate of the pharmaceutically acceptable salt or the pharmaceutical composition as defined above, wherein the compound is the compound 1 as defined above.

In some embodiments of the present disclosure, wherein the cancer is a Ras-driven cancer.

In some embodiments of the present disclosure, wherein the cancer comprises a Ras mutation.

In some embodiments of the present disclosure, wherein the Ras mutation is a position 12, 13 or 61.

In some embodiments of the present disclosure, wherein the Ras mutation is at position 12.

In some embodiments of the present disclosure, wherein the Ras mutation is at a position selected from the group consisting of G12C, G12D, G12V, G12A, G12R, G12S, G13C, G13D, Q61H, Q61R and Q61L, or a combination thereof.

In some embodiments of the present disclosure, wherein the Ras mutation is a position selected from the group consisting of G12D, G12V, and G12R, or a combination thereof.

In some embodiments of the present disclosure, wherein the cancer is pancreatic cancer, appendiceal cancer, small bowel cancer, colorectal cancer, ampullary cancer, non-small cell lung cancer, cervical cancer, lung cancer, endometrial cancer, acute myeloid leukemia, gastrointestinal neuroendocrine tumour, uterine endometrioid carcinoma, oesophagogastric cancer, bladder cancer, ovarian cancer, melanoma, multiple myeloma, thyroid gland adenocarcinoma, a myelodysplastic syndrome, or squamous cell lung carcinoma.

In some embodiments of the present disclosure, wherein the cancer is pancreatic cancer, lung cancer, or colorectal cancer.

In some embodiments of the present disclosure, wherein the Ras protein is KRAS.

In some embodiments, the method is provided of treating a Ras protein-related disorder in a subject in need thereof.

A compound, wherein the compound is selected from any one of the following compounds:

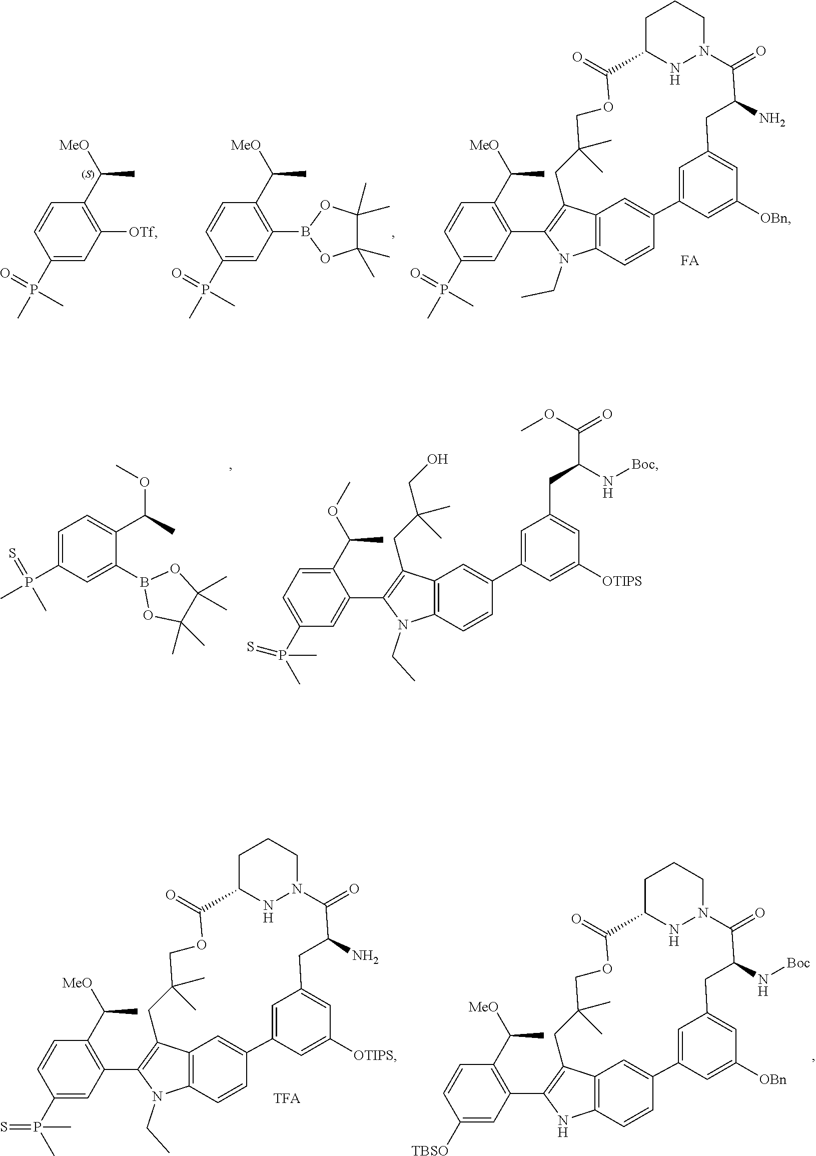

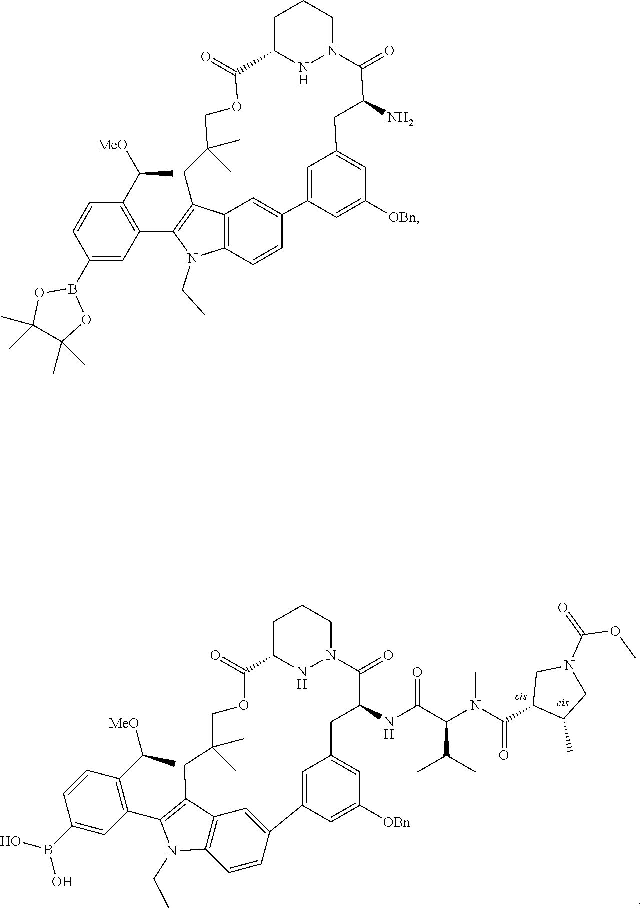 [SMILES: CCn1c(-c2cc(B3OC(C)(C)C(C)(C)O3)ccc2[C@H](C)OC)c2c3cc(ccc31)-c1cc(cc(OCc3ccccc3)c1)C[C@H](N)C(=O)N1CCC[C@H](N1)C(=O)OCC(C)(C)C2 ; CCn1c(-c2cc(B(O)O)ccc2[C@H](C)OC)c2c3cc(ccc31)-c1cc(cc(OCc3ccccc3)c1)C[C@H](NC(=O)[C@H](C(C)C)N(C)C(=O)[C@@H]1CN(C(=O)OC)C[C@@H]1C)C(=O)N1CCC[C@H](N1)C(=O)OCC(C)(C)C2]

### Term Definition

As used herein, the term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. When the compound of the present invention is basic, its corresponding salt can be conveniently prepared from pharmaceutically acceptable non-toxic acids, including inorganic and organic acids.

As used herein, the term “solvate” refers to solvent addition forms that contain either stoichiometric or non-stoichiometric amounts of solvent.
excipient that is useful in preparing a pharmaceutical composition that is generally safe, nontoxic and neither biologically nor otherwise undesirable, and includes carrier that is acceptable for veterinary use as well as human pharmaceutical use.

A “pharmaceutically acceptable carrier” as used herein includes both one and more than one such carrier. The term “pharmaceutically acceptable carrier” also encompasses “pharmaceutically acceptable excipient” and “pharmaceutically acceptable diluent”. The particular carrier used in the pharmaceutical compositions of the present disclosure will depend upon the means and purpose for which the compounds of the present disclosure are being applied.

As used herein, the term “alkyl” refers to a saturated linear or branched monovalent hydrocarbon group with a certain number of carbon atoms. C₁₋₆ alkyl refers to an alkyl group with 1-6 carbon atoms (for example, 1, 2, 3, 4, 5, 6). Examples of “alkyl” as used herein include, but are not limited to, methyl, ethyl, n-propyl, n-butyl, n-pentyl, n-hexyl, isobutyl, isopropyl, t-butyl and 1,1-dimethylpropyl.

The term “alkylene” is a divalent group which is connected to the rest of the molecule through two single bonds, and the rest definitions are the same as the term “alkyl”.

As used herein, the term “halogen” or “halo” refers to a fluorine (fluoro, F), chlorine (chloro, Cl), bromine (bromo, Br) or iodine (iodo, I) atom.

As used herein, the term “heteroaryl” refers to an aryl group having, in addition to carbon atoms, one, two, three or more heteroatoms independently selected from N, O and S. The heteroaryl group can be monocyclic or polycyclic in which at least one ring is aromatic.

The term “heteroarylene” is a divalent group which is connected to the rest of the molecule through two single bonds, and the rest definitions are the same as the term “heteroaryl”.

As used herein, the term “cycloalkyl” refers to a monovalent saturated cyclic hydrocarbon group, containing 3 to 10 ring carbon atoms having a single cyclic ring or multiple fused, spiro or bridged rings.

Examples of cycloalkyl include cyclopropyl, cyclobutyl, cyclopentyl, cyclohexyl, cycloheptyl, cyclooctyl, cyclononalkyl, cyclodecyl.

The term “cycloalkylene” is a divalent group which is connected to the rest of the molecule through two single bonds, and the rest definitions are the same as the term “cycloalkyl”.

As used herein, the term “heterocyclyl” refers to a saturated carbocyclic ring in which one or more of the carbon atoms have been replaced with heteroatoms, e.g. nitrogen, sulfur, and/or oxygen atoms. Examples of such heterocyclyl groups are

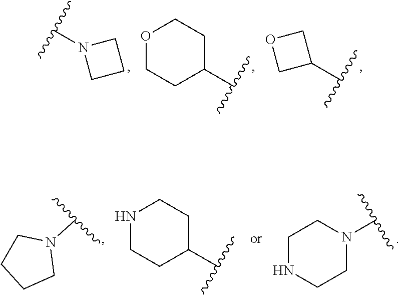

When the heterocyclyl has substituents, the substituents may be attached to any atom in the ring, provided that a stable chemical structure results.

The term “heterocycloalkylene,” is a divalent group which is connected to the rest of the molecule by two single bonds, and the rest definitions are the same as the term “heterocycloalkyl”.

As used herein, the term “alkoxy” refers to a straight or branched alkoxy group containing the specified number of carbon atoms. For example, C₁₋₆ alkoxy means a straight or branched alkoxy group containing at least 1, and at most 6, carbon atoms. Examples of “alkoxy” as used herein include, but not limited to, methoxy, ethoxy, prop-i-oxy, pro-2-oxy, pentoxy, hexyloxy, and the like.

As used herein, the term “alkylamino” refers to an alkyl moiety substituted by one or more amino moieties.

As used herein, the term “aryl” refers to monocyclic and polycyclic ring systems having a total of 6 to 10 ring members, wherein at least one ring in the system is aromatic. Examples of “aryl” include, but are not limited to, phenyl or naphthyl.

The term “arylene” is a divalent group which is connected to the rest of the molecule through two single bonds, and the rest definitions are the same as the term “aryl”.

As used herein, the term “C₁₋₆haloalkoxyl” refers to an alkoxyl group in which one or more hydrogen atoms are replaced by a halogen, for example, —OCF₃.

As used herein, the term “haloalkyl” refers to an alkyl group in which one or more hydrogen atoms are replaced by a halogen, for example, —CF₃.

As used herein, the term “C₁₋₆ alkoxyC₁₋₆alkyl” refers to a C₁₋₆alkyl group in which one or more hydrogen atoms are replaced by C₁₋₆ alkoxy or a C₁₋₆ alkoxy group in which one or more hydrogen atoms are replaced by C₁₋₆ alkyl.

The term “halogen” refers to fluorine, chlorine, bromine or iodine. The term “oxo” means that ═O, oxygen atoms replace two hydrogens on the same carbon atom, that is, carbonyl groups replace methylene groups.

As used herein, the term “optionally substituted” means an atom can be substituted with a substituent or not, unless otherwise specified.

It is to be understood that the foregoing description of two preferred embodiments is intended to be purely illustrative of the principles of the invention, rather than exhaustive thereof, and that changes and variations will be apparent to those skilled in the art, and that the present invention is not intended to be limited other than expressly set forth in the following claims.

### BRIEF DESCRIPTION OF THE DRAWINGS

FIG. 1 Example 2 was potently inhibited the interaction of RAF with active KRAS mutant protein or HRAS WT protein.

FIG. 2 Example 2 was potently inhibited the cell viability of RAS dependent cells.

### DETAILED DESCRIPTION OF THE PREFERRED EMBODIMENT

### Methods of Synthesis

The compounds of the present invention can be prepared by any conventional means. Suitable processes for synthesizing these compounds as well as their starting materials are provided in the schemes below and in the examples. All substituents are as defined above unless otherwise indicated. Furthermore, and unless explicitly otherwise stated, all reactions, reaction conditions, abbreviations and symbols have the meanings well known to a person of ordinary skill in organic chemistry.

General synthetic procedure for preparing compounds as formula (I) are shown below:

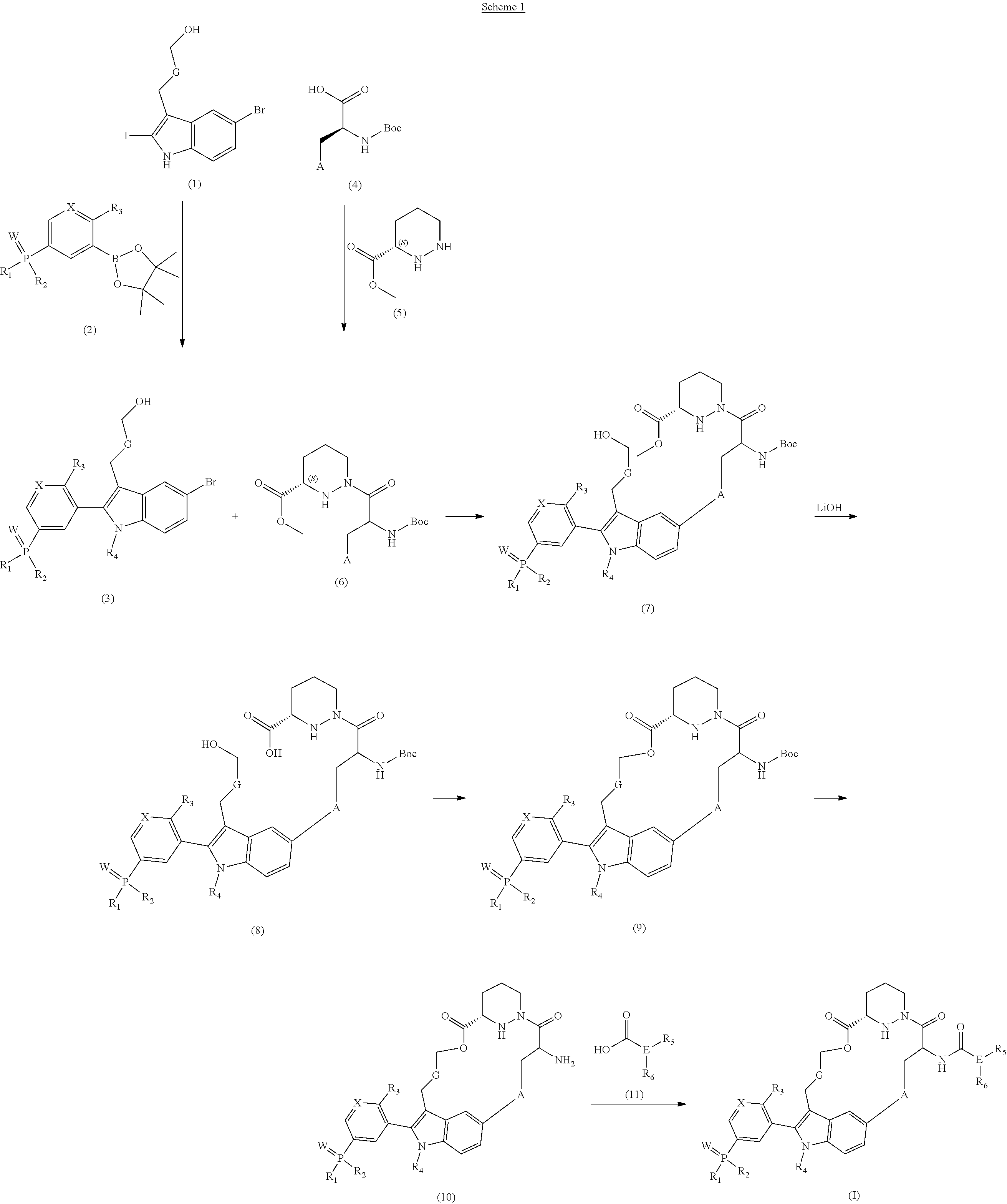

Scheme 1 illustrates the preparation of compound of formula (1). At first the coupling of compounds of formula (1) and (2) can be obtained through standard Suzuki coupling conditions (for example Pd(dppf)Cl₂ and K₂CO₃) to provide compound of formula (3). Compound of formula (6) was attained by a routine coupling condition between acid (4) and amine (5) with coupling reagents, such as HATU, EDCI/HOBt or PyBOP etc, in the presence of an organic base, such as Et₃N, DIPEA or pyridine. Compound of formula (3) was coupled with compound of formula (6) under Suzuki coupling conditions (for example Pd(dppf)Cl₂ and K₂CO₃) to give compound of formula (7), which was hydrolyzed to provide acid of formula (8) under a base, such as LiOH. The intramolecular coupling of compound of formula (8) with coupling reagents, such as EDCI/HOBt and HATU, to give compound of formula (9). Deprotection of compound (9) can afford compound of formula (10) in the presence of an acid, such as TFA. Compound of formula (1) can be obtained by a coupling reaction between acid (11) and amine (10) with coupling reagents, such as HATU, EDCI/HOBy or PyBOP, in the presence of a base, such as TEA, DIPEA or pyridine.

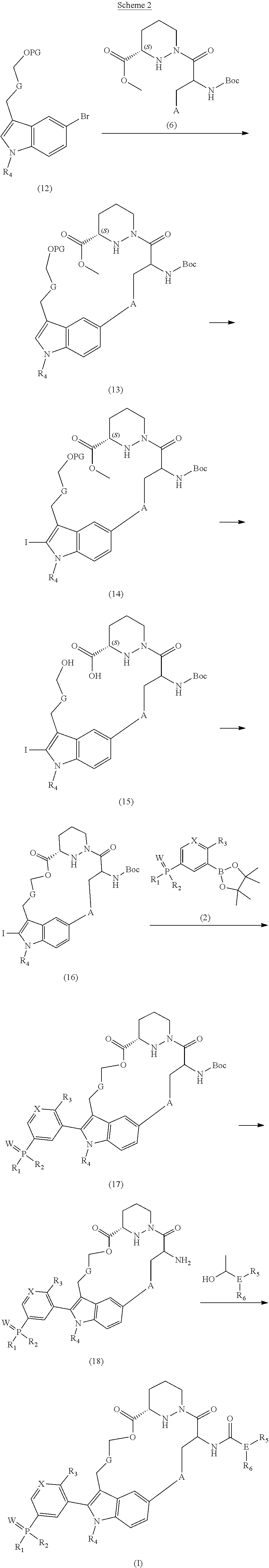

Scheme 2 describes an alternative method for preparation of compound of formula (I). Compound of formula (12) was coupled with compound of formula (6) under Suzuki coupling conditions (for example Pd(dppf)Cl₂ and K₂CO₃) to give compound of formula (13), which was converted to compound of formula (14) in the presence of iodization reagents, such I₂ or NIS. Deprotection of compound of formula (14) to provide compound of formula (15), such as hydrolysis of the ester with LiOH, or with TBAF deprotecting silicon protective group on hydroxyl group. The intramolecular coupling of compound of formula (15) with coupling reagents, such as EDCI/HOBt and HATU, to afford compound of formula (16), which as further coupled with compound of formula (2) under Suzuki coupling conditions ((for example Pd(dppf)Cl₂ and K₂CO₃) to give compound of formula (17). Compound of formula (17) was reacted in the similar method as described in the scheme 1 to give compound of formula (I).

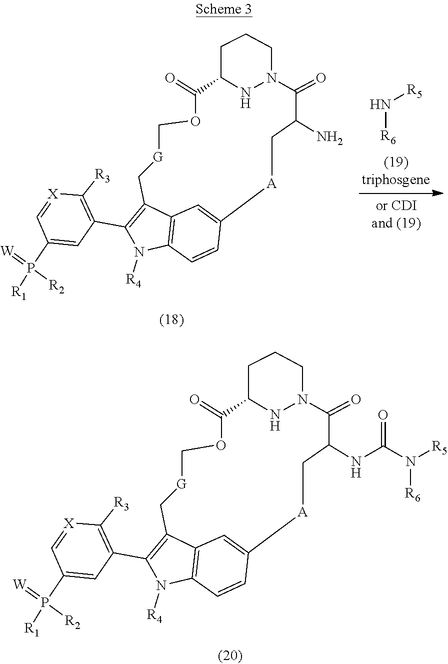

Compound of formula (20) as illustrated in scheme 3 can be obtained by treatment compound (18) and (19) with triphosgene or CDI in the presence of a base, such as TEA, DIPEA or pyridine.

### Preparation of Intermediate

### Intermediate A

### tert-butyl ((6³S,4S)-2⁵-(benzyloxy)-1²-iodo-10,10-dimethyl-5,7-dioxo-6¹,6²,6³,6⁴,6⁵,6⁶-hexahydro-1′H-8-oxa-1(5,3)-indola-6(1,3)-pyridazina-2(1,3)-benzenacycloundecaphane-4-yl)carbamate

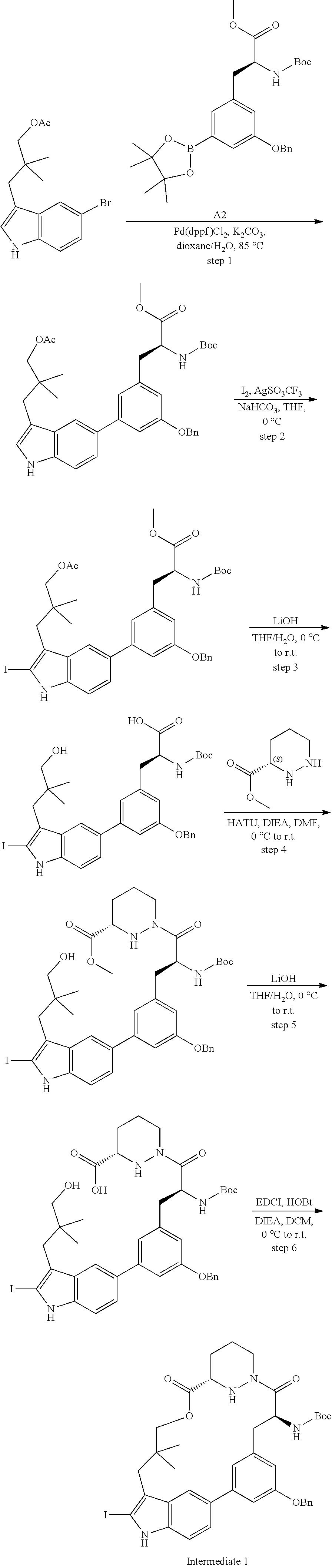

### Step 1. Synthesis of methyl (S)-3-(3-(3-(3-acetoxy-2,2-dimethylpropyl)-1H-indol-5-yl)-5-(benzyloxy)phenyl)-2-((tert-butoxycarbonyl)amino)propanoate

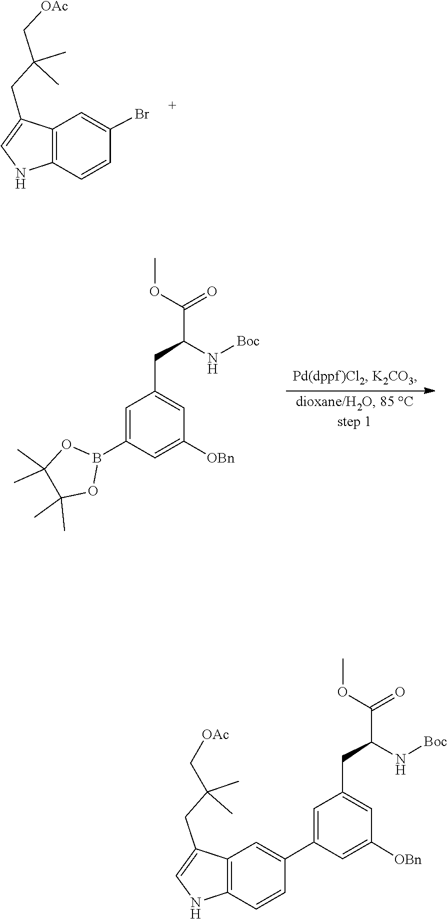 [SMILES: CC(=O)OCC(C)(C)Cc1c[nH]c2ccc(Br)cc12 ; COC(=O)[C@H](Cc1cc(OCc2ccccc2)cc(B2OC(C)(C)C(C)(C)O2)c1)NC(=O)OC(C)(C)C ; COC(=O)[C@H](Cc1cc(OCc2ccccc2)cc(-c2ccc3[nH]cc(CC(C)(C)COC(C)=O)c3c2)c1)NC(=O)OC(C)(C)C]

To a stirred solution of 3-(5-bromo-1H-indol-3-yl)-2,2-dimethylpropyl acetate (10.3 g, 31.8 mmol) and methyl (S)-3-(3-(benzyloxy)-5-(4,4,5,5-tetramethyl-1,3,2-dioxaborolan-2-yl)phenyl)-2-((tert-butoxycarbonyl)amino)propanoate (19.5 g, 38.1 mmol) in pre-mixtured solvents dioxane/H₂O (240 mL, 4:1, V:V) was added Pd(dppt)Cl₂ (2.3 g, 3.2 mmol) and K₂CO₃ (11.0 g, 79.5 mmol). Then the reaction mixture was stirred at 80° C. for 4 h under N₂ atmosphere and the reaction was monitored by LCMS. After completion, the reaction solution was concentrated under reduced pressure to remove dioxane. Then the reaction mixture was diluted with H₂O (20 mL) and extracted with EtOAc (50 mL×4). The combined organic phase was washed with saturated NaCl (20 mL), then dried over with Na₂SO₄, following with concentration under reduced pressure to obtain crude one, which was purified by silica gel column eluting with EtOAc/petroleum ether from 0% to 50% to afford methyl (S)-3-(3-(3-(3-acetoxy-2,2-dimethylpropyl)-1H-indol-5-yl)-5-(benzyloxy)phenyl)-2-((tert-butoxycarbonyl)amino)propanoate (14.0 g, 70%) as a light yellow solid.

LCMS (ESI) calcd. for C₃₇H₄₄N₂O₇ [M−H]⁻ m/z 627.31, found: 627.7

### Step 2. Synthesis of methyl (S)-3-(3-(3-(3-acetoxy-2,2-dimethylpropyl)-2-iodo-1H-indol-5-yl)-5-(benzyloxy)phenyl)-2-((tert-butoxycarbonyl)amino)propanoate

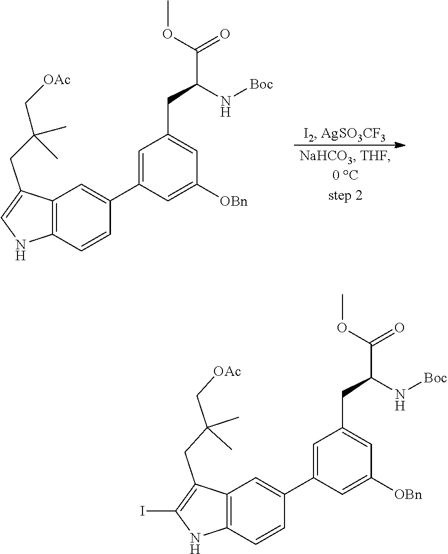 [SMILES: COC(=O)[C@H](Cc1cc(OCc2ccccc2)cc(-c2ccc3[nH]cc(CC(C)(C)COC(C)=O)c3c2)c1)NC(=O)OC(C)(C)C ; COC(=O)[C@H](Cc1cc(OCc2ccccc2)cc(-c2ccc3[nH]c(I)c(CC(C)(C)COC(C)=O)c3c2)c1)NC(=O)OC(C)(C)C]

To a stirred solution of methyl (S)-3-(3-(3-(3-acetoxy-2,2-dimethylpropyl)-1H-indol-5-yl)-5-(benzyloxy)phenyl)-2-((tert-butoxycarbonyl)amino)propanoate (14.13 g, 22.5 mmol) and NaHCO₃ (2.25 g, 27.0 mmol) in THF (180 mL) under ice-water bath was added AgSO₃CF₃ (6.94 g, 27.0 mmol) in THF (65 mL) and I₂ (5.71 g, 22.5 mmol) in THF (80 mL) by dropwise to the reaction mixture. The reaction was stirred at 0° C. for 2 h under N₂ atmosphere and the reaction was monitored by LCMS. After completion, the reaction quenched with saturated Na₂SO₃ (50 mL) and stirred for 30 mins, then filtered, the filtration was extracted with EtOAc (50 mL×4), the combined organic phase was washed with saturated NaCl (25 mL), then dried over with Na₂SO₄, following with concentration under reduced pressure to obtain crude one, which was purified by silica gel column eluting with EtOAc/PE gradient from 0% to 40% to afford methyl (S)-3-(3-(3-(3-acetoxy-2,2-dimethylpropyl)-2-iodo-1H-indol-5-yl)-5-(benzyloxy)phenyl)-2-((tert-butoxycarbonyl)amino)propanoate (14.0 g, 83%) as a light yellow solid.

LCMS (ESI) calcd. for C₃₇H₄₃IN₂O₇[M+H]⁺ m/z 755.21, found: 655.7

### Step 3. Synthesis of (S)-3-(3-(benzyloxy)-5-(3-(3-hydroxy-2,2-dimethylpropyl)-2-iodo-1H-indol-5-yl)phenyl)-2-((tert-butoxycarbonyl)amino)propanoic acid

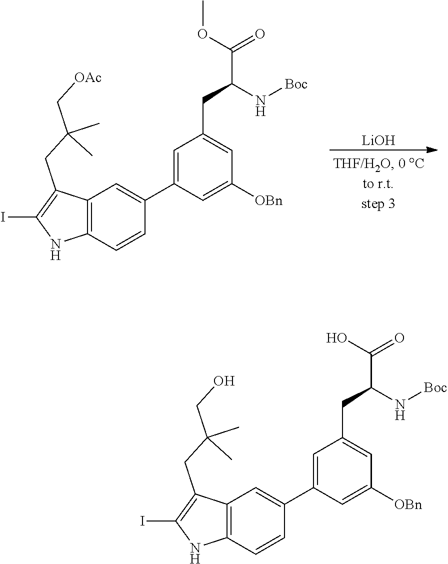 [SMILES: COC(=O)[C@H](Cc1cc(OCc2ccccc2)cc(-c2ccc3[nH]c(I)c(CC(C)(C)COC(C)=O)c3c2)c1)NC(=O)OC(C)(C)C ; "LiOH" ; CC(C)(CO)Cc1c(I)[nH]c2ccc(-c3cc(C[C@H](NC(=O)OC(C)(C)C)C(=O)O)cc(OCc4ccccc4)c3)cc12]

To a stirred solution of methyl (S)-3-(3-(3-(3-acetoxy-2,2-dimethylpropyl)-2-iodo-1H-indol-5-yl)-5-(benzyloxy)phenyl)-2-((tert-butoxycarbonyl)amino)propanoate (14.0 g, 18.6 mmol) in pre-mixtured solvents THF/MeOH/H₂O (180 mL, V:V:V=1:1:1) was added LiOH (3.04 g, 74.1 mmol) by one portion at 0° C. The reaction mixture was stirred at room temperature until the reaction was completed and the reaction was monitored by LCMS. After completion, the reaction solution was adjusted to pH=5-7 with 1M HCl and generated a large amount of precipitation. The resulting mixture was filtrated and the filter cake was washed with H₂O (20 mL×2). The mixture was dried under reduced pressure to obtain crude (S)-3-(3-(benzyloxy)-5-(3-(3-hydroxy-2,2-dimethylpropyl)-2-iodo-1H-indol-5-yl)phenyl)-2-((tert-butoxycarbonyl)amino)propanoic acid (9.56 g, 73%) as an light yellow solid. Which was directly used for the next step without further purification.

LCMS (ESI) calcd. for C₃₇H₄₃IN₂O₇[M−H]⁻ m/z 697.57, found: 697.4

### Step 4. Synthesis of methyl (S)-1-((S)-3-(3-(benzyloxy)-5-(3-(3-hydroxy-2,2-dimethylpropyl)-2-iodo-1H-indol-5-yl)phenyl)-2-((tert-butoxycarbonyl)amino)propanoyl)hexahydropyridazine-3-carboxylate

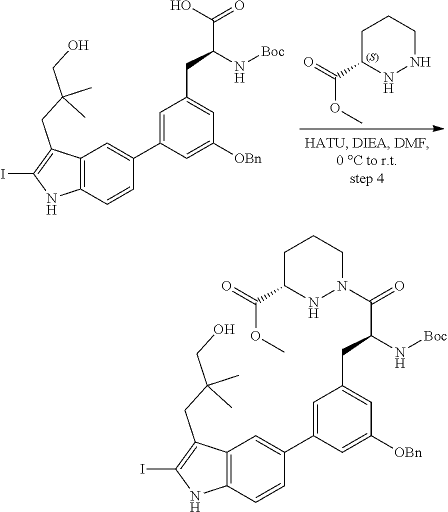

To a solution of crude (S)-3-(3-(benzyloxy)-5-(3-(3-hydroxy-2,2-dimethylpropyl)-2-iodo-1H-indol-5-yl)phenyl)-2-((tert-butoxycarbonyl)amino)propanoic acid (9.56 g, 13.7 mmol) in dry DMF (100 ml) was added DIEA (6.36 g, 49.3 mmol) and HATU (6.25 g, 16.4 mmol). The resulting mixture was stirred for 5 mins under ice-water bath, then methyl (S)-hexahydropyridazine-3-carboxylate bis(2,2,2-trifluoroacetate) (7.64 g, 20.54 mmol) in dry DMF (44 ml) was added by dropwise. The reaction temperature was gradually increased to room temperature and stirred for 2 h. The reaction was monitored by LCMS. After completion. The reaction was diluted with saturated NaCl (250 mL) and was extracted with EtOAc (100 mL×4). The combined organic phase was washed with saturated NaCl (50 mL), then died over with Na₂SO₄, following with concentration under reduced pressure to obtain crude one, which was purified by silica gel column eluting with MeOH/DCM from 0% to 5% to afford methyl (S)-1-((S)-3-(3-(benzyloxy)-5-(3-(3-hydroxy-2,2-dimethylpropyl)-2-iodo-1H-indol-5-yl) phenyl)-2-((tert-butoxycarbonyl)amino)propanoyl)hexahydropyridazine-3-carboxylate (9.0 g, 79%) as a light yellow solid.

LCMS (ESI) calcd. for C₄₀H₄₉IN₄O₇[M+H]⁺ m/z 825.27, found: 725.15

### Step 5. Synthesis of (S)-1-((S)-3-(3-(benzyloxy)-5-(3-(3-hydroxy-2,2-dimethylpropyl)-2-iodo-1H-indol-5-yl)phenyl)-2-((tert-butoxycarbonyl)amino)propanoyl)hexahydropyridazine-3-carboxylic acid

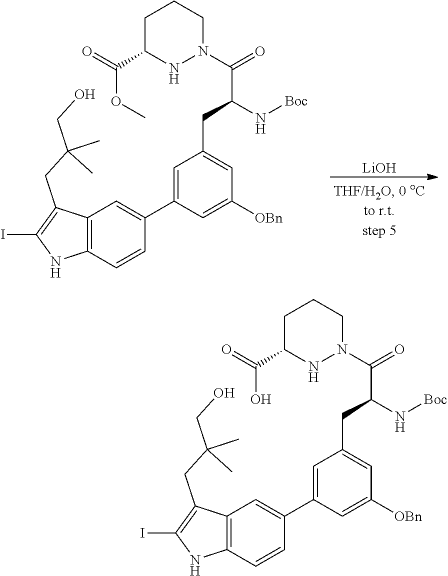 [SMILES: COC(=O)[C@@H]1CCCN(C(=O)[C@H](Cc2cc(OCc3ccccc3)cc(-c3ccc4[nH]c(I)c(CC(C)(C)CO)c4c3)c2)NC(=O)OC(C)(C)C)N1 ; "LiOH" ; CC(C)(CO)Cc1c(I)[nH]c2ccc(-c3cc(C[C@H](NC(=O)OC(C)(C)C)C(=O)N4CCC[C@@H](C(=O)O)N4)cc(OCc4ccccc4)c3)cc12]

To a solution of methyl (S)-1-((S)-3-(3-(benzyloxy)-5-(3-(3-hydroxy-2,2-dimethylpropyl)-2-iodo-1H-indol-5-yl)phenyl)-2-((tert-butoxycarbonyl)amino)propanoyl)hexahydropyridazine-3-carboxylate (8.5 g, 10.31 mmol) in solvents THF/MeOH/H₂O (225 ml, V:V:V=1:1:1) was added LiOH (1.06 g, 25.78 mmol) at room temperature. The reaction solution was stirred at room temperature for 2 h and the reaction was monitored by LCMS. After completion, the reaction solution was adjusted to pH=5-7 with 1M HCl under ice-water bath. Then the reaction solution was extracted with EtOAc (250 mL×4). The combined organic phase was washed with saturated NaCl (50 mL), then died over with Na₂SO₄, following with concentration under reduced pressure to obtain crude (S)-1-((S)-3-(3-(benzyloxy)-5-(3-(3-hydroxy-2,2-dimethylpropyl)-2-iodo-1H-indol-5-yl)phenyl)-2-((tert-butoxycarbonyl)amino)propanoyl)hexahydropyridazine-3-carboxylic acid (7.9 g, assumed 95%) as a white solid.

LCMS (ESI) calcd. for C₃₉H₄₇IN₄O₇[M+H]⁺ m/z 811.25, found: 711.16

### Step 6. Synthesis of tert-butyl ((6³S,4S)-2⁵-(benzyloxy)-1²-iodo-10,10-dimethyl-5,7-dioxo-6¹,6²,6³,6⁴,6⁵,6⁶-hexahydro-1H-8-oxa-1(5,3)-indola-6(1,3)-pyridazina-2(1,3)-benzenacycloundecaphane-4-yl)carbamate (intermediate 1)

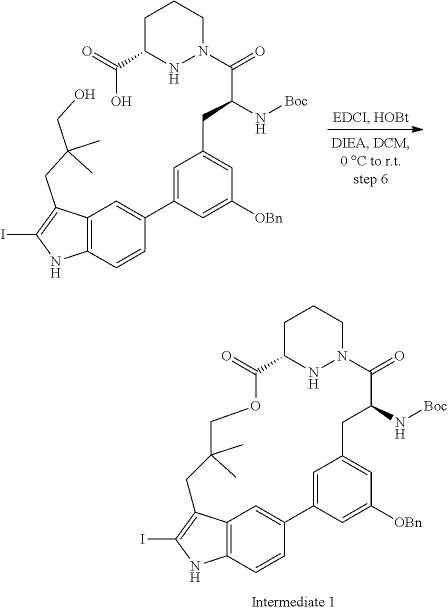 [SMILES: CC(C)(CO)Cc1c(I)[nH]c2ccc(-c3cc(C[C@H](NC(=O)OC(C)(C)C)C(=O)N4CCC[C@@H](C(=O)O)N4)cc(OCc4ccccc4)c3)cc12 ; CC1(C)COC(=O)[C@@H]2CCCN(N2)C(=O)[C@@H](NC(=O)OC(C)(C)C)Cc2cc(OCc3ccccc3)cc(c2)-c2ccc3[nH]c(I)c(c3c2)C1]

To a solution of crude (S)-1-((S)-3-(3-(benzyloxy)-5-(3-(3-hydroxy-2,2-dimethylpropyl)-2-iodo-1H-indol-5-yl)phenyl)-2-((tert-butoxycarbonyl)amino)propanoyl)hexahydropyridazine-3-carboxylic acid (5.9 g, 7.28 mmol) and DIEA (2.82 g, 21.83 mmol) in dry DCM (1190 mL) under ice-water bath was added EDCI (4.19 g, 21.83 mmol) and HOBt (1.97 g, 14.55 mmol) by one portion. The reaction was gradually improved temperature to room temperature and stirred for 4 h. The reaction was monitored by LCMS. After completion, the reaction was washed with H₂O (100 mL×3), then dried over with Na₂SO₄, following with concentration under reduced pressure to obtain crude one which was purified by silica gel column eluting with MeOH/DCM from 0% to 50% to afford tert-butyl ((6³S,4S)-2⁵-(benzyloxy)-1²-iodo-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (4.6 g, 91%, intermediate 1) as a white solid. Which was directly used for the next step without further purification.

LCMS (ESI) calcd. for C₃₉H₄₅IN₄O₆ [M+H]⁺ m/z 793.24, found: 693.5

### Example 1

### (3-((6³S,4S)-1¹-ethyl-2⁵-hydroxy-4-((S)-2-((3S,4R)-1-(methoxycarbonyl)-N,4-dimethylpyrrolidine-3-carboxamido)-3-methylbutanamido)-10,10-dimethyl-5,7-dioxo-6¹,6²,6³,6⁴,6⁵,6⁶-hexahydro-1 ¹H-8-oxa-1(5,3)-indola-6(1,3)-pyridazina-2(1,3)-benzenacycloundecaphane-1²-yl)-4-((S)-1-methoxyethyl)phenyl)boronic acid

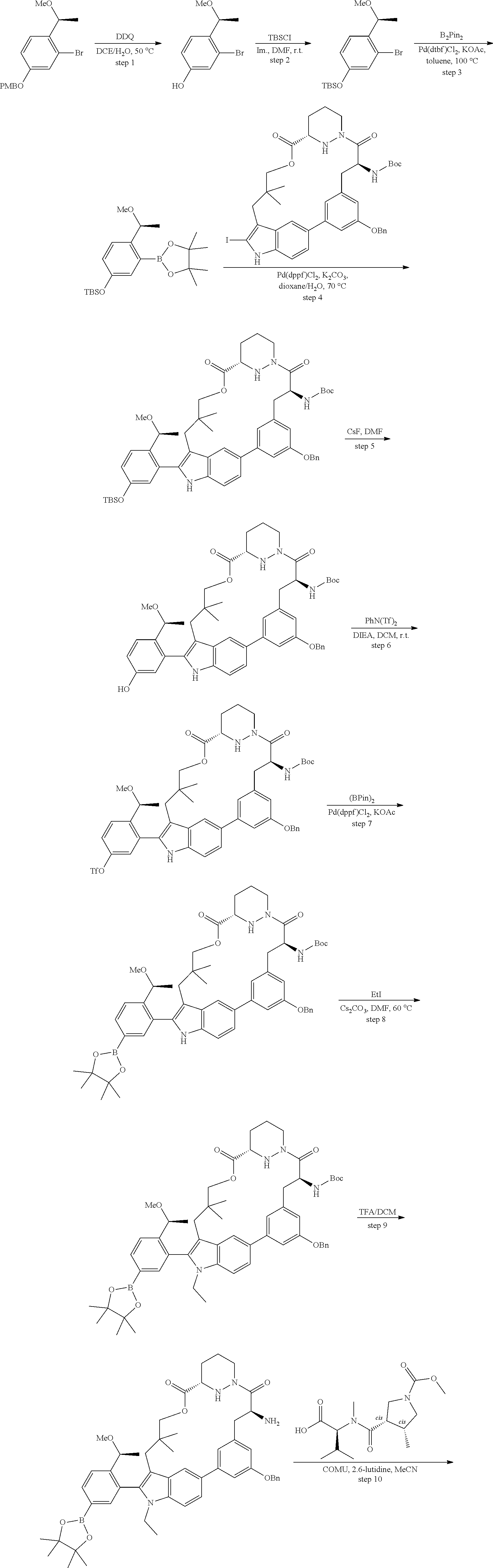

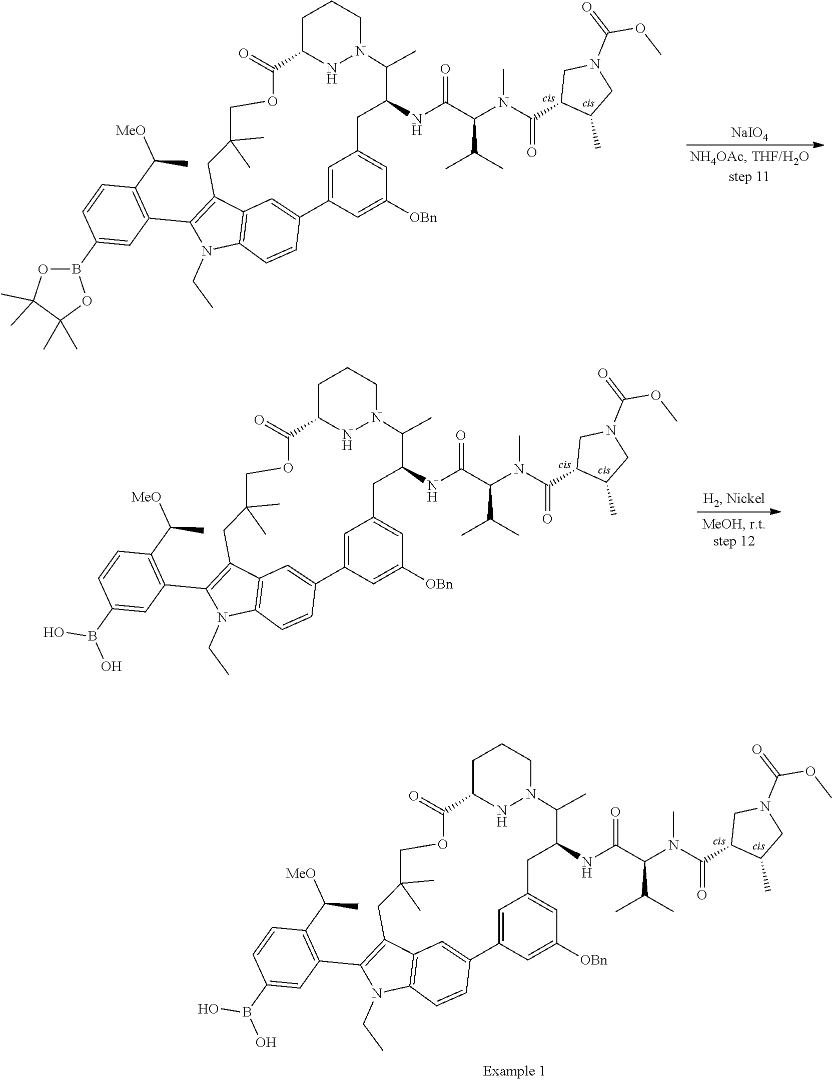

### Step 1. Synthesis of (S)-3-bromo-4-(1-methoxyethyl)phenol

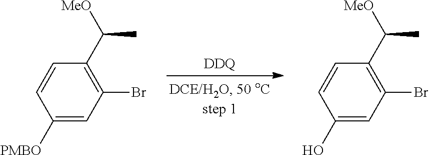

To a stirred solution of (S)-2-bromo-4-((4-methoxybenzyl)oxy)-1-(1-methoxyethyl)benzene (2.0 g, 5.7 mmol) in pre-mixtured solvents DCM/H₂O (54 mL, 17:1, V:V) was added DDQ (1.94 g, 8.6 mmol) to the reaction mixture at room temperature. The reaction was stirred at room temperature overnight and the reaction was monitored by LCMS. After completion, the reaction solution was filtered and the organic phase was washed with saturated NaCl (50 mL×2), then dried over with Na₂SO₄, following with concentration under reduced pressure to obtain crude one, which was purified by silica gel column eluting with EtOAc/petroleum ether from 0% to 50% to afford (S)-3-bromo-4-(1-methoxyethyl)phenol (350 mg, 26%) as a white solid.

### Step 2. Synthesis of (S)-(3-bromo-4-(1-methoxyethyl)phenoxy)(tert-butyl)dimethylsilane

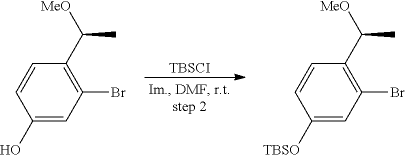

To a stirred solution of (S)-3-bromo-4-(1-methoxyethyl)phenol (400 mg, 0.173 mmol) in DMF (5 mL) was added imidzole (235.7 mg, 0.346 mmol) and TBSCl (313 mg, 0.208 mmol) to the reaction mixture at room temperature. The reaction was stirred at room temperature for 2 h under N₂ atmosphere, the reaction was monitored by LCMS. After completion, The reaction quenched with saturated NaCl (20 mL) and extracted with EtOAc (50 mL×4), the combined organic phase was washed with saturated NaCl (25 mL), then died over with Na₂SO₄, following with concentration under reduced pressure to obtain crude one, which was purified by silica gel column eluting with EtOAc/petroleum ether gradient from 0% to 3% to afford (S)-(3-bromo-4-(1-methoxyethyl)phenoxy)(tert-butyl)dimethylsilane (830 mg, assumed 100%) as a colorless oil.

### Step 3. Synthesis of (S)-tert-butyl(4-(1-methoxyethyl)-3-(4,4,5,5-tetramethyl-1,3,2-dioxaborolan-2-yl)phenoxy)dimethylsilane

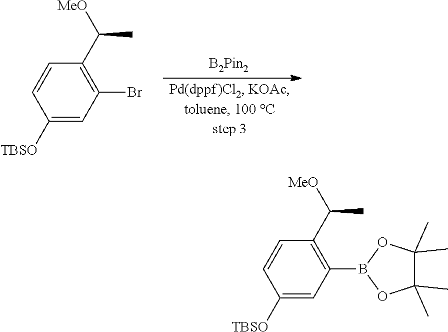 [SMILES: CO[C@@H](C)c1ccc(O[Si](C)(C)C(C)(C)C)cc1Br ; CO[C@@H](C)c1ccc(O[Si](C)(C)C(C)(C)C)cc1B1OC(C)(C)C(C)(C)O1]

To a solution of (S)-(3-bromo-4-(1-methoxyethyl)phenoxy)(tert-butyl)dimethylsilane (2.4 g, 6.96 mmol) in Toluene (50 mL) was added B₂Pin₂ (2.65 g, 10.44 mmol), Pd(dppf)Cl₂ (508 mg, 0.696 mmol) and KOAc (1.71 g, 17.4 mmol) by one portion at room temperature. The reaction mixture was stirred 100° C. under N₂ atmosphere until all the starting material was translated, the reaction was monitored by TLC. After completion, the reaction solution sar filtrated and the filtration was washed with H₂O (20 mL×2). The mixture was dried under reduced pressure to obtain crude mixture, which was purified by silica gel column eluting with EtOAc/petroleum ether gradient from 0% to 10% to afford (S)-tert-butyl(4-(1-methoxyethyl)-3-(4,4,5,5-tetramethyl-1,3,2-dioxaborolan-2-yl)phenoxy)dimethylsilane (2.3 g, 84%) as an light green oil.

¹H NMR (400 MHz, DMSO-d6) δ 7.24 (d, J=8.4 Hz, 1H), 6.98 (d, J=2.6 Hz, 1H), 6.90 (dd, J=8.4, 2.6 Hz, 1H), 4.74 (q, J=6.2 Hz, 1H), 3.04 (s, 3H), 1.35-1.14 (m, 15H), 0.92 (s, 9H), 0.15 (s, 6H).

### Step 4. Synthesis of tert-butyl ((6³S,4S)-25-(benzyloxy)-1²-(5-((tert-butyldimethylsilyl)oxy)-2-((S)-1-methoxyethyl)phenyl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate

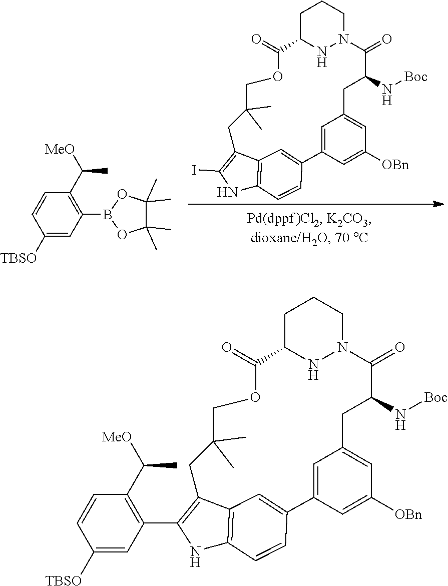 [SMILES: CO[C@@H](C)c1ccc(O[Si](C)(C)C(C)(C)C)cc1B1OC(C)(C)C(C)(C)O1 ; CC1(C)COC(=O)[C@@H]2CCCN(N2)C(=O)[C@@H](NC(=O)OC(C)(C)C)Cc2cc(OCc3ccccc3)cc(c2)-c2ccc3[nH]c(I)c(c3c2)C1 ; CO[C@@H](C)c1ccc(O[Si](C)(C)C(C)(C)C)cc1-c1[nH]c2ccc3cc2c1CC(C)(C)COC(=O)[C@@H]1CCCN(N1)C(=O)[C@@H](NC(=O)OC(C)(C)C)Cc1cc(OCc2ccccc2)cc-3c1]

To a solution of (S)-tert-butyl(4-(1-methoxyethyl)-3-(4,4,5,5-tetramethyl-1,3,2-dioxaborolan-2-yl)phenoxy)dimethylsilane (1.14 g, 2.91 mmol) and tert-butyl ((6³S,4S)-25-(benzyloxy)-1²-iodo-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (1.28 g, 1.616 mmol) in mixtured solvents dioxane/H₂O (50 mL, V:V=4:1) was added Pd(dppf)Cl₂ (118.2 g, 0.162 mmol) and K₂CO₃ (55.6 mg, 4.04 mmol) by one portion at room temperature under N₂ atmosphere. The reaction mixture was stirred at at 70° C. until the reaction was completed, the reaction was monitored by LCMS. After completion, the reaction was filtrated and the filtration was washed with H₂O (20 mL×2). The mixture was dried under reduced pressure to obtain tert-butyl ((6³S,4S)-2⁵-(benzyloxy)-1²-(5-((tert-butyldimethylsilyl)oxy)-2-((S)-1-methoxyethyl)phenyl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (2.4 g crude) as an brown solid, which was directly used for the next step without further purification.

LCMS (ESI) calcd. for C₅₄H₇₀N₄O₈Si [M-TBS+H]⁺ m/z 817.41, found: 817.8

### Step 5. Synthesis of tert-butyl ((6³S,4S)-2⁵-(benzyloxy)-1²-(5-hydroxy-2-((S)-1-methoxyethyl)phenyl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate

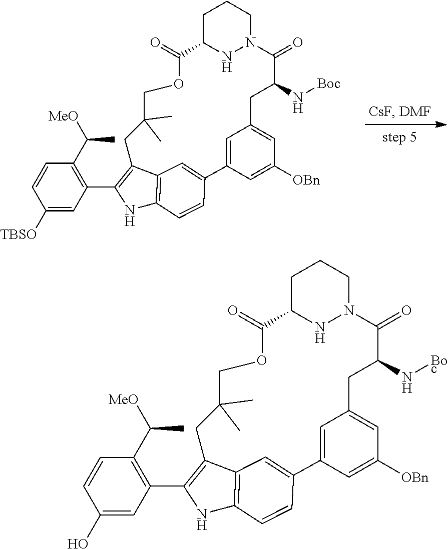 [SMILES: CO[C@@H](C)c1ccc(O[Si](C)(C)C(C)(C)C)cc1-c1[nH]c2ccc3cc2c1CC(C)(C)COC(=O)[C@@H]1CCCN(N1)C(=O)[C@@H](NC(=O)OC(C)(C)C)Cc1cc(OCc2ccccc2)cc-3c1 ; CO[C@@H](C)c1ccc(O)cc1-c1[nH]c2ccc3cc2c1CC(C)(C)COC(=O)[C@@H]1CCCN(N1)C(=O)[C@@H](NC(=O)OC(C)(C)C)Cc1cc(OCc2ccccc2)cc-3c1]

To a solution of tert-butyl ((6³S,4S)-2⁵-(benzyloxy)-1²-(5-((tert-butyldimethylsilyl)oxy)-2-((S)-1-methoxyethyl)phenyl)-10,10-dimethyl-5,7-dioxo-6¹,6²,6,6⁴,6⁵,6⁶-hexahydro-1 ¹H-8-oxa-1(5,3)-indola-6(1,3)-pyridazina-2(1,3)-benzenacycloundecaphane-4-yl)carbamate (2.4 g crude) in dry DMF (10 ml) was added CsF (720 mg, 4.50 mmol) by one batch, the mixture was stirred for 2 h room temperature, the reaction was monitored by LCMS. After completion, the reaction was diluted with saturated NaCl (20 mL) and was extracted with EtOAc (20 mL×4), the combined organic phase was washed with saturated NaCl (10 mL), then died over with Na₂SO₄, following with concentration under reduced pressure to obtain crude one, which was purified by silica gel column eluting with MeOH/DCM from 0% to 50% to afford tert-butyl ((6³S,4S)-2⁵-(benzyloxy)-1²-(5-hydroxy-2-((S)-1-methoxyethyl)phenyl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (1.2 g, 90% two steps) as a light yellow solid.

LCMS (ESI) calcd. for C₄₈H₅₆N₄O₈ [M+H]⁺ m/z 817.41, found: 817.9

### Step 6. Synthesis of 3-((6³S,4S)-2⁵-(benzyloxy)-4-((tert-butoxycarbonyl)amino)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-1²-yl)-4-((S)-1-methoxyethyl)phenyl trifluoromethanesulfonate

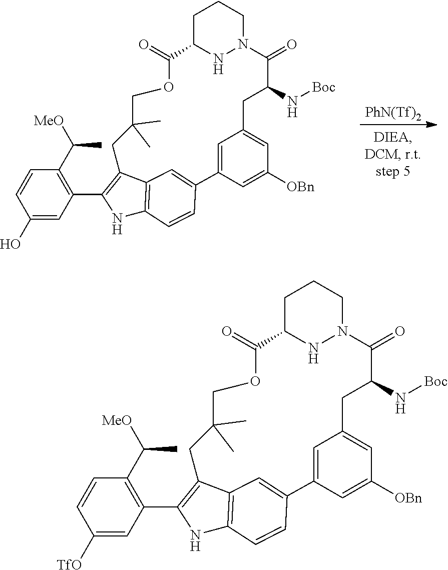 [SMILES: "PhN(Tf)2" ; CO[C@@H](C)c1ccc(O)cc1-c1[nH]c2ccc3cc2c1CC(C)(C)COC(=O)[C@@H]1CCCN(N1)C(=O)[C@@H](NC(=O)OC(C)(C)C)Cc1cc(OCc2ccccc2)cc-3c1 ; CO[C@@H](C)c1ccc(OS(=O)(=O)C(F)(F)F)cc1-c1[nH]c2ccc3cc2c1CC(C)(C)COC(=O)[C@@H]1CCCN(N1)C(=O)[C@@H](NC(=O)OC(C)(C)C)Cc1cc(OCc2ccccc2)cc-3c1]

To a solution of tert-butyl ((6³S,4S)-2⁵-(benzyloxy)-1²-(5-hydroxy-2-((S)-1-methoxyethyl)phenyl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (1.2 g, 1.46 mmol) in DCM (20 ml) was added DIEA (570 mg, 4.41 mmol) and PhN(Tf)₂ (755 mg, 2.94 mmol) by one batch at room temperature. The reaction solution was stirred at room temperature for 2 h, the reaction was monitored by LCMS. After completion, The reaction solution was washed with H₂O (10 mL×2), the organic layer was died over with Na₂SO₄, following with concentration under reduced pressure to obtain crude mixture, which was purified by silica gel column eluting with MeOH/DCM from 0% to 55% to afford 3-((6³S,4S)-2⁵-(benzyloxy)-4-((tert-butoxycarbonyl)amino)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-1²-yl)-4-((S)-1-methoxyethyl)phenyl trifluoromethanesulfonate (1.1 g, 79%) as a white solid.

LCMS (ESI) calcd. for C₄₉H₅₅F₃N₄O₁₀S [M+H]⁺ m/z 949.36, found: 949.1

### Step 7. Synthesis of tert-butyl ((6³S,4S)-2⁵-(benzyloxy)-1²-(2-((S)-1-methoxyethyl)-5-(4,4,5,5-tetramethyl-1,3,2-dioxaborolan-2-yl)phenyl)-10,10-dimethyl-5,7-dioxo-6¹,6²,6³,6⁴,6⁵,6⁶-hexahydro-1 ¹H-8-oxa-1(5,3)-indola-6(1,3)-pyridazina-2(1,3)-benzenacycloundecaphane-4-yl)carbamate

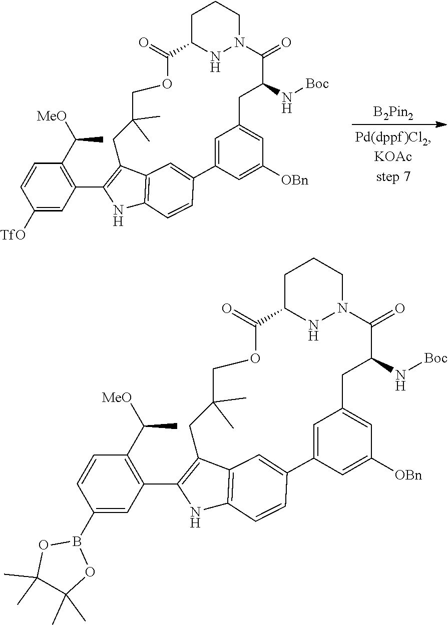 [SMILES: CO[C@@H](C)c1ccc(OS(=O)(=O)C(F)(F)F)cc1-c1[nH]c2ccc3cc2c1CC(C)(C)COC(=O)[C@@H]1CCCN(N1)C(=O)[C@@H](NC(=O)OC(C)(C)C)Cc1cc(OCc2ccccc2)cc-3c1 ; CO[C@@H](C)c1ccc(B2OC(C)(C)C(C)(C)O2)cc1-c1[nH]c2ccc3cc2c1CC(C)(C)COC(=O)[C@@H]1CCCN(N1)C(=O)[C@@H](NC(=O)OC(C)(C)C)Cc1cc(OCc2ccccc2)cc-3c1]

To a solution of 3-((6³S,4S)-2⁵-(benzyloxy)-4-((tert-butoxycarbonyl)amino)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-1²-yl)-4-((S)-1-methoxyethyl)phenyl trifluoromethanesulfonate (240 mg, 0.252 mmol) in Toluene (10 mL) was added B₂Pin₂ (128.4 mg, 0.504 mmol), Pd(dppt)Cl₂ (18.6 mg, 0.025 mmol) and KOAc (62.04 g, 0.63 mmol) by one portion at room temperature. The reaction mixture was stirred 100° C. under N₂ atmosphere until all the starting material was translated, the reaction was monitored by TLC. After completion, the reaction solution sar filtrated and the filtration was washed with H₂O (20 mL×2). The mixture was dried under reduced pressure to obtain crude mixture, which was purified by silica gel column eluting with EtOAc/petroleum ether gradient from 0% to 50% to afford tert-butyl ((6³S,4S)-2⁵-(benzyloxy)-1²-(2-((S)-1-methoxyethyl)-5-(4,4,5,5-tetramethyl-1,3,2-dioxaborolan-2-yl)phenyl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (145 mg, 62%) as a white solid.

LCMS (ESI) calcd. for C₅₄H₆₇BN₄O₉[M+H]⁺ m/z 927.50, found: 828.0

### Step 8. Synthesis of tert-butyl ((6³S,4S)-2⁵-(benzyloxy)-1¹-ethyl-1²-(2-((S)-1-methoxyethyl)-5-(4,4,5,5-tetramethyl-1,3,2-dioxaborolan-2-yl)phenyl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate

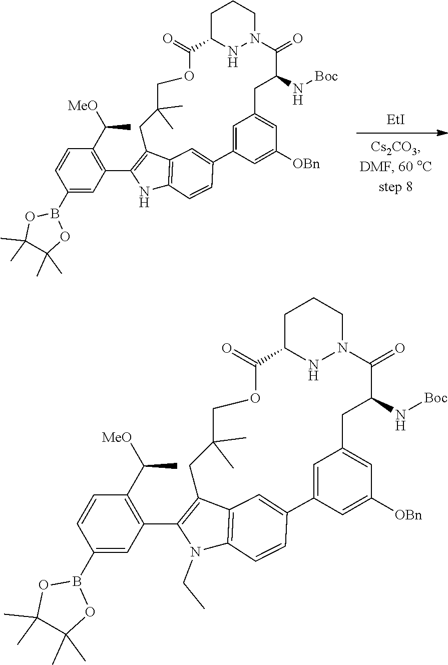 [SMILES: "EtI" ; CO[C@@H](C)c1ccc(B2OC(C)(C)C(C)(C)O2)cc1-c1[nH]c2ccc3cc2c1CC(C)(C)COC(=O)[C@@H]1CCCN(N1)C(=O)[C@@H](NC(=O)OC(C)(C)C)Cc1cc(OCc2ccccc2)cc-3c1 ; CCn1c(-c2cc(B3OC(C)(C)C(C)(C)O3)ccc2[C@H](C)OC)c2c3cc(ccc31)-c1cc(cc(OCc3ccccc3)c1)C[C@H](NC(=O)OC(C)(C)C)C(=O)N1CCC[C@H](N1)C(=O)OCC(C)(C)C2]

To a solution of tert-butyl ((6³S,4S)-2⁵-(benzyloxy)-1²-(2-((S)-1-methoxyethyl)-5-(4,4,5,5-tetramethyl-1,3,2-dioxaborolan-2-yl)phenyl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (100 mg, 0.108 mmol) in dry DMF (2 mL) was added EtI (168 mg, 1.08 mmol) and Cs₂CO₃ (351.6 mg, 1.08 mmol) under N₂ atmosphere protection. The reaction solution was stirred at room temperature under N₂ atmosphere overnight and the reaction was monitored by LCMS. After completion, the reaction solution was diluted with NaHCO₃ (10 mL) extracted with EtOAc (15 mL×4), the combined organic phase was washed with saturated NaCl (10 mL), then died over with Na₂SO₄, following with concentration under reduced pressure to obtain crude mixture, which was purified by silica gel column eluting with EtOAc/petroleum ether gradient from 0% to 40% to obtain tert-butyl ((6³S,4S)-2⁵-(benzyloxy)-1¹-ethyl-1²-(2-((S)-1-methoxyethyl)-5-(4,4,5,5-tetramethyl-1,3,2-dioxaborolan-2-yl)phenyl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (65 mg, 63%) as a white solid.

LCMS (ESI) calcd. for C₅₇H₇₁tBN₄O₉[M+H]⁺ m/z 955.53, found: 955.9

### Step 9. Synthesis of (6³S,4S)-4-amino-2⁵-(benzyloxy)-1¹-ethyl-1²-(2-((S)-1-methoxyethyl)-5-(4,4,5,5-tetramethyl-1,3,2-dioxaborolan-2-yl)phenyl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione

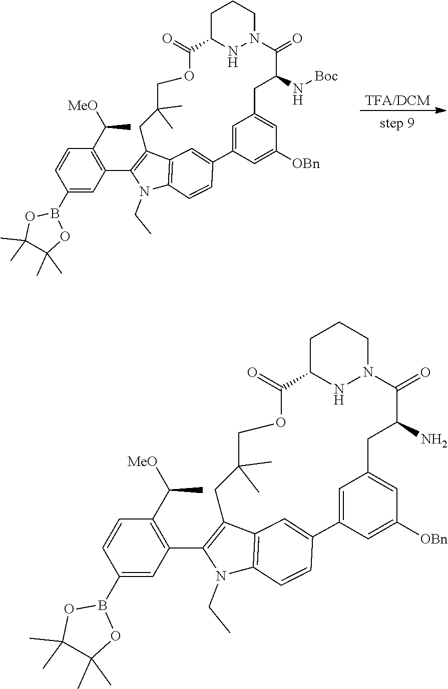 [SMILES: CCn1c(-c2cc(B3OC(C)(C)C(C)(C)O3)ccc2[C@H](C)OC)c2c3cc(ccc31)-c1cc(cc(OCc3ccccc3)c1)C[C@H](NC(=O)OC(C)(C)C)C(=O)N1CCC[C@H](N1)C(=O)OCC(C)(C)C2 ; "TFA/DCM" ; CCn1c(-c2cc(B3OC(C)(C)C(C)(C)O3)ccc2[C@H](C)OC)c2c3cc(ccc31)-c1cc(cc(OCc3ccccc3)c1)C[C@H](N)C(=O)N1CCC[C@H](N1)C(=O)OCC(C)(C)C2]

A solution of tert-butyl ((6³S,4S)-2⁵-(benzyloxy)-1¹-ethyl-1²-(2-((S)-1-methoxyethyl)-5-(4,4,5,5-tetramethyl-1,3,2-dioxaborolan-2-yl)phenyl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (40 mg, 0.041 mmol) in premixed solvents TFA/DCM (2.5 mL, V:V=1:4) was stirred at room temperature for 20 mins, the reaction was monitored by LCMS. After completion, The reaction was concentrated to obtain the crude mixture, which was purified by chromatography on silica gel (MeOH/DCM=0-8%) to obtain (6³S,4S)-4-amino-2⁵-(benzyloxy)-1¹-ethyl-1²-(2-((S)-1-methoxyethyl)-5-(4,4,5,5-tetramethyl-1,3,2-dioxaborolan-2-yl)phenyl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (26 mg, 74%) as a light yellow solid.

LCMS (ESI) calcd. for C₅₁H₆₃BN₄O₇[M+H]⁺ m/z 855.48, found: 855.8

### Step 10. Synthesis of methyl (3S,4R)-3-(((2S)-1-(((6³S,4S)-2⁵-(benzyloxy)-1¹-ethyl-1²-(2-((S)-1-methoxyethyl)-5-(4,4,5,5-tetramethyl-1,3,2-dioxaborolan-2-yl)phenyl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)carbamoyl)-4-methylpyrrolidine-1-carboxylate

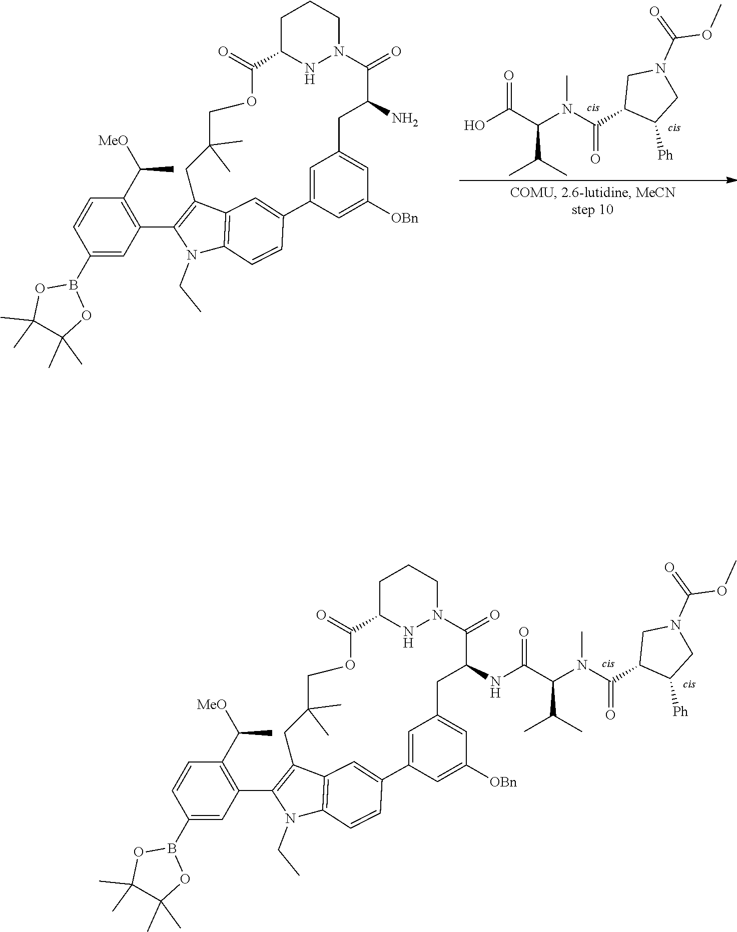

To a solution of cis mixture of N-((3S,4S)-1-(methoxycarbonyl)-4-phenylpyrrolidine-3-carbonyl)-N-methyl-L-valine (10.14 mg, 0.028 mmol) and (6³S,4S)-4-amino-2⁵-(benzyloxy)-1-ethyl-1²-(2-((S)-1-methoxyethyl)-5-(4,4,5,5-tetramethyl-1,3,2-dioxaborolan-2-yl)phenyl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (20 mg, 0.024 mmol) in MeCN (1 mL) was added 2,6-lutidine (5.04 mg, 0.47 mmol) and COMU (12.1 mg, 0.028 mmol) by one portion. The reaction was stirred at room temperature for 2 h. the reaction was monitored by LCMS. After completion, The reaction was purified by silica gel chromatography eluting with MeOH/DCM (from 0% to 5%) to afford methyl (3S,4S)-3-(((2S)-1-(((6³S,4S)-2⁵-(benzyloxy)-1¹-ethyl-1²-(2-((S)-1-methoxyethyl)-5-(4,4,5,5-tetramethyl-1,3,2-dioxaborolan-2-yl)phenyl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)carbamoyl)-4-phenylpyrrolidine-1-carboxylate (15 mg, 52%) as a white solid.

### Step 11. Synthesis of (3-((6³S,4S)-2⁵-(benzyloxy)-1¹-ethyl-4-((S)-2-((3S,4R)-1-(methoxycarbonyl)-N,4-dimethylpyrrolidine-3-carboxamido)-3-methylbutanamido)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-1²-yl)-4-((S)-1-methoxyethyl)phenyl)boronic acid

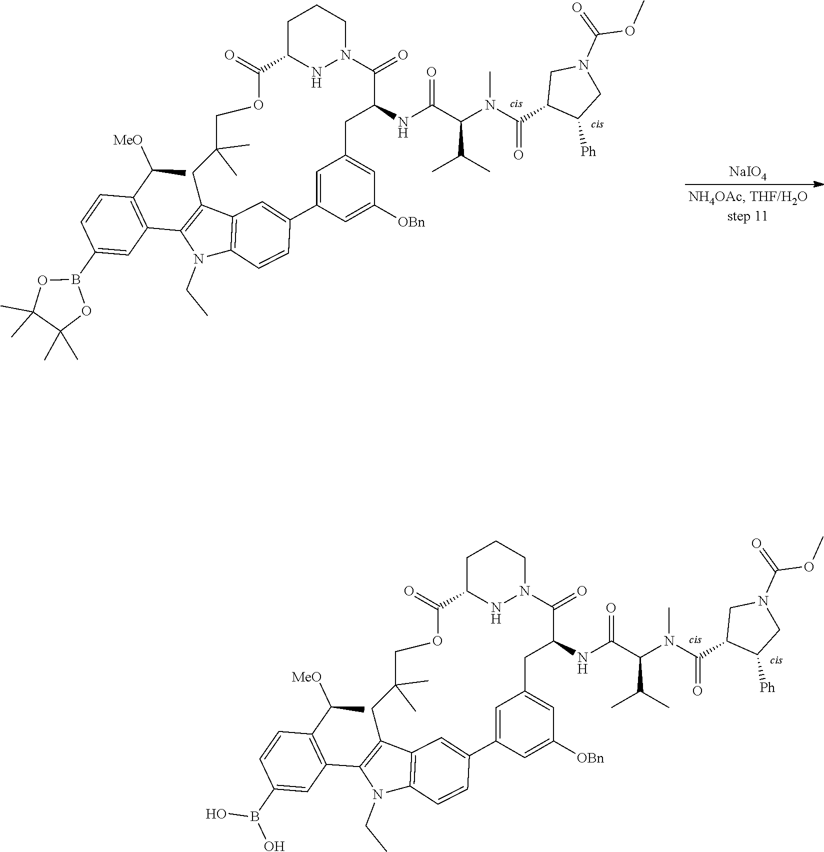

To a stirred solution of methyl (3S,4S)-3-(((2S)-1-(((6³S,4S)-2⁵-(benzyloxy)-1¹-ethyl-1²-(2-((S)-1-methoxyethyl)-5-(4,4,5,5-tetramethyl-1,3,2-dioxaborolan-2-yl)phenyl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)carbamoyl)-4-phenylpyrrolidine-1-carboxylate (15 mg, 0.012 mmol) in mixtured solvents of (THF/H₂O, V:V=1:1, 4 mL) was added NH4OAc (5.78 mg, 0.963 mmol) and NaIO₄ (20.63 mg, 0.963 mmol) at room temperature. The reaction was stirred at room temperature for 2 h, the reaction was monitored by LCMS. After completion, The reaction was purified by silica gel chromatography eluting with MeOH/DCM (from 0% to 8%) to afford (3-((6³S,4S)-2⁵-(benzyloxy)-1¹-ethyl-4-((S)-2-((3S,4R)-1-(methoxycarbonyl)-N,4-dimethylpyrrolidine-3-carboxamido)-3-methylbutanamido)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-1²-yl)-4-((S)-1-methoxyethyl)phenyl)boronic acid (5 mg, 37%) as a white solid.

LCMS (ESI) calcd. for C₅₉H₇₅BN₆O₁[M+H]⁺ m/z 1055.56, found: 1056.9

### Step 12. Synthesis of (3-((6³S,4S)-11-ethyl-2⁵-hydroxy-4-((S)-2-((3S,4R)-1-(methoxycarbonyl)-N,4-dimethylpyrrolidine-3-carboxamido)-3-methylbutanamido)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-1²-yl)-4-((S)-1-methoxyethyl)phenyl)boronic acid

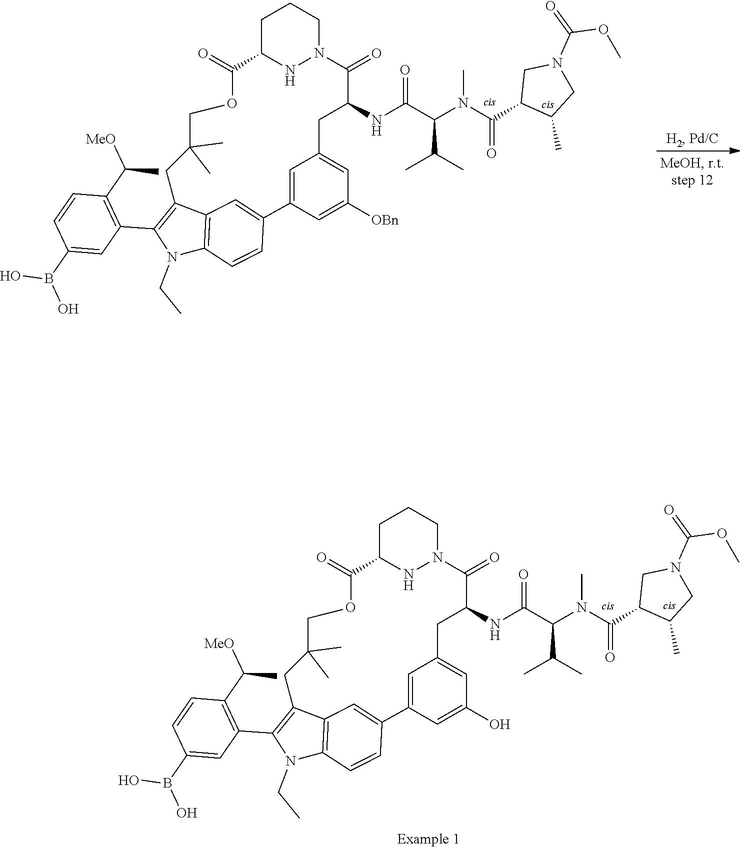 [SMILES: CCn1c(-c2cc(B(O)O)ccc2[C@H](C)OC)c2c3cc(ccc31)-c1cc(cc(OCc3ccccc3)c1)C[C@H](NC(=O)[C@H](C(C)C)N(C)C(=O)[C@@H]1CN(C(=O)OC)C[C@@H]1C)C(=O)N1CCC[C@H](N1)C(=O)OCC(C)(C)C2 ; "H2, Pd/C" ; CCn1c(-c2cc(B(O)O)ccc2[C@H](C)OC)c2c3cc(ccc31)-c1cc(O)cc(c1)C[C@H](NC(=O)[C@H](C(C)C)N(C)C(=O)[C@@H]1CN(C(=O)OC)C[C@@H]1C)C(=O)N1CCC[C@H](N1)C(=O)OCC(C)(C)C2]

To a solution of (3-((6³S,4S)-2⁵-(benzyloxy)-1¹-ethyl-4-((S)-2-((3S,4R)-1-(methoxycarbonyl)-N,4-dimethylpyrrolidine-3-carboxamido)-3-methylbutanamido)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-1²-yl)-4-((S)-1-methoxyethyl)phenyl)boronic acid (5 mg, 0.0045 mmol) in MeOH (2 mL) was added 10% Pd/C (1 mg) and the reaction was stirred under H₂ atmosphere (balloon) for 4 h. The reaction was monitored by LCMS. After completion, the reaction solution was concentrated and diluted with DCM (10 mL), then filtered. The filtration was concentrated to obtain crude mixture, which was further purified by silica gel chromatography eluting with MeOH/DCM (from 0% to 5%) to obtain (3-((6³S,4S)-1¹-ethyl-2⁵-hydroxy-4-((S)-2-((3S,4R)-1-(methoxycarbonyl)-N,4-dimethylpyrrolidine-3-carboxamido)-3-methylbutanamido)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-1²-yl)-4-((S)-1-methoxyethyl)phenyl)boronic acid (0.5 mg, 12%) as a white solid.

LCMS (ESI) calcd. for C52H₆₉BN₆O₁₁ [M+H]⁺ m/z 965.51 found: 965.8

### Example 2

### (2R,3R)—N-((2S)-1-(((6³S,4S)-1²-(5-(dimethylphosphoryl)-2-((S)-1-methoxyethyl)phenyl)-1¹-ethyl-2⁵-hydroxy-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-N-methyl-3-phenyltetrahydrofuran-2-carboxamide

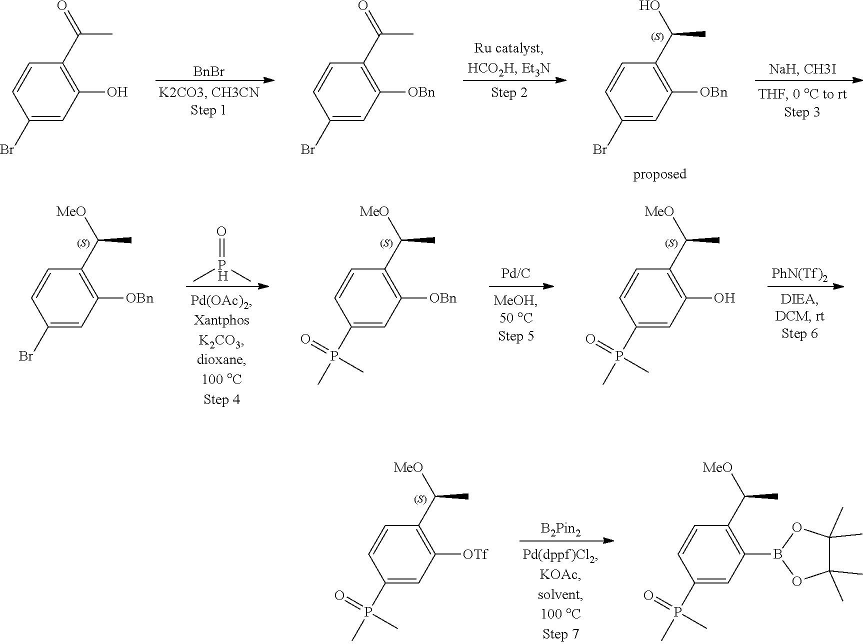

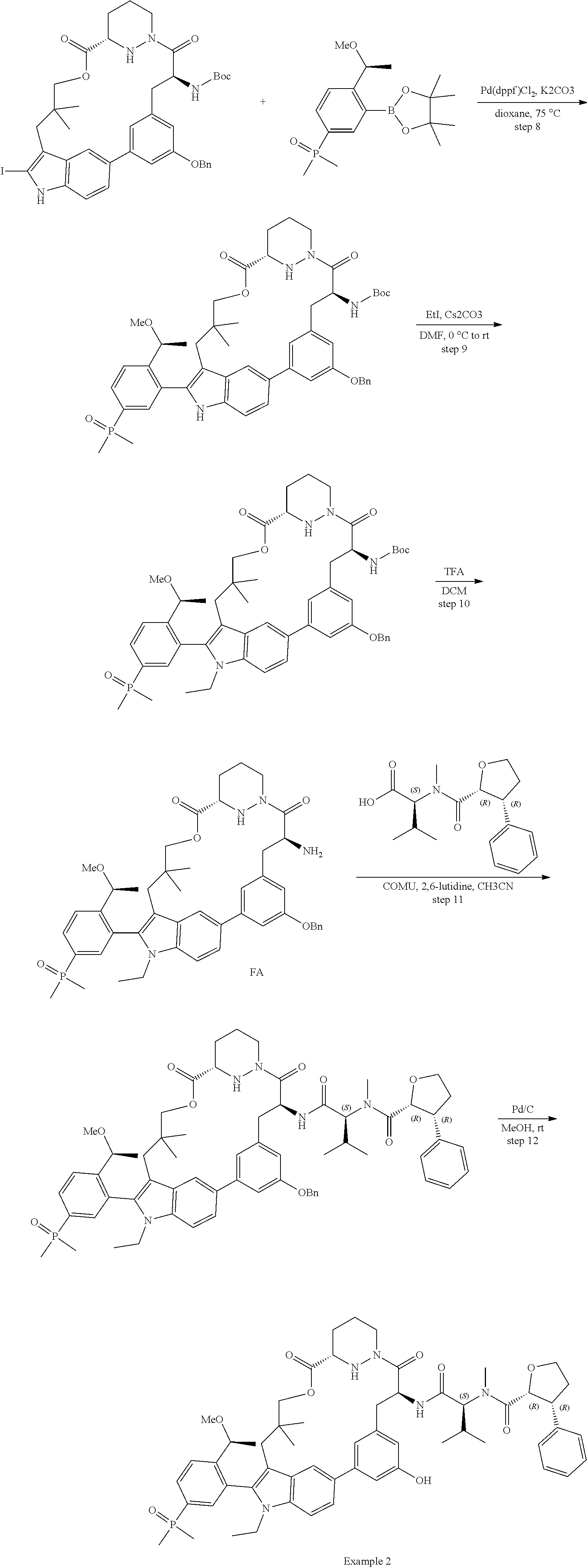

### Step 1. Synthesis of 1-(2-(benzyloxy)-4-bromophenyl)ethan-1-one

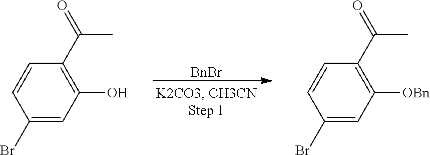

To a stirred solution of 1-(4-bromo-2-hydroxyphenyl)ethan-1-one (25 g, 116.84 mmol) in CH₃CN (500 mL) were added K₂CO₃ (32.2 g, 233.69 mmol) and BnBr (39.97 g, 233.69 mmol) at 0-10° C. The resulting mixture was heated to 80° C. and stirred at for 12 h. The reaction mixture was concentrated to give the residue. The residue was suspended in H₂O (500 mL) and extracted with EtOAc (2*200 mL). The organic layers were combined, washed with brine, dried over anhydrous Na₂SO₄ and concentrated to give the crude product, which was purified by silica gel column (eluting with 0-30% EtOAc in petroleum ether) to give 1-(2-(benzyloxy)-4-bromophenyl)ethan-1-one (30 g, 84.4%) as a white solid.

LCMS (ESI) calcd. for C₁₅H₁₃BrO₂ [M+H]⁺ m/z 304.01, found: 305.1

### Step 2. Synthesis of (S)-1-(2-(benzyloxy)-4-bromophenyl)ethan-1-ol

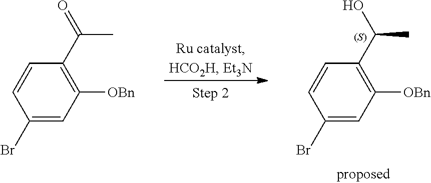

To a stirred solution of Et₃N (20.9 g, 28.7 mL, 206.44 mmol) was added FA (24.4 g, 20 mL, 530.85 mmol) at 0-5° C. The resulting mixture was warmed to rt. Then 1-(2-(benzyloxy)-4-bromophenyl)ethan-1-one (30 g, 98.31 mmol) and RuCl[(S,S)-TsDPEN](mesitylene) (624.4 mg, 0.98 mmol) was added into the above solution at rt. The resulting mixture was stirred at 60° C. under N₂ for 67 h. The reaction mixture was diluted with H₂O (250 mL) and acidified to pH=5-6 with 2 N HCl. The resulting mixture was extracted with EtOAc (200 mL). The organic layer was separated, dried over anhydrous Na₂SO₄ and concentrated to give the crude product. The crude product was purified by silica gel column (eluting with 0-20% EtOAc in petroleum ether) to afford (S)-1-(2-(benzyloxy)-4-bromophenyl)ethan-1-ol (25.6 g, 84.7%) as a white solid.

LCMS (ESI) calcd. for C₁₅H₁₅BrO₂ [M-17]⁺ m/z 306.03, found: 289.03

### Step 3. Synthesis of (S)-2-(benzyloxy)-4-bromo-1-(1-methoxyethyl)benzene

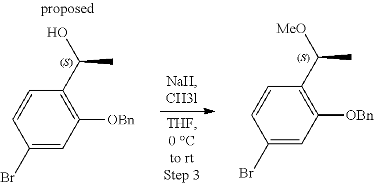

To a stirred solution of (S)-1-(2-(benzyloxy)-4-bromophenyl)ethan-1-ol (25.6 g, 83.66 mmol) in THF (380 mL) was added NaH (60%, 8.4 g, 209.15 mmol) portion wisely at 0° C.-5° C. The resulting mixture was stirred at this temperature for 30 min. Then CH₃I (35.6 g, 15.6 mL, 250.98 mmol) was added into the above solution. The resulting mixture was warmed to rt and stirred at the same temperature for 17 h. The reaction mixture was quenched with H₂O (150 mL) and then extracted with EtOAc (300 mL). The organic layer was separated, dried over anhydrous Na₂SO₄ and concentrated to give the crude product, which was purified by silica gel column (eluting with 0-10% EtOAc in petroleum ether) to afford (S)-2-(benzyloxy)-4-bromo-1-(1-methoxyethyl)benzene (24.6 g, 92%) as a pale yellow solid.

### Step 4. Synthesis of (S)-(3-(benzyloxy)-4-(1-methoxyethyl)phenyl)dimethylphosphine oxide

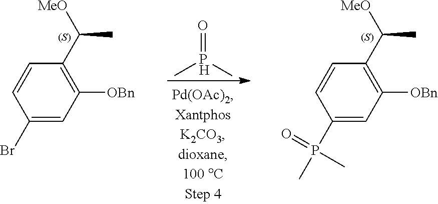

To a stirred solution of (S)-2-(benzyloxy)-4-bromo-1-(1-methoxyethyl)benzene (1.83 g, 5.70 mmol), dimethylphosphine oxide (2.22 g, 28.49 mmol) and K₂CO₃ (3.14 g, 22.79 mmol) in dioxane (30 ml) were added Pd(OAc)₂ (128 mg, 0.57 mmol) and Xantphos (659 mg, 1.14 mmol) at rt. The resulting mixture was stirred at 110° C. under N₂ for 17 h. The reaction mixture was concentrated to give the crude product, which was purified by silica gel column (eluting with 0-10% methanol in dichloromethane) to afford crude (S)-(3-(benzyloxy)-4-(1-methoxyethyl)phenyl)dimethylphosphine oxide (1.8 g, 98%) as a wine solid.

LCMS (ESI) calcd. for C₁₈H₂₃O₃P [M+H]⁺ m/z 318.14, found: 319.14

### Step 5. Synthesis of (S)-(3-hydroxy-4-(1-methoxyethyl)phenyl)dimethylphosphine oxide

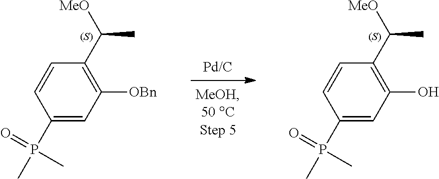

To a stirred solution of (S)-(3-(benzyloxy)-4-(1-methoxyethyl)phenyl)dimethylphosphine oxide (1.8 g, 5.66 mmol) in MeOH (40 ml) was added Pd/C (10%, 180 mg) at rt. The resulting mixture was stirred at 50° C. under H₂ (1 atm) for 16 h. The reaction mixture was filtered through a pad of celite and the filtrate was concentrated to give crude (S)-(3-hydroxy-4-(1-methoxyethyl)phenyl)dimethylphosphine oxide (1.26 g, 97%) as a grey solid.

LCMS (ESI) calcd. for C₁₁H₁₇O₃P [M+H]⁺ m/z 228.09, found: 229.09

### Step 6. Synthesis of (S)-5-(dimethylphosphoryl)-2-(1-methoxyethyl)phenyl trifluoromethanesulfonate

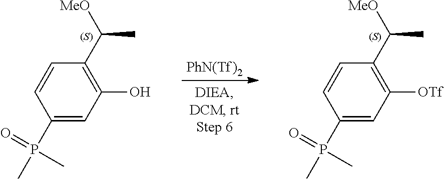

To a stirred solution of crude (S)-(3-hydroxy-4-(1-methoxyethyl)phenyl)dimethylphosphine oxide (1.69 g, 7.41 mmol) in DCM (35 mL) was added DIEA (2.40 g, 18.53 mmol) at rt. The resulting mixture was cooled to 8-10° C., and then PhN(Tf)₂ (4.24 g, 11.86 mmol) was added portion wisely into above solution. The reaction mixture was warmed to rt and stirred at the same temperature for 5 h. The reaction mixture was diluted with H₂O (100 mL) and extracted with dichloromethane (4*25 mL). The organic layers were combined, dried over anhydrous Na₂SO₄ and concentrated to give the crude product. The crude product was purified by silica gel column (eluting with 0-10% methanol in dichloromethane) to afford (S)-5-(dimethylphosphoryl)-2-(1-methoxyethyl)phenyl trifluoromethanesulfonate (2.3 g, 85.9%) as an off-white solid.

LCMS (ESI) calcd. for C₁₂H₁₆F₃O₅PS [M+H]⁺ m/z 360.04, found: 361.04

### Step 7. Synthesis of (S)-(4-(1-methoxyethyl)-3-(4,4,5,5-tetramethyl-1,3,2-dioxaborolan-2-yl)phenyl)dimethylphosphine oxide

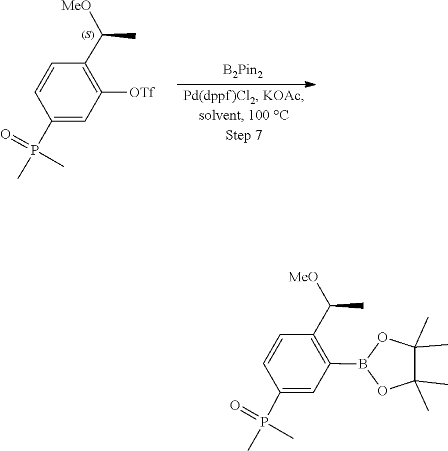

To a stirred suspension of (S)-5-(dimethylphosphoryl)-2-(1-methoxyethyl)phenyl trifluoromethanesulfonate (1.2 g, 3.33 mmol), (BPin)₂ (1.69 g, 6.67 mmol) and AcOK (981 mg, 10.0 mmol) in toluene (25 mL) was added Pd(dppf)Cl₂ (244 mg, 0.33 mmol) at rt. The resulting mixture was stirred at 100° C. under N₂ for 16 h. The reaction mixture was filtered through a pad of celite. The filtrate was concentrated to give the crude product, which was purified by reverse phase column chromatography (eluting with 0-40% acetonitrile in H2O (0.1% NH₄HCO₃)) to give (S)-(4-(1-methoxyethyl)-3-(4,4,5,5-tetramethyl-1,3,2-dioxaborolan-2-yl)phenyl)dimethylphosphine oxide (600 mg, 53%) as a grey solid.

LCMS (ESI) calcd. for C₁₇H₂₈BO₄P [M+H]⁺ m/z 338.18, found: 339.18

### Step 8. Synthesis of tert-butyl ((6³S,4S)-2⁵-(benzyloxy)-1²-(5-(dimethylphosphoryl)-2-((S)-1-methoxyethyl)phenyl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate

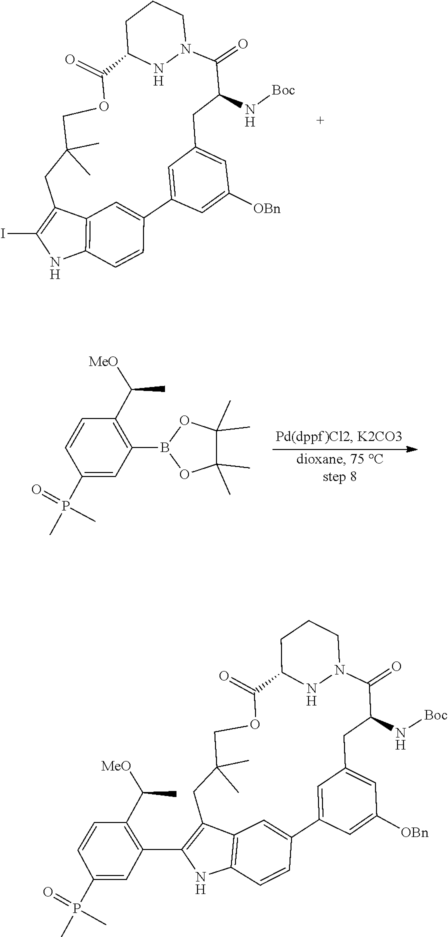 [SMILES: CC1(C)COC(=O)[C@@H]2CCCN(N2)C(=O)[C@@H](NC(=O)OC(C)(C)C)Cc2cc(OCc3ccccc3)cc(c2)-c2ccc3[nH]c(I)c(c3c2)C1 ; CO[C@@H](C)c1ccc(P(C)(C)=O)cc1B1OC(C)(C)C(C)(C)O1 ; CO[C@@H](C)c1ccc(P(C)(C)=O)cc1-c1[nH]c2ccc3cc2c1CC(C)(C)COC(=O)[C@@H]1CCCN(N1)C(=O)[C@@H](NC(=O)OC(C)(C)C)Cc1cc(OCc2ccccc2)cc-3c1]

To a stirred suspension of tert-butyl ((6³S,4S)-2⁵-(benzyloxy)-1²-iodo-10,10-dimethyl-5,7-dioxo-6¹,6²,6³,6⁴,6⁵,6⁶-hexahydro-1H-8-oxa-1(5,3)-indola-6(1,3)-pyridazina-2(1,3)-benzenacycloundecaphane-4-yl)carbamate (644 mg, 0.81 mmol), (S)-(4-(1-methoxyethyl)-3-(4,4,5,5-tetramethyl-1,3,2-dioxaborolan-2-yl)phenyl)dimethylphosphine oxide (550 mg, 1.63 mmol) and K₂CO₃ (336.7 mg, 2.44 mmol) in dioxane (13 mL) and H₂O (3.3 mL) was added Pd(dppf)Cl₂ at rt. The resulting mixture was stirred at 75° C. under N₂ 18 h. The reaction mixture was concentrated to give the crude product, which was purified by silica gel column (eluting with 0-10% methanol in dichloromethane) to afford crude tert-butyl ((6³S,4S)-2⁵-(benzyloxy)-1²-(5-(dimethylphosphoryl)-2-((S)-1-methoxyethyl)phenyl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (805 mg, assumed 100%) as a wine solid.

LCMS (ESI) calcd. for C₅₀H₆₁N₄O₈P [M-100+H]⁺ m/z 876.42, found: 777.4

### Step 9. Synthesis of tert-butyl ((6³S,4S)-2⁵-(benzyloxy)-1²-(5-(dimethylphosphoryl)-2-((S)-1-methoxyethyl)phenyl)-1¹-ethyl-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate

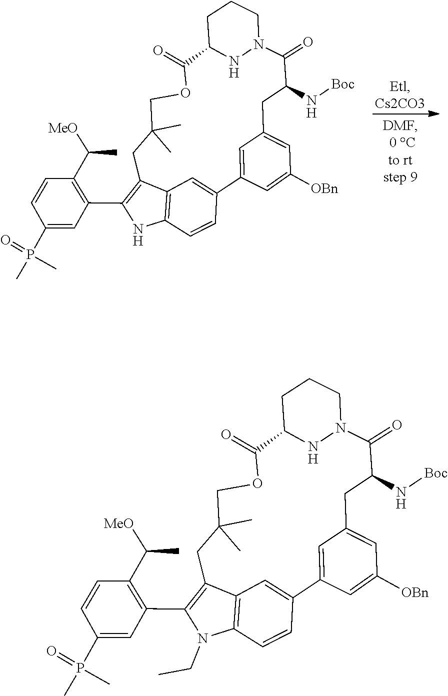 [SMILES: CO[C@@H](C)c1ccc(P(C)(C)=O)cc1-c1[nH]c2ccc3cc2c1CC(C)(C)COC(=O)[C@@H]1CCCN(N1)C(=O)[C@@H](NC(=O)OC(C)(C)C)Cc1cc(OCc2ccccc2)cc-3c1 ; CCn1c(-c2cc(P(C)(C)=O)ccc2[C@H](C)OC)c2c3cc(ccc31)-c1cc(cc(OCc3ccccc3)c1)C[C@H](NC(=O)OC(C)(C)C)C(=O)N1CCC[C@H](N1)C(=O)OCC(C)(C)C2]

To a stirred solution of crude tert-butyl ((6³S,4S)-2⁵-(benzyloxy)-1²-(5-(dimethylphosphoryl)-2-((S)-1-methoxyethyl)phenyl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (805 mg, 0.92 mmol) in DMF (20 mL) were added CS₂CO₃ (598 mg, 1.84 mmol) and EtI (287 mg, 1.84 mmol) at rt and the resulting mixture was stirred at the same temperature for 19 h. The reaction mixture was diluted with H₂O (200 mL). The suspension was filtered and the cake was washed with H₂O. The cake was collected and dried to give tert-butyl ((6³S,4S)-25-(benzyloxy)-1²-(5-(dimethylphosphoryl)-2-((S)-1-methoxyethyl)phenyl)-1¹-ethyl-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (685 mg, 82.4%) as a brown solid.

LCMS (ESI) calcd. for C₅₂H₆₅N₄O₈P [M-100+H]⁺ m/z 904.45, found: 805.5

### Step 10. Synthesis of (6³S,4S)-4-amino-2⁵-(benzyloxy)-1²-(5-(dimethylphosphoryl)-2-((S)-1-methoxyethyl)phenyl)-1¹-ethyl-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione

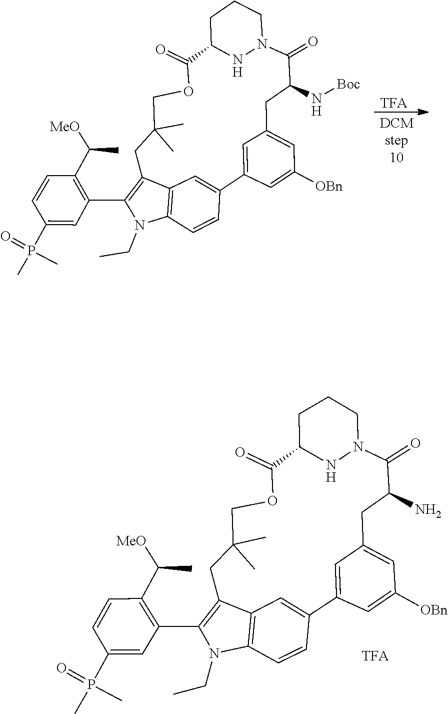 [SMILES: CCn1c(-c2cc(P(C)(C)=O)ccc2[C@H](C)OC)c2c3cc(ccc31)-c1cc(cc(OCc3ccccc3)c1)C[C@H](NC(=O)OC(C)(C)C)C(=O)N1CCC[C@H](N1)C(=O)OCC(C)(C)C2 ; CCn1c(-c2cc(P(C)(C)=O)ccc2[C@H](C)OC)c2c3cc(ccc31)-c1cc(cc(OCc3ccccc3)c1)C[C@H](N)C(=O)N1CCC[C@H](N1)C(=O)OCC(C)(C)C2]

To a stirred solution of tert-butyl ((6³S,4S)-2⁵-(benzyloxy)-1²-(5-(dimethylphosphoryl)-2-((S)-1-methoxyethyl)phenyl)-1¹-ethyl-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (685 mg, 0.76 mmol) in dichloromethane (7 mL) was added TFA (3.5 mL) dropwisely at rt and the resulting mixture was stirred at rt for 2 h. The reaction mixture was concentrated to give the residue, which was purified by reverse phase column chromatography (eluting with 0-60% acetonitrile in H₂O (0.5% FA)) to afford (6³S,4S)-4-amino-2⁵-(benzyloxy)-1²-(5-(dimethylphosphoryl)-2-((S)-1-methoxyethyl)phenyl)-1¹-ethyl-10,10-dimethyl-6¹,6²,6³,6⁴,6⁵,6⁶-hexahydro-1H-8-oxa-1(5,3)-indola-6(1,3)-pyridazina-2(1,3)-benzenacycloundecaphane-5,7-dione (125 mg, 18%) as a white solid of its FA salt.

LCMS (ESI) calcd. for C₄₇H₅₇N₄O₆P [M+H]⁺ m/z 804.40, found: 805.4

### Step 11. Synthesis of (2R,3R)—N-((2S)-1-(((6³S,4S)-2⁵-(benzyloxy)-1²-(5-(dimethylphosphoryl)-2-((S)-1-methoxyethyl)phenyl)-1¹-ethyl-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-N-methyl-3-phenyltetrahydrofuran-2-carboxamide

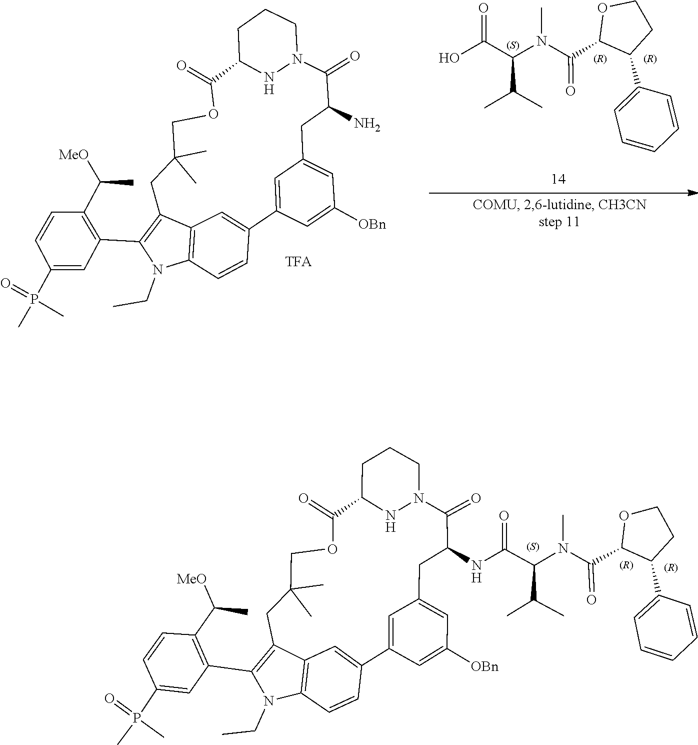

To a stirred solution of (6³S,4S)-4-amino-2⁵-(benzyloxy)-1²-(5-(dimethylphosphoryl)-2-((S)-1-methoxyethyl)phenyl)-1¹-ethyl-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (125 mg, 0.15 mmol) and N-methyl-N-((2R,3R)-3-phenyltetrahydrofuran-2-carbonyl)-L-valine (55 mg, 0.18 mmol) and 2,6-lutidine (19 mg, 0.18 mmol) in MeCN (5 mL) was added COMU (128 mg, 0.30 mmol) at rt and the resulting mixture was stirred at the same temperature for 15 h. The reaction mixture was concentrated to give the crude product, which was purified by silica gel column (eluting with 0-10% methanol in dichloromethane) to give crude (2R,3R)—N-((2S)-1-(((6³S,4S)-2⁵-(benzyloxy)-1²-(5-(dimethylphosphoryl)-2-((S)-1-methoxyethyl)phenyl)-1¹-ethyl-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-N-methyl-3-phenyltetrahydrofuran-2-carboxamide (120 mg, 73.2%) as a pale pink solid.

LCMS (ESI) calcd. for C₆₄H₇₈N₅O₉P [M+H]⁺ m/z 1091.55, found: 1092.5

### Synthesis of (2R,3R)—N-((2S)-1-(((6³S,4S)-1²-(5-(dimethylphosphoryl)-2-((S)-1-methoxyethyl)phenyl)-1¹-ethyl-2⁵-hydroxy-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-N-methyl-3-phenyltetrahydrofuran-2-carboxamide

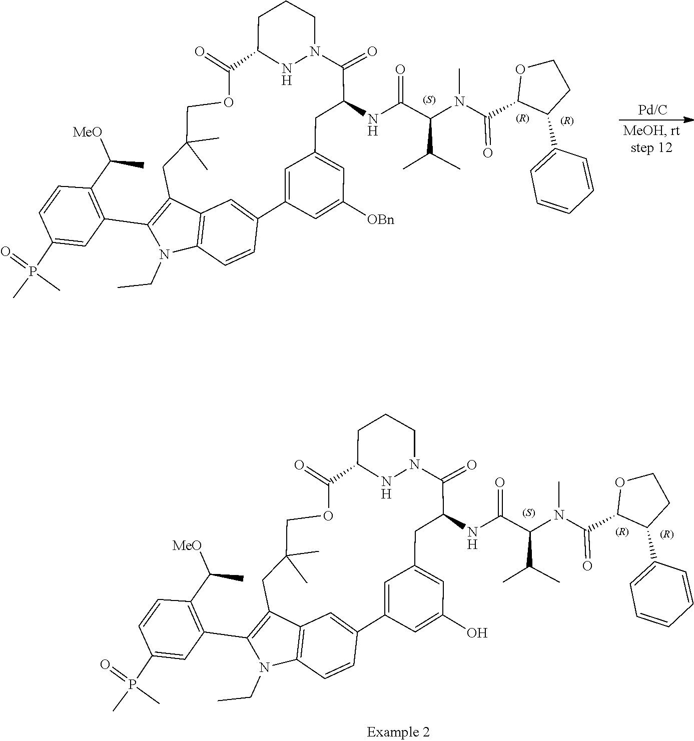

To a stirred solution of (2R,3R)—N-((2S)-1-(((6³S,4S)-2⁵-(benzyloxy)-1²-(5-(dimethylphosphoryl)-2-((S)-1-methoxyethyl)phenyl)-1¹-ethyl-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-N-methyl-3-phenyltetrahydrofuran-2-carboxamide (120 mg, 0.11 mmol) in MeOH (10 mL) was added Pd/C (10%, 12 mg) at rt. The resulting mixture was stirred at rt under H₂ (1 atm) for 16 h. The reaction mixture was filtered through a pad of celite. The filtrate was concentrated to give the crude product, which was purified by reverse phase column chromatography (eluting with 0-60% acetonitrile in H₂O (0.5% NH₄HCO₃)) to give (2R,3R)—N-((2S)-1-(((6³S,4S)-1²-(5-(dimethylphosphoryl)-2-((S)-1-methoxyethyl)phenyl)-1¹-ethyl-2⁵-hydroxy-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-N-methyl-3-phenyltetrahydrofuran-2-carboxamide (19 mg, 17.2%) as a white solid.

LCMS (ESI) calcd. for C₅₇H₇₂N₅O₉P [M+H]⁺ m/z 1001.51, found: 1002.5

### Example 3

### (2R,3R)—N-((2S)-1-(((6³S,4S)-1²-(5-(dimethylphosphorothioyl)-2-((S)-1-methoxyethyl)phenyl)-11-ethyl-2⁵-hydroxy-10,10-dimethyl-5,7-dioxo-6′,6²,63,64,65,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-N-methyl-3-phenyltetrahydrofuran-2-carboxamide

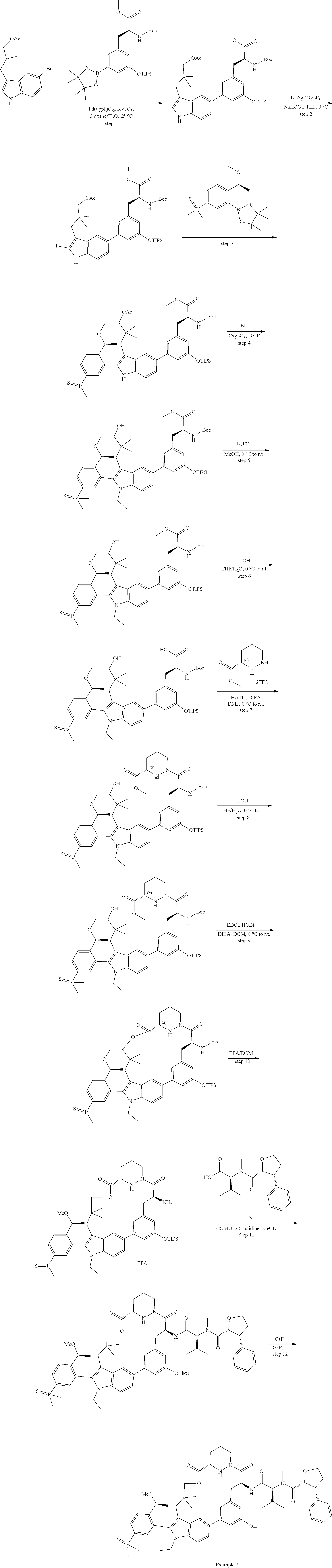

### Step 1. Synthesis of methyl (S)-3-(3-(3-(3-acetoxy-2,2-dimethylpropyl)-1H-indol-5-yl)-5-((triisopropylsilyl)oxy)phenyl)-2-((tert-butoxycarbonyl)amino)propanoate

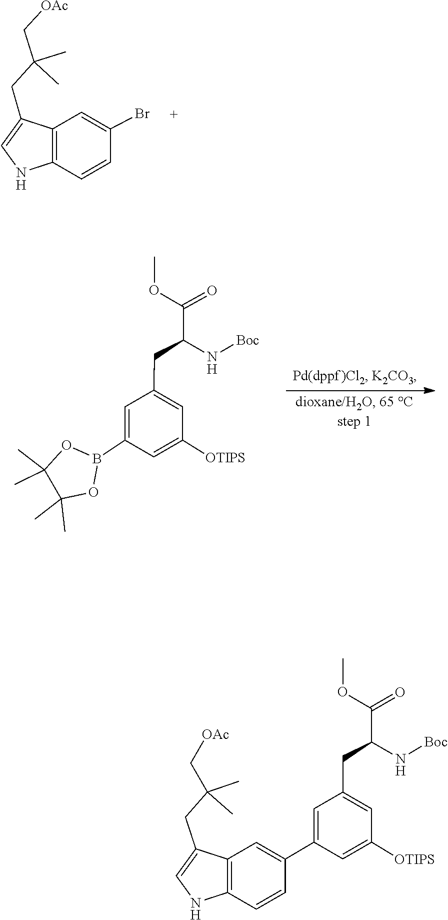

To a stirred solution of 3-(5-bromo-1H-indol-3-yl)-2,2-dimethylpropyl acetate (560 mg, 1.73 mmol) and methyl (S)-3-(3-(benzyloxy)-5-(4,4,5,5-tetramethyl-1,3,2-dioxaborolan-2-yl)phenyl)-2-((tert-butoxycarbonyl)amino)propanoate (1.6 g, 2.77 mmol) in pre-mixtured solvents dioxane/H₂O (10 mL, 4:1, V:V) was added Pd(dppf)Cl₂ (127 mg, 0.17 mmol) and K₂CO₃ (718 mg, 5.20 mmol). The reaction mixture was stirred at 75° C. for 5 h under N₂ atmosphere and the reaction was monitored by LCMS. After completion, the reaction solution was concentrated under reduced pressure to remove dioxane, then the residual was diluted with H₂O and extracted with EtOAc (20 mL×3). The combined organic phase was washed with saturated NaCl (20 mL), then dried over Na₂SO₄, following with concentration under reduced pressure to obtain crude product which was purified by silica gel column eluting with EtOAc/petroleum ether from 0% to 50% to afford methyl (S)-3-(3-(3-(3-acetoxy-2,2-dimethylpropyl)-1H-indol-5-yl)-5-((triisopropylsilyl)oxy)phenyl)-2-((tert-butoxycarbonyl)amino)propanoate (1.1 g, 910%) as a light yellow gum.

LCMS (ESI) calcd. for C₃₉H₅₈N₂O₇Si [M+H]⁺ m/z 695.40, found: 695.6

### Step 2. Synthesis of methyl (S)-3-(3-(3-(3-acetoxy-2,2-dimethylpropyl)-2-iodo-1H-indol-5-yl)-5-((triisopropylsilyl)oxy)phenyl)-2-((tert-butoxycarbonyl)amino)propanoate

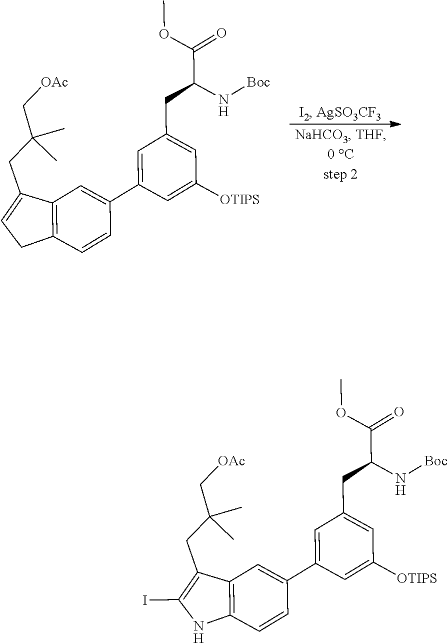

To a stirred suspension of methyl (S)-3-(3-(3-(3-acetoxy-2,2-dimethylpropyl)-2-iodo-1H-indol-5-yl)-5-((triisopropylsilyl)oxy)phenyl)-2-((tert-butoxycarbonyl)amino)propanoate (1.1 g, 1.59 mmol) and NaHCO₃ (160 mg, 1.90 mmol) in THF (10 mL) under ice-water bath was added AgSO₃CF₃ (489 mg, 1.90 mmol) in THF (5 mL) and I₂ (402 mg, 1.59 mmol) in THF (5 mL) by dropwise to the reaction mixture. The reaction mixture was stirred at 0° C. for 2 h under N₂ atmosphere and the reaction was monitored by LCMS. After completion, The reaction was quenched with saturated Na₂S₂O₃ (20 mL) and the resulting mixture was extracted with EtOAc (20 mL×2). The combined organic phase was washed with saturated NaCl (25 mL), then dried over Na₂SO₄, following with concentration under reduced pressure to obtain crude product which was purified by silica gel column eluting with EtOAc/petroleum ether from 0% to 40% to afford methyl (S)-3-(3-(3-(3-acetoxy-2,2-dimethylpropyl)-2-iodo-1H-indol-5-yl)-5-((triisopropylsilyl)oxy)phenyl)-2-((tert-butoxycarbonyl)amino)propanoate (820 mg, 62.8%) as a pale yellow foam.

LCMS (ESI) calcd. for C₃₉H57IN₂O₇Si [M+H]⁺ m/z 821.30, found: 821.6

### Step 3. Synthesis of methyl (S)-2-((tert-butoxycarbonyl)amino)-3-(3-(2-(5-(dimethylphosphorothioyl)-2-((S)-1-methoxyethyl)phenyl)-3-(3-hydroxy-2,2-dimethylpropyl)-1H-indol-5-yl)-5-((triisopropylsilyl)oxy)phenyl)propanoate

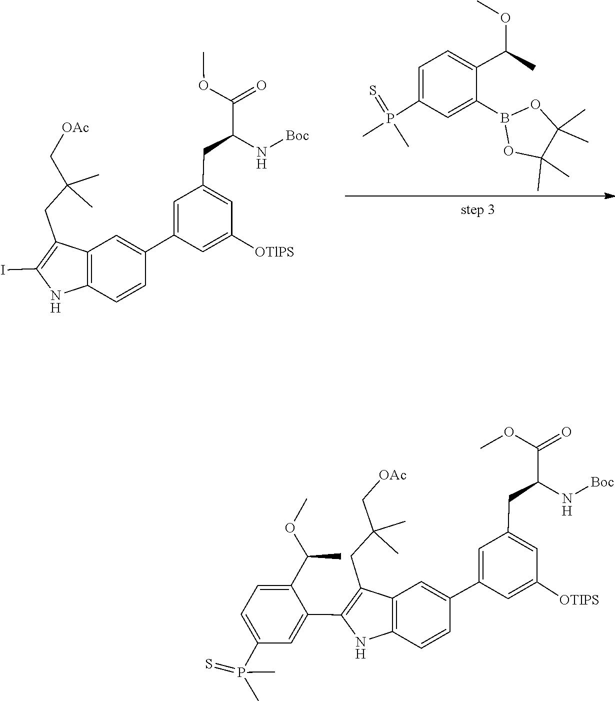

To a stirred solution of methyl (S)-3-(3-(3-(3-acetoxy-2,2-dimethylpropyl)-2-iodo-1H-indol-5-yl)-5-((triisopropylsilyl)oxy)phenyl)-2-((tert-butoxycarbonyl)amino)propanoate (700 mg, 0.85 mmol), (S)-(4-(1-methoxyethyl)-3-(4,4,5,5-tetramethyl-1,3,2-dioxaborolan-2-yl)phenyl)dimethylphosphine sulfide (483 mg, 1.36 mmol) and K₂CO₃ (353 mg, 2.56 mmol) in dioxane/H₂O (17.5 mL, V:V=4:1) was added Pd(dppf)Cl₂ (94 mg, 0.13 mmol) at rt. The reaction mixture was stirred at 100° C. under N₂ for 18 h. The reaction was monitored by LCMS. After completion, the reaction mixture was concentrated to give crude product, which was purified by silica gel column chromatography (eluting with 0-100% EtOAc in petroleum ether) to give methyl (S)-2-((tert-butoxycarbonyl)amino)-3-(3-(2-(5-(dimethylphosphorothioyl)-2-((S)-1-methoxyethyl)phenyl)-3-(3-hydroxy-2,2-dimethylpropyl)-1H-indol-5-yl)-5-((triisopropylsilyl)oxy)phenyl)propanoate (410 mg, 52.3%) as a brown solid.

LCMS (ESI) calcd. for C₅₀H₇₃N₂O₈PSSi [M+H]⁺ m/z 921.46, found: 921.7

### Step 4. Synthesis of methyl (S)-2-((tert-butoxycarbonyl)amino)-3-(3-(2-(5-(dimethylphosphorothioyl)-2-((S)-1-methoxyethyl)phenyl)-1-ethyl-3-(3-hydroxy-2,2-dimethylpropyl)-1H-indol-5-yl)-5-((triisopropylsilyl)oxy)phenyl)propanoate

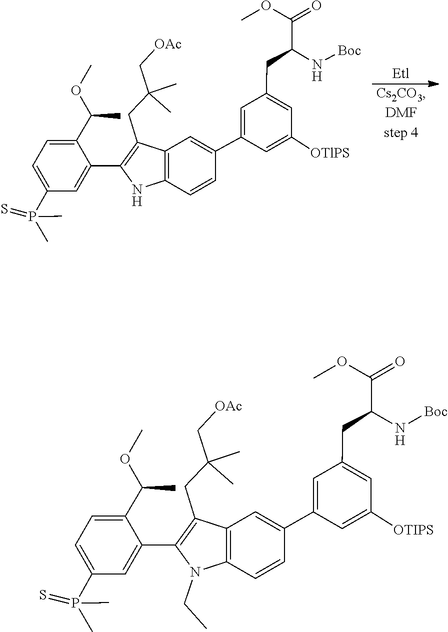

To a solution of methyl (S)-2-((tert-butoxycarbonyl)amino)-3-(3-(2-(5-(dimethylphosphorothioyl)-2-((S)-1-methoxyethyl)phenyl)-1-ethyl-3-(3-hydroxy-2,2-dimethylpropyl)-1H-indol-5-yl)-5-((triisopropylsilyl)oxy)phenyl)propanoate (410 mg, 0.45 mmol), in DMF (8 ml) were added Cs₂CO₃ (290 mg, 0.89 mmol) and EtI (139 mg, 0.89 mmol) at rt and the resulting mixture was stirred at the same temperature for 3 h. The reaction was monitored by LCMS. After completion, the reaction mixture was diluted with H₂O (50 mL) and extracted with EtOAc (15 mL×3). The combined organic phase was washed with saturated NaCl (50 mL), dried over Na₂SO₄, following with concentration under reduced pressure to obtain crude product which was purified by silica gel column (eluting with 0-40% EtOAc in petroleum ether) to afford methyl (S)-2-((tert-butoxycarbonyl)amino)-3-(3-(2-(5-(dimethylphosphorothioyl)-2-((S)-1-methoxyethyl)phenyl)-1-ethyl-3-(3-hydroxy-2,2-dimethylpropyl)-1H-indol-5-yl)-5-((triisopropylsilyl)oxy)phenyl)propanoate (270 mg, 63.2%) as a brown foam.

LCMS (ESI) calcd. for C₅₂H₇₇N₂O₇PSSi [M+H]⁺ m/z 949.49, found: 949.6

### Step 5. Synthesis of methyl (S)-2-((tert-butoxycarbonyl)amino)-3-(3-(2-(5-(dimethylphosphorothioyl)-2-((S)-1-methoxyethyl)phenyl)-1-ethyl-3-(3-hydroxy-2,2-dimethylpropyl)-1H-indol-5-yl)-5-((triisopropylsilyl)oxy)phenyl)propanoate

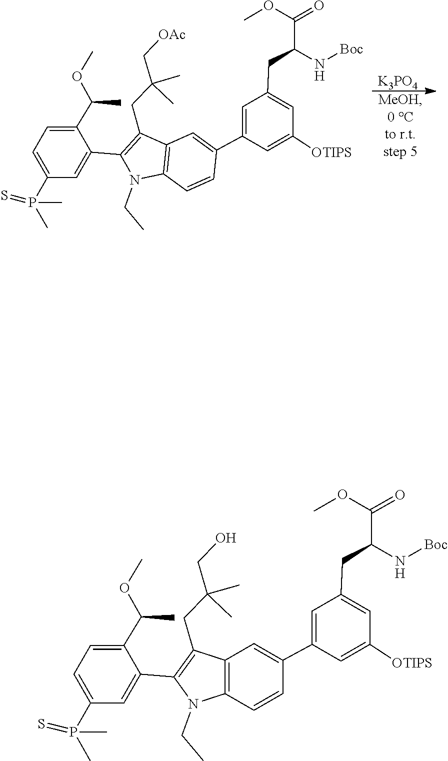

To a solution of methyl (S)-3-(3-(3-(3-acetoxy-2,2-dimethylpropyl)-2-(5-(dimethylphosphorothioyl)-2-((S)-1-methoxyethyl)phenyl)-1-ethyl-1H-indol-5-yl)-5-((triisopropylsilyl)oxy)phenyl)-2-((tert-butoxycarbonyl)amino)propanoate (270 mg, 0.28 mmol) in MeOH (225 mL) was added K₃PO₄ (181 mg, 0.85 mmol) at 0-10° C. The reaction solution was stirred at room temperature for 6 h. The reaction was monitored by LCMS. After completion, the reaction mixture was diluted with brine and extracted with EtOAc (2×20 mL). The organic layers were combined, dried over anhydrous Na₂SO₄ and concentrated to give the crude product. The crude product was purified by silica gel column chromatography (eluting with 0-45% EtOAc in petroleum ether) to give methyl (S)-2-((tert-butoxycarbonyl)amino)-3-(3-(2-(5-(dimethylphosphorothioyl)-2-((S)-1-methoxyethyl)phenyl)-1-ethyl-3-(3-hydroxy-2,2-dimethylpropyl)-1H-indol-5-yl)-5-((triisopropylsilyl)oxy)phenyl)propanoate (168 mg, 66.2%) as a white solid.

LCMS (ESI) calcd. for Cs₂H₇₇N₂O₇PSSi [M+H]⁺ m/z 907.48, found: 907.6

### Step 6. Synthesis of (S)-2-((tert-butoxycarbonyl)amino)-3-(3-(2-(5-(dimethylphosphorothioyl)-2-((S)-1-methoxyethyl)phenyl)-1-ethyl-3-(3-hydroxy-2,2-dimethylpropyl)-1H-indol-5-yl)-5-((triisopropylsilyl)oxy)phenyl)propanoic acid

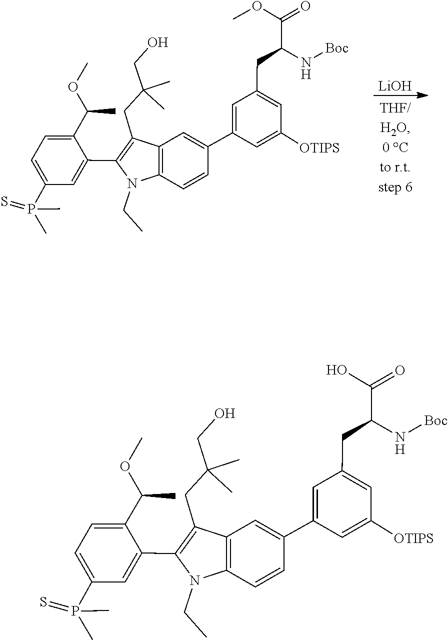

To a solution of methyl (S)-2-((tert-butoxycarbonyl)amino)-3-(3-(2-(5-(dimethylphosphorothioyl)-2-((S)-1-methoxyethyl)phenyl)-1-ethyl-3-(3-hydroxy-2,2-dimethylpropyl)-1H-indol-5-yl)-5-((triisopropylsilyl)oxy)phenyl)propanoate (160 mg, 0.18 mmol) in THF (3 mL) and H₂O (1.5 mL) at 0-10° C. was added LiOH (22 mg, 0.53 mmol) at 0-10° C. and the resulting mixture was stirred at the same temperature for 2 h. The reaction was monitored by LCMS. After completion, the reaction mixture was diluted with H₂O (20 mL) and acidified to pH=4-5 with 2 N HCl. The resulting mixture was extracted with DCM (2×15 mL). The organic layers were combined, dried over anhydrous Na₂SO₄ and concentrated to give the crude product, which was purified by silica gel column chromatography (eluting with 0-10% MeOH in DCM) to give (S)-2-((tert-butoxycarbonyl)amino)-3-(3-(2-(5-(dimethylphosphorothioyl)-2-((S)-1-methoxyethyl)phenyl)-1-ethyl-3-(3-hydroxy-2,2-dimethylpropyl)-1H-indol-5-yl)-5-((triisopropylsilyl)oxy)phenyl)propanoic acid (100 mg, 62.2%) as a brown solid.

LCMS (ESI) calcd. for C₄₉H₇₃N₂O₇PSSi [M+H]⁺ m/z 893.46, found: 893.8

### Step 7. Synthesis of methyl (S)-1-((S)-2-((tert-butoxycarbonyl)amino)-3-(3-(2-(5-(dimethylphosphorothioyl)-2-((S)-1-methoxyethyl)phenyl)-1-ethyl-3-(3-hydroxy-2,2-dimethylpropyl)-1H-indol-5-yl)-5-((triisopropylsilyl)oxy)phenyl)propanoyl)hexahydropyridazine-3-carboxylate

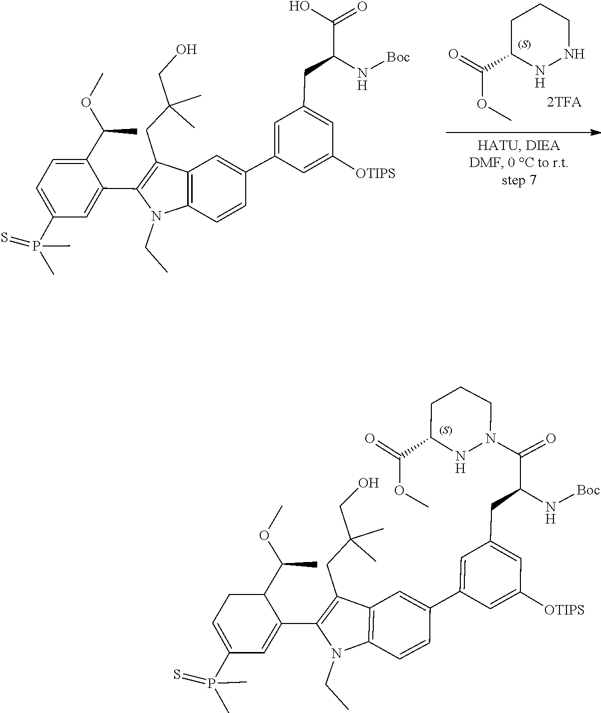

To a solution of (S)-2-((tert-butoxycarbonyl)amino)-3-(3-(2-(5-(dimethylphosphorothioyl)-2-((S)-1-methoxyethyl)phenyl)-1-ethyl-3-(3-hydroxy-2,2-dimethylpropyl)-1H-indol-5-yl)-5-((triisopropylsilyl)oxy)phenyl)propanoic acid (100 mg, 0.11 mmol) in DMF (1.5 mL) were added HATU (64 mg, 0.17 mmol) and DIEA (65 mg, 0.50 mmol) at 0-10° C. The resulting mixture was stirred at the same temperature for 30 min. Then a solution of methyl (S)-hexahydropyridazine-3-carboxylate (63 mg, 0.17 mmol) in DMF (1 mL) was added into the above mixture and the resulting mixture was stirred at rt for 20 h. The reaction was monitored by LCMS. After completion, the reaction mixture was diluted with brine (15 mL) and extracted with EtOAc (2×15 mL). The organic layers were combined, dried over anhydrous Na₂SO₄ and concentrated to give the crude product, which was purified by silica gel column chromatography (eluting with 0-5% methanol in DCM) to give methyl (S)-1-((S)-2-((tert-butoxycarbonyl)amino)-3-(3-(2-(5-(dimethylphosphorothioyl)-2-((S)-1-methoxyethyl)phenyl)-1-ethyl-3-(3-hydroxy-2,2-dimethylpropyl)-1H-indol-5-yl)-5-((triisopropylsilyl)oxy)phenyl)propanoyl)hexahydropyridazine-3-carboxylate (104 mg, 92.8%) as a yellow solid.

LCMS (ESI) calcd. for C₅₅H₈₃N₄O₈PSSi [M+H]⁺ m/z 1019.54, found: 1019.7

### Step 8. Synthesis of(S)-1-((S)-2-((tert-butoxycarbonyl)amino)-3-(3-(2-(5-(dimethylphosphorothioyl)-2-((S)-1-methoxyethyl)phenyl)-1-ethyl-3-(3-hydroxy-2,2-dimethylpropyl)-1H-indol-5-yl)-5-((triisopropylsilyl)oxy)phenyl)propanoyl)hexahydropyridazine-3-carboxylic acid

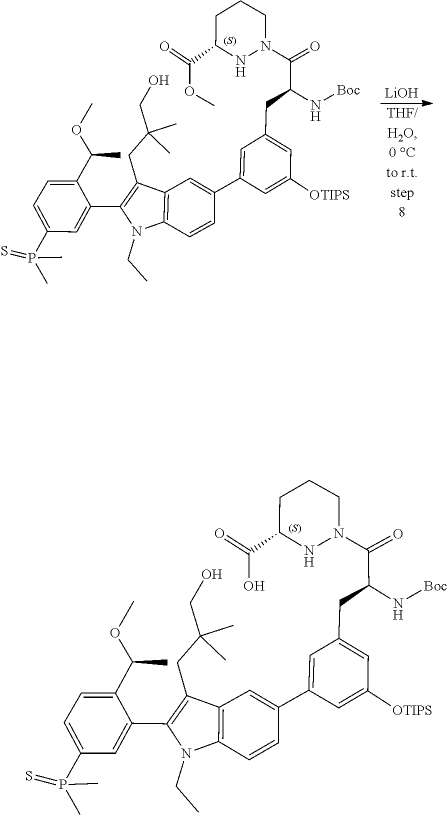

To a solution of methyl (S)-1-((S)-2-((tert-butoxycarbonyl)amino)-3-(3-(2-(5-(dimethylphosphorothioyl)-2-((S)-1-methoxyethyl)phenyl)-1-ethyl-3-(3-hydroxy-2,2-dimethylpropyl)-1H-indol-5-yl)-5-((triisopropylsilyl)oxy)phenyl)propanoyl)hexahydropyridazine-3-carboxylate (104 mg, 0.10 mmol) in THF (1.5 mL) and H₂O (0.75 mL) was added LiOH (13 mg, 0.31 mmol) at 0-10° C. and the resulting mixture was stirred at the same temperature for 1 h. The reaction was monitored by LCMS. After completion, the reaction mixture was diluted with H₂O (20 mL) and acidified to pH=4-5 with 2 N HCl. The resulting mixture was extracted with DCM (2×10 mL). The organic layers were combined, dried over anhydrous Na₂SO₄ and concentrated to give the crude (S)-1-((S)-2-((tert-butoxycarbonyl)amino)-3-(3-(2-(5-(dimethylphosphorothioyl)-2-((S)-1-methoxyethyl)phenyl)-1-ethyl-3-(3-hydroxy-2,2-dimethylpropyl)-1H-indol-5-yl)-5-((triisopropylsilyl)oxy)phenyl)propanoyl)hexahydropyridazine-3-carboxylic acid (85 mg, 84.5%) as a yellow solid, which was used for the next step directly.

LCMS (ESI) calcd. for C₅₄H₈₁N₄O₈PSSi [M+H]⁺ m/z 1005.53, found: 1005.9

### Step 9. Synthesis of tert-butyl ((6³S,4S)-1²-(5-(dimethylphosphorothioyl)-2-((S)-1-methoxyethyl)phenyl)-1¹-ethyl-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate

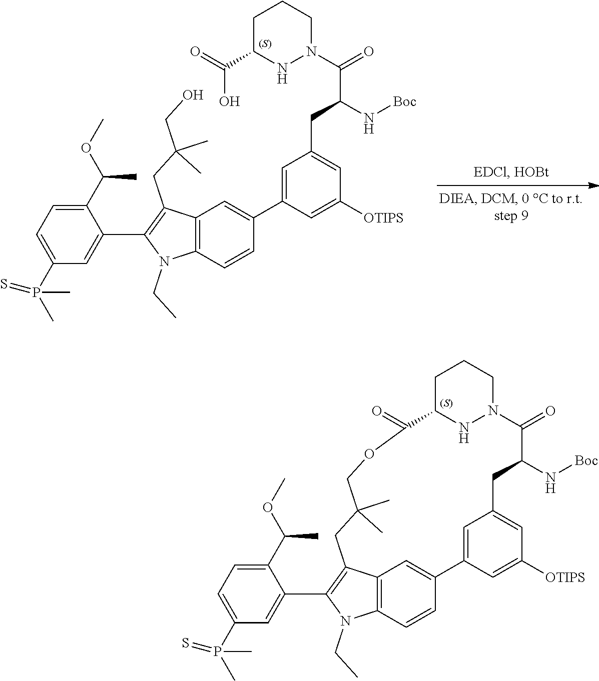

To a solution of crude (S)-1-((S)-2-((tert-butoxycarbonyl)amino)-3-(3-(2-(5-(dimethylphosphorothioyl)-2-((S)-1-methoxyethyl)phenyl)-1-ethyl-3-(3-hydroxy-2,2-dimethylpropyl)-1H-indol-5-yl)-5-((triisopropylsilyl)oxy)phenyl)propanoyl)hexahydropyridazine-3-carboxylic acid (85 mg, 0.085 mmol) and DIEA (33 mg, 0.25 mmol) in dry DCM (15 mL) under ice-water bath was added EDCI (49 mg, 0.25 mmol) and HOBt (23 mg, 0.17 mmol) by one portion. The reaction was gradually up to room temperature and stirred for 20 h and the reaction was monitored by LCMS. After completion, the reaction mixture was washed with H₂O (20 mL×3), then dried over with Na₂SO₄, following with concentration under reduced pressure to obtain crude product, which was purified by silica gel column (eluting with MeOH/DCM from 0% to 50%) to give tert-butyl ((6³S,4S)-1²-(5-(dimethylphosphorothioyl)-2-((S)-1-methoxyethyl)phenyl)-1¹-ethyl-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (38 mg, 45.6%) as a yellow gum.

LCMS (ESI) calcd. for C₅₄H₇₉N₄O₇PSSi [M+H]⁺ m/z 987.52, found: 987.9

### Step 10. Synthesis of (6³S,4S)-4-amino-1²-(5-(dimethylphosphorothioyl)-2-((S)-1-methoxyethyl)phenyl)-1¹-ethyl-10,10-dimethyl-25-((triisopropylsilyl)oxy)-6¹,6²,6³,6⁴,6⁵,6⁶-hexahydro-1′H-8-oxa-1(5,3)-indola-6(1,3)-pyridazina-2(1,3)-benzenacycloundecaphane-5,7-dione

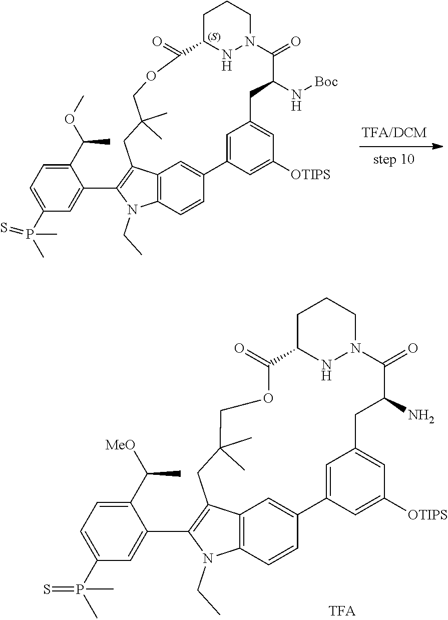

To a stirred solution of tert-butyl ((6³S,4S)-1²-(5-(dimethylphosphorothioyl)-2-((S)-1-methoxyethyl)phenyl)-1¹-ethyl-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (685 mg, 0.76 mmol) in dichloromethane (1.2 mL) was added TFA (0.1 mL) dropwisely at rt and the resulting mixture was stirred at rt for 1 h. The reaction mixture was concentrated to give the crude product, which was purified by pre-TLC (DCM:MeOH=12:1) to afford (6³S,4S)-4-amino-1²-(5-(dimethylphosphorothioyl)-2-((S)-1-methoxyethyl)phenyl)-1¹-ethyl-10,10-dimethyl-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione 12 (26 mg, 81.2%) as a white solid of its TFA salt.

LCMS (ESI) calcd. for C₄₉H₇₁BN₄O₅PSSi [M+H]⁺ m/z 887.47, found: 887.7

### Step 11. Synthesis of (2R,3R)—N-((2S)-1-(((6³S,4S)-1²-(5-(dimethylphosphorothioyl)-2-((S)-1-methoxyethyl)phenyl)-1¹-ethyl-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-N-methyl-3-phenyltetrahydrofuran-2-carboxamide

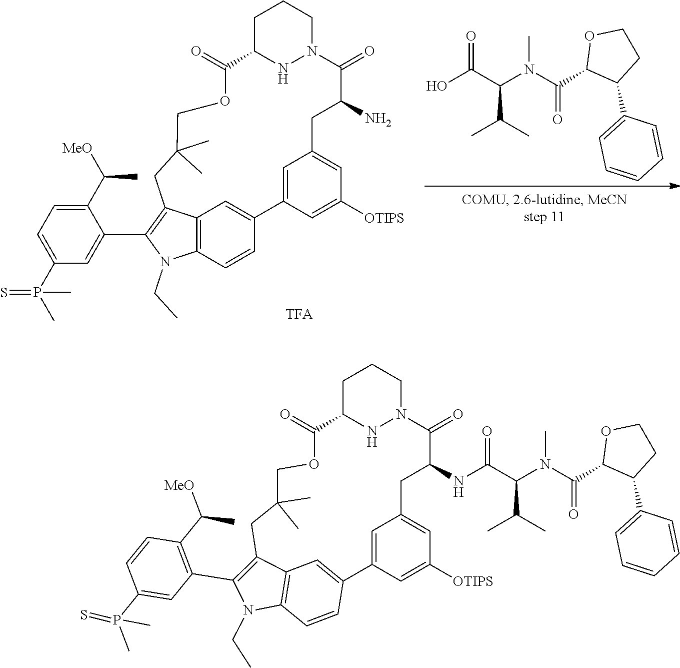

To a stirred solution of (6³S,4S)-4-amino-1²-(5-(dimethylphosphorothioyl)-2-((S)-1-methoxyethyl)phenyl)-11-ethyl-10,10-dimethyl-25-((triisopropylsilyl)oxy)-6¹,6²,6³,6⁴,6⁵,6⁶-hexahydro-11H-8-oxa-1(5,3)-indola-6(1,3)-pyridazina-2(1,3)-benzenacycloundecaphane-5,7-dione (26 mg, 0.026 mmol), N-methyl-N-((2R,3R)-3-phenyltetrahydrofuran-2-carbonyl)-L-valine (12 mg, 0.039 mmol) and 2,6-lutidine (14 mg, 0.13 mmol) in MeCN (2 mL) was added COMU (17 mg, 0.13 mmol) at rt and the resulting mixture was stirred at rt for 17 h. The reaction mixture was concentrated to give the crude product, which was purified by pre-TLC (DCM:MeOH=18:1) to give (2R,3R)—N-((2S)-1-(((6³S,4S)-1²-(5-(dimethylphosphorothioyl)-2-((S)-1-methoxyethyl)phenyl)-1¹-ethyl-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-N-methyl-3-phenyltetrahydrofuran-2-carboxamide (24 mg, 78.6%) as a white solid.

LCMS (ESI) calcd. for C₆₆H₉₂N₅O₈PSSi [M+H]⁺ m/z 1174.62, found: 1174.9

### Step 12. Synthesis of (2R,3R)—N-((2S)-1-(((6³S,4S)-1²-(5-(dimethylphosphorothioyl)-2-((S)-1-methoxyethyl)phenyl)-1¹-ethyl-2⁵-hydroxy-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-N-methyl-3-phenyltetrahydrofuran-2-carboxamide

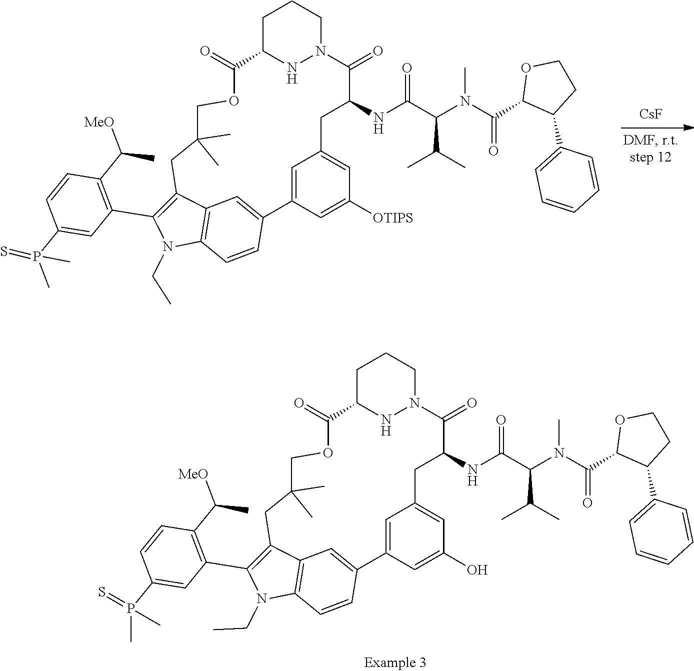

To a stirred solution of (2R,3R)—N-((2S)-1-(((6³S,4S)-1²-(5-(dimethylphosphorothioyl)-2-((S)-1-methoxyethyl)phenyl)-1¹-ethyl-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-N-methyl-3-phenyltetrahydrofuran-2-carboxamide (24 mg, 0.020 mmol) in DMF (2 mL) was added CsF (16 mg, 0.10 mmol) at rt and the resulting mixture was stirred at the same temperature for 2 h. The reaction mixture was diluted with H₂O and extracted with EtOAc (2×15 mL). The organic layers were combined, dried over anhydrous Na₂SO₄ and concentrated to give the crude product. The crude product was then purified by pre-TLC (DCM: MeOH=20:1) to give (2R,3R)—N-((2S)-1-(((6³S,4S)-1²-(5-(dimethylphosphorothioyl)-2-((S)-1-methoxyethyl)phenyl)-1¹-ethyl-2⁵-hydroxy-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-N-methyl-3-phenyltetrahydrofuran-2-carboxamide (5 mg, 25%) as a white solid as a white solid

LCMS (ESI) calcd. for C₅₇H₇₂N₅O₈PS [M+H]⁺ m/z 1018.48, found: 1018.9

### Biological Activity Results

### Ras: RAF1 HTRF Assay

This example is to verify the binding between RAS protein and its interacting protein (SOS1 or RAF1). When RAS exists with SOS1 or RAF, the two interact to make the energy donor and receptor bound to it close enough, and then when the donor is excited by an external light source, energy resonance transfer occurs, thereby exciting the receptor and emitting a specific wavelength of emitted light, the FRET signal is detected.

Specific experimental methods: 20 nM purified RAS protein (e.g. KRAS-G12D, G12V, G12C, WT; NRAS-WT) was incubated with the drug to be screened (the drug was diluted in a 3-fold gradient to a final concentration of 1000 nM) at room temperature for 1 h. Subsequently, 30 nM purified SOS1-Cat was added and incubated at room temperature for 0.5-1 h. After adding the detection antibody (PerkinElmer, Cisbio, HTRF antibody), the value was measured according to the manufacturer's instructions.

### The Representative Compound Results

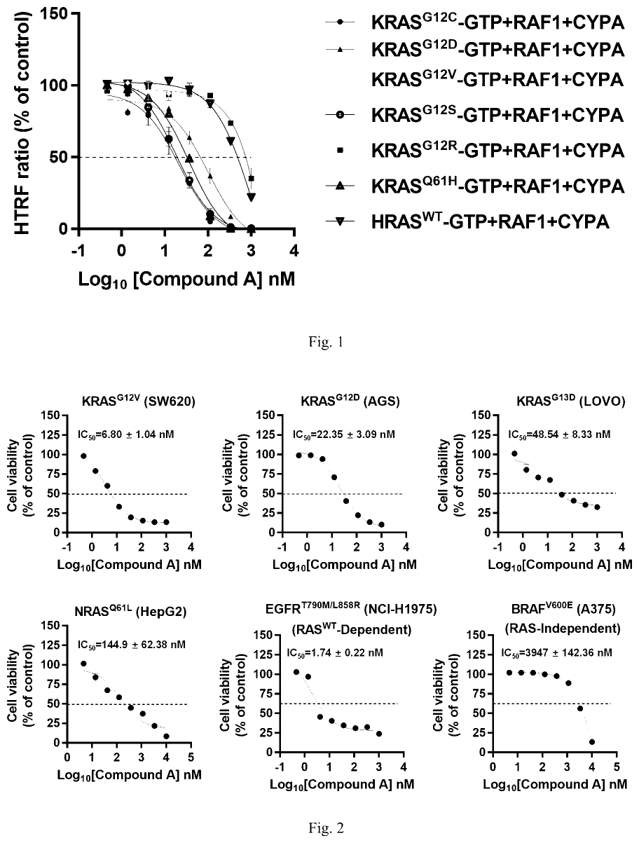

The representative Example 2 significantly inhibited the interaction of RAF1 with active KRAS mutant protein or HRAS WT protein, and the result is shown in FIG. 1. The inhibitory IC50 of the compounds of the examples of the present invention was range from 15.79 nM to 729 nM.

| RAS-GTP & Raf Disruption/HTRF Assay | IC50(nM) |
|---|---|
| KRAS^{G12C}GTP + RAF1 + CYPA | 15.79 ± 3.97 |
| KRAS^{G12D}GTP + RAF1 + CYPA | 55.92 ± 8.92 |
| KRAS^{G12V}GTP + RAF1 + CYPA | 164.7 ± 7.92 |
| KRAS^{G12S}GTP + RAF1 + CYPA | 19.61 ± 1.03 |
| KRAS^{G12R}GTP + RAF1 + CYPA | 729.4 ± 105.57 |
| KRAS^{Q61H}GTP + RAF1 + CYPA | 31.24 ± 0.30 |
| HRAS^{WT}GTP + RAF1 + CYPA | 467.8 ± 4.60 |

### Cell Viability Assay

Selective screening using RAS-driven and RAS-non-driven cells: (1) RAS-driven cells, including cells with persistent RAS activation due to RAS mutations and upstream EGFR activation. Cells that are continuously activated due to RAS mutations, such as KRAS G12D (AsPC-1, HPAC, etc.) mutant cells, KRAS G12C (NCI-H358, MIAPaCa-2, etc.) KRAS G13D (LOVO, HCT15, etc.) mutant cells, etc. Cells with continuous activation of RAS due to EGFR mutations, such as EGFREx19del (PC-9, etc.) mutant cells, EGFRT790M/L858R (NCI-H1975, etc.) mutant cells, etc.; (2) Non-RAS driven cells, including BRAFV600E (A375, SK)-MEL-3, etc.) mutant cells, RASWT (BxPC-3, etc.) cells, etc.

Specific experimental methods: The above cells were seeded in a 96-well plate at a density of 1000-10000 per well. Drugs were diluted in DMSO to make a 10 mM stock solution. Subsequently, the drug was diluted in a 3-fold gradient to a final concentration of 1000 nM. The cells were treated with the drug for 5 days. Cell viability was then detected by the MTS method.

### The Representative Compound Results

The representative Example 2 significantly inhibited the cell viability of KRAS mutant cells: SW620 (KRAS G12V), AGS (KRAS G12D), LOVO (KRAS G13D); NRAS mutant cells: HepG2 (NRAS Q61L); EGFR mutant cells: NCI-H1975 (EGFR T790M/L858R). On the contrast, Example 2 was relative insensitive RAS-independent cells: A375 (BRAF V600E). and the esult is shown in FIG. 2.

1. A compound of formula (I), or a pharmaceutically acceptable salt thereof, a solvate thereof or a solvate of the pharmaceutically acceptable salt, 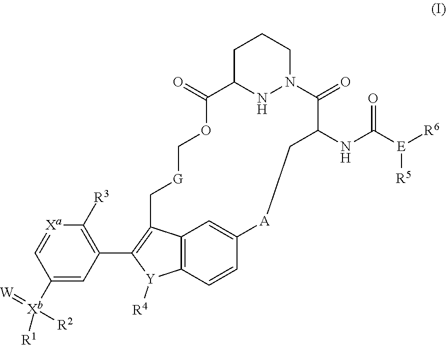
Wherein, A is 5-6 membered heterocycloalkylene, 5-6 membered heteroarylene, phenylene or phenylene optionally substituted by hydroxy or boric acid group;
G is C₁₋₆alkylene;
X^{a} is CH or N;
X^{b} is P or B;
Y is O, S or N;
W is absent, O or S, provided that when X^{b} is B, W is absent;
R¹ and R² are each independently selected from hydroxy, C₁₋₆ alkyl, C₁₋₆ alkoxyl, C₁₋₆ haloalkyl, C₁₋₆ haloalkoxyl, or R¹ and R² combine with the atoms to which they are attached to form a 4 to 10-membered ring, wherein the ring is selected from cycloalkyl optionally substituted by halogen, C₁₋₆ alkyl, C₁₋₆ haloalkyl, C₁₋₆ haloalkoxy; heterocycloalkyl optionally substituted by halogen, C₁₋₆ alkyl, C₁₋₆haloalkyl, C₁₋₆ haloalkoxy;
R³ is C₁₋₆ alkoxyC₁₋₆ alkyl;
R⁴ is absent, or selected from hydrogen, C₁₋₆ alkyl, or C₁₋₆ haloalkyl, provided that when Y is O or S, R⁴ is absent;
E is a bond, or selected from N, or —CR^{A}—, wherein R^{A} is selected from halogen, hydroxy, C₁₋₆ alkyl, C₁₋₆ haloalkyl, C₁₋₆ alkoxyl, C₁₋₆ AlkoxyC₁₋₆ alkyl, —NR^{B}R^{C}, cyano, C₁₋₆ aminoalkyl, C₁₋₆ hydroxyalkyl, or carboxyl; wherein R^{B} and R^{C} are independently selected from hydrogen, or C₁₋₆ alkyl;
R⁵ is hydrogen, C₁₋₆ alkyl, C₁₋₆ heteroalkyl, C₃₋₁₀ heterocycloalkyl, C₃₋₁₀ cycloalkyl, C₆₋₁₀ aryl, C₅₋₁₀ heteroaryl, or 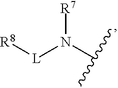
wherein R⁷ is hydrogen, or C₁₋₆ alkyl;
R¹ is C₁₋₆ alkyl, C₁₋₆ heteroalkyl, C₃₋₁₀heterocycloalkyl, C₃₋₁₀ cycloalkyl, C₆₋₁₀ aryl, C₅₋₁₀ heteroaryl;
L is absent, or selected from —CH₂—, —C(O)—, —CHR⁸— or —C(R⁸)₂—, wherein R⁸ is C₁₋₆alkyl;
R⁶ is hydrogen, C₁₋₆ alkyl or C₃₋₆ cycloalkyl;
or R⁵ and R⁶ combine with the atoms to which they are attached to form a ring, wherein the ring is selected from 3- to 10-membered cycloalkyl optionally substituted by at least one of halogen, C₁₋₆ alkyl, C₁₋₆ haloalkyl, C₁₋₆ alkoxyl, C₁₋₆ haloalkoxyl, C₁₋₆ hydroxyalkyl, C₁₋₆ alkoxyC₁₋₆alkyl, or oxo; 3- to 10-membered heterocycloalkyl optionally substituted by at least one of halogen, hydroxyl, C₁₋₆ alkyl, C₁₋₆ alkoxyl, C₁₋₆ haloalkyl, C₁₋₆ haloalkoxyl, C₁₋₆ alkoxyC₁₋₆ alkyl, C₁₋₆ haloalkoxyC₁₋₆ alkyl, C₆₋₁₀ aryl, C₅₋₁₀ heteroaryl, C(O)C₁₋₆ alkyl, C(O)OC₁₋₆ alky, or oxo; or R^{A}, R⁵ and R⁶ combine with the atoms to which they are attached to form a bicyclo C₇₋₁₄ heterocycloalkyl.

2. the compound of formula (I), or the pharmaceutically acceptable salt thereof, the solvate thereof or the solvate of the pharmaceutically acceptable salt according to claim 1, wherein, the compound of formula (I) is a compound of formula (Ia) or (Ib), 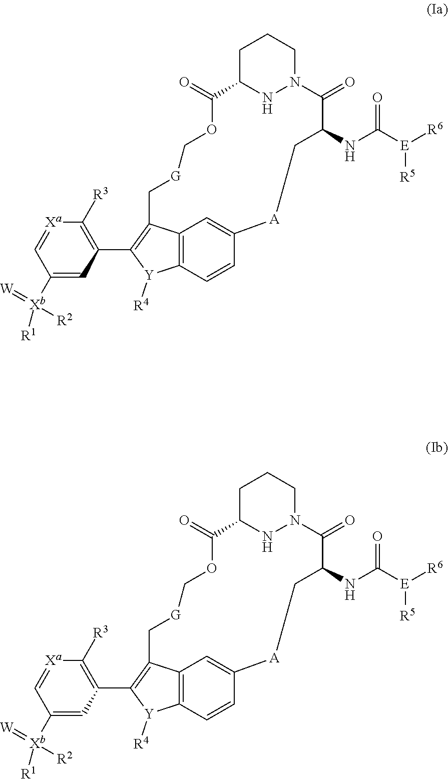
Wherein, A is 5-6 membered heterocycloalkylene, 5-6 membered heteroarylene, phenylene or phenylene optionally substituted by hydroxy or boric acid group;
G is C₁₋₆alkylene;
X^{a} is CH or N;
X^{b} is P or B;
Y is O, S or N;
W is absent, O or S, provided that when X^{b} is B, W is absent;
R¹ and R² are each independently selected from hydroxy, C₁₋₆ alkyl, C₁₋₆ alkoxyl, C₁₋₆ haloalkyl, C₁₋₆ haloalkoxyl, or R¹ and R² combine with the atoms to which they are attached to form a 4 to 10-membered ring, wherein the ring is selected from cycloalkyl optionally substituted by halogen, C₁₋₆ alkyl, C₁₋₆ haloalkyl, C₁₋₆ haloalkoxy; heterocycloalkyl optionally substituted by halogen, C₁₋₆ alkyl, C₁₋₆ haloalkyl, C₁₋₆ haloalkoxy;
R³ is C₁₋₆ alkoxyC₁₋₆ alkyl;
R⁴ is absent, or selected from hydrogen, C₁₋₆ alkyl, or C₁₋₆ haloalkyl, provided that when Y is O or S, R⁴ is absent;
E is a bond, or selected from N, or —CR^{A}—, wherein R^{A} is selected from halogen, hydroxy, C₁₋₆ alkyl, C₁₋₆ haloalkyl, C₁₋₆ alkoxyl, C₁₋₆ AlkoxyC₁₋₆ alkyl, —NR^{B}R^{C}, cyano, C₁₋₆ aminoalkyl, C₁₋₆ hydroxyalkyl, or carboxyl; wherein R^{B} and R^{C} are independently selected from hydrogen, or C₁₋₆ alkyl;
R⁵ is hydrogen, C₁₋₆ alkyl, C₁₋₆ heteroalkyl, C₃₋₁₀ heterocycloalkyl, C₃₋₁₀ cycloalkyl, C₆₋₁₀ aryl, C₅₋₁₀ heteroaryl, or 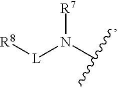
wherein R⁷ is hydrogen, or C₁₋₆ alkyl;
R⁸ is C₁₋₆ alkyl, C₁₋₆ heteroalkyl, C₃₋₁₀ heterocycloalkyl, C₃₋₁₀ cycloalkyl, C₆₋₁₀ aryl, C₅₋₁₀ heteroaryl;
L is absent, or selected from —CH₂—, —C(O)—, —CHR⁸— or —C(R⁸)₂—, wherein R⁸ is C₁₋₆ alkyl;
R⁶ is hydrogen, C₁₋₆alkyl or C₃₋₆cycloalkyl;
or R⁵ and R⁶ combine with the atoms to which they are attached to form a ring, wherein the ring is selected from 3- to 10-membered cycloalkyl optionally substituted by at least one of halogen, C₁₋₆ alkyl, C₁₋₆ haloalkyl, C₁₋₆ alkoxyl, C₁₋₆ haloalkoxyl, C₁₋₆ hydroxyalkyl, C₁₋₆ alkoxyC₁₋₆alkyl, or oxo; 3- to 10-membered heterocycloalkyl optionally substituted by at least one of halogen, hydroxyl, C₁₋₆alkyl, C₁₋₆alkoxyl, C₁₋₆ haloalkyl, C₁₋₆haloalkoxyl, C₁₋₆ alkoxyC₁₋₆alkyl, C₁₋₆ haloalkoxyC₁₋₆alkyl, C₆₋₁₀ aryl, C₅₋₁₀ heteroaryl, C(O)C₁₋₆ alkyl, C(O)OC₁₋₆ alky, or oxo; or R^{A}, R⁵ and R⁶ combine with the atoms to which they are attached to form a bicyclo C₇₋₁₄ heterocycloalkyl.

3. the compound of formula (I), or the pharmaceutically acceptable salt thereof, the solvate thereof or the solvate of the pharmaceutically acceptable salt according to claim 1, wherein, 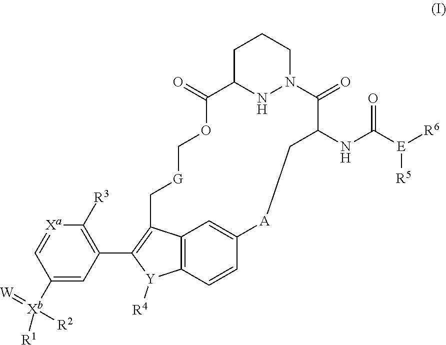
wherein
A is 5-6 membered heterocycloalkylene, 5-6 membered heteroarylene, phenylene, or the phenylene optionally substituted by hydroxy or boric acid group;
G is C₁₋₆ alkylene;
X^{a} is CH or N;
X^{b} is P or B;
Y is O, S or N;
W is absent, O or S, provided that when X^{b} is B, W is absent;
R¹ and R² are each independently selected from hydroxy, C₁₋₆ alkyl, C₁₋₆ alkoxyl, C₁₋₆ haloalkyl, C₁₋₆ haloalkoxyl, or R¹ and R² combine with the atoms to which they are attached to form a 4 to 10-membered ring, wherein the ring is heterocycloalkyl; and the heterocycloalkyl optionally substituted by halogen, C₁₋₆ alkyl, C₁₋₆ haloalkyl, C₁₋₆ haloalkoxy;
R³ is C₁₋₆ alkoxyC₁₋₆alkyl;
R⁴ is absent, or selected from hydrogen, C₁₋₆ alkyl, or C₁₋₆ haloalkyl, provided that when Y is O or S, R⁴ is absent;
E is a bond, or selected from N, or —CR^{A}—, wherein R^{A} is selected from hydrogen, halogen, hydroxy, C₁₋₆ alkyl, C₁₋₆ haloalkyl, C₁₋₆ alkoxyl, C₁₋₆alkyl substituted by C₁₋₆ alkoxy, —NR^{B}R^{C}, cyano, C₁₋₆ aminoalkyl, C₁₋₆ hydroxyalkyl, or carboxyl; wherein R^{B} and R^{C} are independently selected from hydrogen, or C₁₋₆ alkyl;
R⁵ is hydrogen, C₁₋₆ alkyl, C₁₋₆ heteroalkyl, C₃₋₁₀ heterocycloalkyl, C₃₋₁₀ cycloalkyl, C₆₋₁₀ aryl, C₅₋₁₀ heteroaryl, or 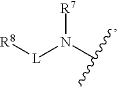
wherein R⁷ is hydrogen, or C₁₋₆ alkyl;
R⁸ is C₁₋₆ alkyl, C₁₋₆ heteroalkyl, C₃₋₁₀ heterocycloalkyl, C₃₋₁₀ cycloalkyl, C₆₋₁₀ aryl, C₅₋₁₀ heteroaryl; the C₃₋₁₀ heterocycloalkyl optionally substituted by C₁₋₆ alkyl, C₆₋₁₀ aryl, —CO—O—C₃₋₆ cycloalkyl or —CO—O—C₁₋₆ alkyl; the C₃₋₁₀ cycloalkyl and C₃₋₆ cycloalkyl optionally substituted by hydroxyl, cyano, halogen, or C₁₋₆ alkyl
L is absent, or selected from —CH₂—, —C(O)—, —CHR⁹— or —C(R⁹)₂—, wherein R⁹ is C₁₋₆ alkyl;
R⁶ is hydrogen, C₁₋₆ alkyl or C₃₋₆ cycloalkyl;
or R⁵ and R⁶ combine with the atoms to which they are attached to form a ring, wherein the ring is selected from 3- to 10-membered cycloalkyl and 3- to 10-membered heterocycloalkyl; the 3- to 10-membered cycloalkyl optionally substituted by at least one of halogen, C₁₋₆ alkyl, C₁₋₆ haloalkyl, C₁₋₆ alkoxyl, C₁₋₆ haloalkoxyl, C₁₋₆ hydroxyalkyl, C₁₋₆ alkoxyC₁₋₆ alkyl, or oxo; the 3- to 10-membered heterocycloalkyl optionally substituted by at least one of halogen, hydroxyl, C₁₋₆alkyl, C₁₋₆ alkoxyl, C₁₋₆ haloalkyl, C₁₋₆ haloalkoxyl, —C₁₋₆ alkoxyC₁₋₆ alkyl, C₁₋₆ haloalkoxyC₁₋₆ alkyl, C₆₋₁₀ aryl, C₅₋₁₀ heteroaryl, C(O)C₁₋₆ alkyl, C(O)OC₁₋₆ alky, or oxo; or R^{A}, R⁵ and R⁶ combine with the atoms to which they are attached to form a bicyclo C₇₋₁₄ heterocycloalkyl;
in the 5-6 membered heterocycloalkylene, 5-6 membered heteroarylene, heterocycloalkyl, C₁₋₆ heteroalkyl, C₃₋₁₀ heterocycloalkyl, C₅₋₁₀ heteroaryl, bicyclo C₇₋₁₄ heterocycloalkyl and 3- to 10-membered heterocycloalkyl, the heteroatom is independently selected from one or more of N, O and S, and the number of the heteroatom is independently 1-3.

4. the compound of formula (I), or the pharmaceutically acceptable salt thereof, the solvate thereof or the solvate of the pharmaceutically acceptable salt according to claim 3, wherein, the compound of formula (I) is a compound of formula (Ia) or (Ib), 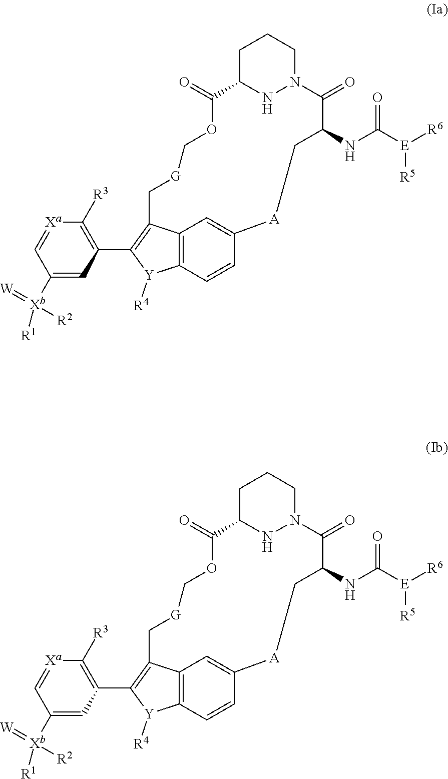
wherein
A is 5-6 membered heterocycloalkylene, 5-6 membered heteroarylene, phenylene, or the phenylene optionally substituted by hydroxy or boric acid group;
G is C₁₋₆ alkylene;
X^{a} is CH or N;
X^{b} is P or B;
Y is O, S or N;
W is absent, O or S, provided that when X^{b} is B, W is absent;
R¹ and R² are each independently selected from hydroxy, C₁₋₆ alkyl, C₁₋₆ alkoxyl, C₁₋₆ haloalkyl, C₁₋₆ haloalkoxyl, or R¹ and R² combine with the atoms to which they are attached to form a 4 to 10-membered ring, wherein the ring is heterocycloalkyl; and the heterocycloalkyl optionally substituted by halogen, C₁₋₆ alkyl, C₁₋₆haloalkyl, C₁₋₆ haloalkoxy;
R³ is or C₁₋₆ alkyl substituted by C₁₋₆alkoxy;
R⁴ is absent, or selected from hydrogen, C₁₋₆ alkyl, or C₁₋₆ haloalkyl, provided that when Y is O or S, R⁴ is absent;
E is a bond, or selected from N, or —CR^{A}—, wherein R^{A} is selected from hydrogen, halogen, hydroxy, C₁₋₆ alkyl, C₁₋₆ haloalkyl, C₁₋₆ alkoxyl, C₁₋₆ alkyl substituted by C₁₋₆ alkoxy, —NR^{B}R^{C}, cyano, C₁₋₆ aminoalkyl, C₁₋₆ hydroxyalkyl, or carboxyl; wherein R^{B} and R^{C} are independently selected from hydrogen, or C₁₋₆ alkyl;
R⁵ is hydrogen, C₁₋₆ alkyl, C₁₋₆ heteroalkyl, C₃₋₁₀ heterocycloalkyl, C₃₋₁₀ cycloalkyl, C₆₋₁₀ aryl, C₅₋₁₀ heteroaryl, or 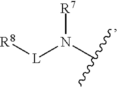
wherein R⁷ is hydrogen, or C₁₋₆ alkyl;
R⁸ is C₁₋₆ alkyl, C₁₋₆ heteroalkyl, C₃₋₁₀ heterocycloalkyl, C₃₋₁₀ cycloalkyl, C₆₋₁₀ aryl, C₅₋₁₀ heteroaryl; the C₃₋₁₀heterocycloalkyl optionally substituted by C₁₋₆ alkyl, C₆₋₁₀ aryl, —CO—O—C₃₋₆ cycloalkyl or —CO—O—C₁₋₆ alkyl; the C₃₋₁₀ cycloalkyl and C₃₋₆ cycloalkyl optionally substituted by hydroxyl, cyano, halogen, or C₁₋₆ alkyl;
L is absent, or selected from —CH₂—, —C(O)—, —CHR⁹— or —C(R⁹)₂—, wherein R⁹is C₁₋₆ alkyl;
R⁶ is hydrogen, C₁₋₆ alkyl or C₃₋₆ cycloalkyl;
or R⁵ and R⁶ combine with the atoms to which they are attached to form a ring, wherein the ring is selected from 3- to 10-membered cycloalkyl and 3- to 10-membered heterocycloalkyl; the 3- to 10-membered cycloalkyl optionally substituted by at least one of halogen, C₁₋₆ alkyl, C₁₋₆ haloalkyl, C₁₋₆ alkoxyl, C₁₋₆ haloalkoxyl, C₁₋₆ hydroxyalkyl, C₁₋₆ alkoxyC₁₋₆ alkyl, or oxo; the 3- to 10-membered heterocycloalkyl optionally substituted by at least one of halogen, hydroxyl, C₁₋₆ alkyl, C₁₋₆ alkoxyl, C₁₋₆ haloalkyl, C₁₋₆ haloalkoxyl, C₁₋₆ alkoxyC₁₋₆ alkyl, C₁₋₆ haloalkoxyC₁₋₆ alkyl, C₆₋₁₀ aryl, C₅₋₁₀ heteroaryl, C(O)C₁₋₆ alkyl, C(O)OC₁₋₆ alky, or oxo;
or R^{A}, R⁵ and R⁶ combine with the atoms to which they are attached to form a bicyclo C₇₋₁₄ heterocycloalkyl;
in the 5-6 membered heterocycloalkylene, 5-6 membered heteroarylene, heterocycloalkyl, C₁₋₆ heteroalkyl, C₃₋₁₀ heterocycloalkyl, C₅₋₁₀ heteroaryl, bicyclo C₇₋₁₄ heterocycloalkyl, and 3- to 10-membered heterocycloalkyl, the heteroatom is independently selected from one or more of N, O and S, and the number of the heteroatom is independently 1-3.

5. the compound of formula (I), or the pharmaceutically acceptable salt thereof, the solvate thereof or the solvate of the pharmaceutically acceptable salt according to claim 1, wherein, the compound of formula (I) meets one or more of the following conditions:
(1) in A, in the 5-6 membered heterocycloalkylene, the heteroatom is independently selected from one or two of N, O and S, and the number of the heteroatom is independently 1-2;
(2) in A, in 5-6 membered heteroarylene, the heteroatom is independently selected from one or two of N, O and S, and the number of the heteroatom is independently 1-2, such as 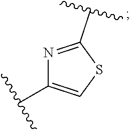 [SMILES: *c1csc(*)n1]
(3) in R¹ and R², the C₁₋₆ alkyl is methyl, ethyl, propyl or isopropyl;
(4) in R¹ and R², the C₁₋₆ alkoxyl is methoxy, ethoxy, n-propoxy or isopropoxy;
(5) in R¹ and R², the C₁₋₆ haloalkyl is —CF₃;
(6) in R¹ and R², the C₁₋₆ haloalkoxyl is —O—CF₃;
(7) in R¹ and R², the 5 to 6-membered ring is heterocycloalkyl, the heteroatom is independently selected from one or two of N and P, and the number of the heteroatom is independently 1-2, preferably, 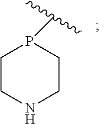 [SMILES: *P1CCNCC1]
(8) in R³, the C₁₋₆ alkoxyC₁₋₆alkyl is 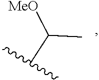 [SMILES: *C(C)OC]
preferably, 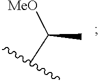 [SMILES: *[C@H](C)OC]
(9) in R⁴, the C₁₋₆ alkyl is methyl, ethyl, propyl or isopropyl;
(10) in R⁴, the C₁₋₆haloalky is —CF₃ or —CH₂CF₃;
(11) in R^{A}, the halogen is F, Cl, Br or I; preferably, F;
(12) in R^{A}, the C₁₋₆ alkyl is methyl, ethyl, propyl or isopropyl;
(13) in R^{A}, the C₁₋₆ haloalkyl is —CF₃;
(14) in R^{A}, the C₁₋₆ alkoxyl is -o-methyl, -o-ethyl, -o-propyl or -o-isopropyl;
(15) in R^{A}, the C₁₋₆alkyl is methyl, ethyl, propyl or isopropyl;
(16) in R^{A}, the C₁₋₆ aminoalkyl is —NH-methyl, —NH-ethyl, —NH-propyl or —NH— isopropyl;
(17) in R^{A}, the C₁₋₆ hydroxyalkyl is ydroxy-substituted methyl, hydroxy-substituted ethyl, hydroxy-substituted n-propyl or hydroxy-substituted isopropyl;
(18) in R^{B} and R^{C}, the C₁₋₆ alkyl is methyl, ethyl, propyl or isopropyl;
(19) in R⁵, the C₁₋₆ alkyl is methyl, ethyl, propyl or isopropyl;
(20) in R⁵, in C₁₋₆ heteroalkyl, the heteroatom is N, O or S, and the number of the heteroatom is independently 1-2;
(21) in R⁵, the 3- to 10-membered heterocycloalkyl is 3- to 7-membered heterocycloalkyl, preferably, the heteroatom is N, O or S, and the number of the heteroatom is independently 1-2;
(22) in R⁵, the C₃₋₁₀ cycloalkyl is C₃₋₆ cycloalkyl, preferably, cyclopropyl, cyclobutyl, cyclopentyl or cyclohexyl;
(23) in R⁵, the C₆₋₁₀ aryl is 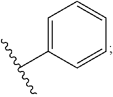 [SMILES: *c1ccccc1]
(24) in R⁸, the C₁₋₆ alkyl is methyl, ethyl, propyl or isopropyl;
(25) in R⁸, in C₁₋₆ heteroalkyl, the heteroatom is N, O or S, and the number of the heteroatom is independently 1-2;
(26) in R⁸, the C₃₋₁₀ heterocycloalkyl is 3- to 6-membered heterocycloalkyl, preferably, the heteroatom is N, O or S, and the number of the heteroatom is independently 1-2, preferably 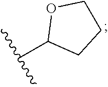 [SMILES: *C1CCCO1]
(27) in R⁸, the C₃₋₁₀ cycloalky is C₃₋₆ cycloalkyl, preferably, cyclopropyl, cyclobutyl, cyclopentyl or cyclohexyl;
(28) in R⁸, the C₆₋₁₀ aryl is 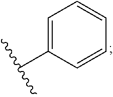 [SMILES: *c1ccccc1]
(29) in R⁸, the —CO—O—C₁₋₆ alkyl is —CO—O-methyl, —CO—O-ethyl, —CO—O-propyl or —CO—O-isopropyl;
(30) in R⁶, C₁₋₆ alkyl is methyl, ethyl, propyl or isopropyl;
(31) in R⁶, C₃₋₆ cycloalkyl is cyclopropyl, cyclobutyl, cyclopentyl or cyclohexyl;
(32) in G, C₁₋₆ alkylene is methylene, ethylene, propylene or isopropylidene;
(33) in bicyclo C₇₋₁₄ heterocycloalkyl, the heteroatom is N, O or S, and the number of the heteroatom is independently 1-2, preferably,

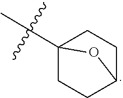 [SMILES: *C12CCC(CC1)O2]

6. the compound of formula (I), or the pharmaceutically acceptable salt thereof, the solvate thereof or the solvate of the pharmaceutically acceptable salt according to any one of claims 1-5, wherein, the compound of formula (I) meets one or more of the following conditions:
(1) A is 5-6 membered heterocycloalkylene, phenylene or phenylene optionally substituted by hydroxy or boric acid group; in the 5-6 membered heterocycloalkylene, the heteroatom is independently selected from one or more of N, O and S, and the number of the heteroatom is independently 1-3;
(2) G is C₁₋₆ alkylene;
(3) X^{a} is CH;
(4) R¹ and R² are each independently selected from hydroxy, C₁₋₆ alkyl, or R¹ and R² combine with the atoms to which they are attached to form a 4 to 10-membered ring, wherein the ring is heterocycloalkyl; the heterocycloalkyl optionally substituted by C₁₋₆ alkyl, in the 4 to 10-membered ring, the heteroatom is independently selected from one or more of N, O and S, and the number of the heteroatom is independently 1-3; preferably, R¹ and R² are each independently selected from hydroxy and C₁₋₆ alkyl,
(5) R³ is C₁₋₆ alkyl substituted by C₁₋₆ alkoxy;
(6) R⁴ is hydrogen, C₁₋₆ alkyl, or C₁₋₆ haloalkyl; preferably, R⁴ is hydrogen or C₁₋₆ alky;
(7) E is a bond, or selected from N, or —CR^{A}—, wherein R^{A} is selected from hydrogen, halogen, hydroxyC₁₋₆ alkyl, C₁₋₆ haloalkyl, C₁₋₆ alkoxyl, C₁₋₆ alkyl substituted by C₁₋₆ alkoxy, C₁₋₆ alkoxy substituted by C₁₋₆ alkyl, —NR^{B}R^{C}, cyano, C₁₋₆ aminoalkyl, C₁₋₆ hydroxyalkyl, or carboxyl; wherein R^{B} and R^{C} are independently selected from hydrogen, or C₁₋₆ alkyl; preferably, E is a bond, or selected from N, or —CR^{A}—, wherein R^{A} is selected from hydrogen, halogen, hydroxy or C₁₋₆ alkyl;
(8) R⁵ is hydrogen, C₁₋₆ alkyl, 3- to 10-membered heterocycloalkyl, C₃₋₁₀ cycloalkyl, C₆₋₁₀ aryl, C₅₋₁₀ heteroaryl, or 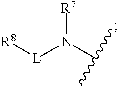
wherein R⁷ is hydrogen, or C₁₋₆ alkyl; R^{s} is C₁₋₆ alkyl, 3- to 10-membered heterocycloalkyl; the 3- to 10-membered heterocycloalkyl optionally substituted by C₁₋₆alkyl, C₆₋₁₀ aryl, —CO—O—C₃₋₆ cycloalkyl or —CO—O—C₁₋₆ alkyl; the C₃₋₁₀ cycloalkyl and C₃₋₆ cycloalkyl optionally substituted by hydroxyl, cyano, halogen, or C₁₋₆ alkyl; in 3- to 10-membered heterocycloalkyl, the heteroatom is independently selected from one or more of N, O and S, and the number of the heteroatom is independently 1-3; preferably, R⁵ is hydrogen, C₁₋₆ alkyl, 3- to 10-membered heterocycloalkyl, C₃₋₁₀ cycloalkyl, C₆₋₁₀ aryl, C₅₋₁₀ heteroaryl, or 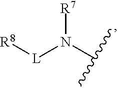
wherein R⁷ is hydrogen, or C₁₋₆ alkyl; R⁸ is C₁₋₆ alkyl, 3- to 10-membered heterocycloalkyl; the 3- to 10-membered heterocycloalkyl optionally substituted by C₁₋₆ alkyl, C₆₋₁₀ aryl, —CO—O—C₃₋₆ cycloalkyl or —CO—O—C₁₋₆ alkyl; the C₃₋₁₀ cycloalkyl and C₃₋₆ cycloalkyl optionally substituted by hydroxyl, cyano, halogen, or C₁₋₆ alkyl; in 3- to 10-membered heterocycloalkyl, the heteroatom is independently selected from one or more of N, O and S, and the number of the heteroatom is independently 1-3; more preferably, R⁵ is hydrogen, C₁₋₆ alkyl, or 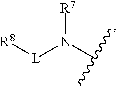
wherein R⁷ is hydrogen, or C₁₋₆ alkyl;
(9) R⁸ is C₁₋₆ alkyl, 3- to 6-membered heterocycloalkyl; the 3- to 6-membered heterocycloalkyl optionally substituted by C₆₋₁₀aryl or —CO—O—C₁₋₆alkyl; in 3- to 6-membered heterocycloalkyl, the heteroatom is independently selected from one or two of N and O, and the number of the heteroatom is independently 1-2.

7. the compound of formula (I), or the pharmaceutically acceptable salt thereof, the solvate thereof or the solvate of the pharmaceutically acceptable salt according to claim 1, wherein, the compound of formula (I) has the definition described in any of the following schemes:
scheme 1:
wherein A is 5-6 membered heterocycloalkylene, 5-6 membered heteroarylene, phenylene, or the phenylene optionally substituted by hydroxy or boric acid group;
G is C₁₋₆ alkylene;
X^{a} is CH or N;
X^{b} is P or B;
Y is N;
W is absent, O or S, provided that when X^{b} is B, W is absent;
R¹ and R² are each independently selected from hydroxy, C₁₋₆ alkyl, or R¹ and R² combine with the atoms to which they are attached to form a 4 to 10-membered ring, wherein the ring is heterocycloalkyl; the heterocycloalkyl optionally substituted by C₁₋₆ alkyl;
R³ is C₁₋₆ alkyl substituted by C₁₋₆ alkoxy;
R⁴ is hydrogen, C₁₋₆ alkyl, or C₁₋₆haloalkyl;
E is a bond, or selected from N, or —CR^{A}—, wherein R^{A} is selected from hydrogen, halogen, hydroxyC₁₋₆ alkyl, C₁₋₆ haloalkyl, C₁₋₆ alkoxyl, C₁₋₆ alkyl substituted by C₁₋₆ alkoxy, C₁₋₆ alkoxy substituted by C₁₋₆ alkyl, —NR^{B}R^{C}, cyano, C₁₋₆ aminoalkyl, C₁₋₆ hydroxyalkyl, or carboxyl; wherein R^{B} and R^{C} are independently selected from hydrogen, or C₁₋₆ alkyl:
R⁵ is hydrogen, C₁₋₆ alkyl, 3- to 10-membered heterocycloalkyl, C₃₋₁₀ cycloalkyl, C₆₋₁₀ aryl, C₅₋₁₀ heteroaryl, or 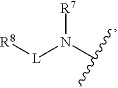
wherein R⁷ is hydrogen, or C₁₋₆ alkyl; R⁸ is C₁₋₆ alkyl, 3- to 10-membered heterocycloalkyl; the 3- to 10-membered heterocycloalkyl optionally substituted by C₁₋₆alkyl, C₆₋₁₀aryl, —CO—O—C₃₋₆ cycloalkyl or —CO—O—C₁₋₆ alkyl; the C₃₋₁₀ cycloalkyl and C₃₋₆ cycloalkyl optionally substituted by hydroxyl, cyano, halogen, or C₁₋₆ alkyl;
L is absent, or selected from —CH₂—, —C(O)—, —CHR⁹— or —C(R⁹)₂—, wherein R⁹ is C₁₋₆ alkyl;
R⁶ is hydrogen, C₁₋₆ alkyl or C₃₋₆ cycloalkyl;
or R⁵ and R⁶ combine with the atoms to which they are attached to form a ring, wherein the ring is selected from 3- to 10-membered cycloalkyl and 3- to 10-membered heterocycloalkyl; the 3- to 10-membered cycloalkyl optionally substituted by at least one of halogen C₁₋₆ alkyl, C₁₋₆ haloalkyl, C₁₋₆ alkoxyl, C₁₋₆ haloalkoxyl, C₁₋₆ hydroxyalkyl, C₁₋₆ alkoxyC₁₋₆alkyl, or oxo; the 3- to 10-membered heterocycloalkyl optionally substituted by at least one of halogen, hydroxyl, C₁₋₆ alkyl, C₁₋₆ alkoxyl, C₁₋₆ haloalkyl, C₁₋₆ haloalkoxyl, —NR^{D}R^{E}, C₁₋₆alkoxyC₁₋₆alkyl, C₁₋₆haloalkoxyC₁₋₆alkyl, C₆₋₁₀aryl, C₅₋₁₀ heteroaryl, C(O)C₁₋₆alkyl, C(O)OC₁₋₆ alky, or oxo;
in the 4 to 10-membered ring, the 5-6 membered heterocycloalkylene, 5-6 membered heteroarylene, heterocycloalkyl, and 3- to 10-membered heterocycloalkyl, the heteroatom is independently selected from one or more of N, O and S, and the number of the heteroatom is independently 1-3;
scheme 2:
A is 5-6 membered heterocycloalkylene, phenylene or phenylene optionally substituted by hydroxy or boric acid group;
G is C₁₋₆ alkylene;
X^{a} is CH;
X^{b} is P or B;
Y is N;
W is absent, O or S, provided that when X^{b} is B, W is absent;
R¹ and R² are each independently selected from hydroxy and C₁₋₆ alkyl,
R³ is C₁₋₆alkoxy substituted by C₁₋₆ alkyl;
R⁴ is hydrogen or C₁₋₆ alky;
E is a bond, or selected from N, or —CR^{A}—, wherein R^{A} is selected from hydrogen, halogen, hydroxy or C₁₋₆ alkyl;
R⁵ is hydrogen, C₁₋₆ alkyl, 3- to 10-membered heterocycloalkyl, C₃₋₁₀ cycloalkyl, C₆₋₁₀ aryl, C₅₋₁₀ heteroaryl, or 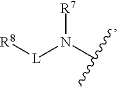
wherein R⁷ is hydrogen, or C₁₋₆ alkyl; R⁸ is C₁₋₆ alkyl, 3- to 10-membered heterocycloalkyl; the 3- to 10-membered heterocycloalkyl optionally substituted by C₁₋₆ alkyl, C₆₋₁₀ aryl, —CO—O—C₃₋₆ cycloalkyl or —CO—O—C₁₋₆ alkyl; the C₃₋₁₀ cycloalkyl and C₃₋₆ cycloalkyl optionally substituted by hydroxyl, cyano, halogen, or C₁₋₆ alkyl;
L is absent, or selected from —CH₂—, —C(O)—, —CHR⁹— or —C(R⁹)₂—, wherein R⁹ is C₁₋₆ alkyl;
R⁶ is hydrogen, C₁₋₆ alkyl or C₃₋₆ cycloalkyl;
or R^{S} and R⁶ combine with the atoms to which they are attached to form a ring, wherein the ring is selected from 3- to 10-membered cycloalkyl and 3- to 10-membered heterocycloalkyl; the 3- to 10-membered cycloalkyl optionally substituted by at least one of halogen C₁₋₆ alkyl, C₁₋₆ haloalkyl, C₁₋₆ alkoxyl, C₁₋₆ haloalkoxyl, C₁₋₆ hydroxyalkyl, C₁₋₆ alkoxyC₁₋₆alkyl, or oxo; the 3- to 10-membered heterocycloalkyl optionally substituted by at least one of halogen, hydroxyl, C₁₋₆ alkyl, C₁₋₆ alkoxyl, C₁₋₆ haloalkyl, C₁₋₆ haloalkoxyl, —NR^{D}R^{E}, C₁₋₆alkoxyC₁₋₆alkyl, C₁₋₆haloalkoxyC₁₋₆alkyl, C₆₋₁₀aryl, C₅₋₁₀heteroaryl, C(O)C₁₋₆alkyl, C(O)OC₁₋₆ alky, or oxo;
in 3- to 10-membered heterocycloalkyl, the heteroatom is independently selected from one or two of N and O, and the number of the heteroatom is independently 1-2;
in 3- to 6-membered heterocycloalkyl, the heteroatom is independently selected from one or two of N and 0, and the number of the heteroatom is independently 1-2;
in 5-6 membered heterocycloalkylene, the heteroatom is independently selected from one or two of N and S, and the number of the heteroatom is independently 1-2;
scheme 3:
wherein A is 5-6 heterocycloalkylene, phenylene or phenylene optionally substituted by hydroxy or boric acid group;
G is C₁₋₆ alkylene;
X^{a} is CH;
X^{b} is P or B;
Y is N;
W is absent, O or S, provided that when X^{b} is B, W is absent;
R¹ and R² are each independently selected from hydroxy and C₁₋₆ alkyl,
R³ is C₁₋₆ alkoxy substituted by C₁₋₆ alkyl;
R⁴ is hydrogen or C₁₋₆ alky;
E is a bond, or selected from N, or —CR^{A}—, wherein R^{A} is selected from hydrogen, halogen, hydroxy or C₁₋₆ alkyl;
R⁵ is hydrogen, C₁₋₆ alkyl, or 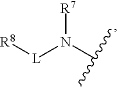
wherein R⁷ is hydrogen, or C₁₋₆ alkyl;
R⁸ is C₁₋₆ alkyl, 3- to 6-membered heterocycloalkyl; the 3- to 6-membered heterocycloalkyl optionally substituted by C₆₋₁₀ to aryl or —CO—O—C₁₋₆alkyl;
L is absent, or —C(O)—,
R⁶ is hydrogen, C₁₋₆ alkyl or C₃₋₆ cycloalkyl;
in 3- to 6-membered heterocycloalkyl, the heteroatom is independently selected from one or two of N and 0, and the number of the heteroatom is independently 1-2;
in 5-6 heterocycloalkylene, the heteroatom is independently selected from one or two of N and O, and the number of the heteroatom is independently 1-2.

8. the compound of formula (I), or the pharmaceutically acceptable salt thereof, the solvate thereof or the solvate of the pharmaceutically acceptable salt according to claim 1, wherein, the compound of formula (I) meets one or more of the following conditions: (1) A is 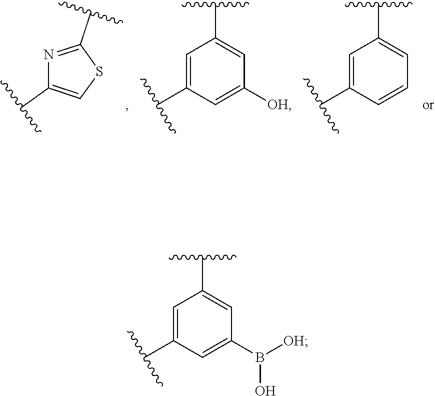 [SMILES: *c1csc(*)n1 ; *c1cc(*)cc(O)c1 ; *c1cccc(*)c1 ; *c1cc(*)cc(B(O)O)c1]
(2) G is 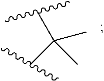 [SMILES: *C(*)(C)C]
(3) 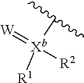
is 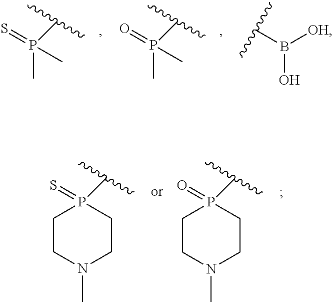
(4) R³ is 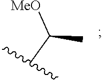 [SMILES: *[C@H](C)OC]
(5) R⁴ is 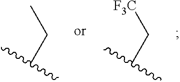
(6) 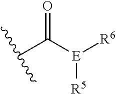
is

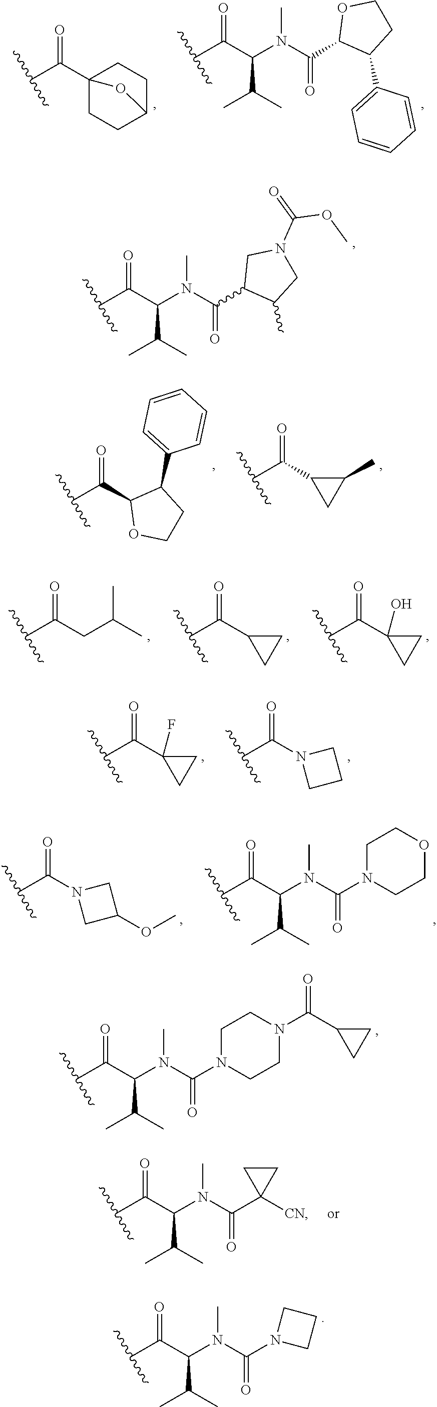

9. the compound of formula (1), or the pharmaceutically acceptable salt thereof, the solvate thereof or the solvate of the pharmaceutically acceptable salt according to claim 1, wherein, the compound of formula (I) is selected from any one of the following compounds:

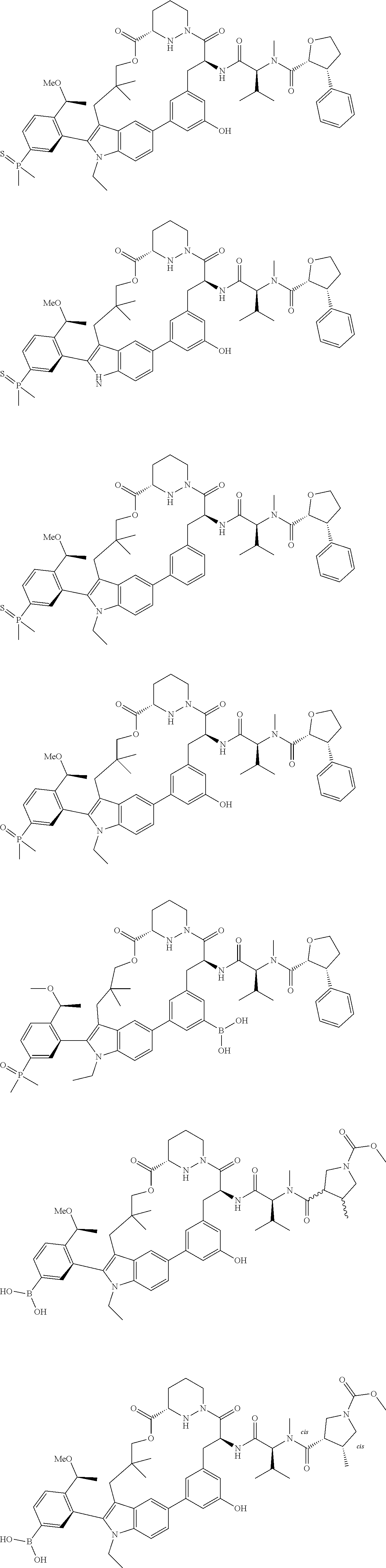

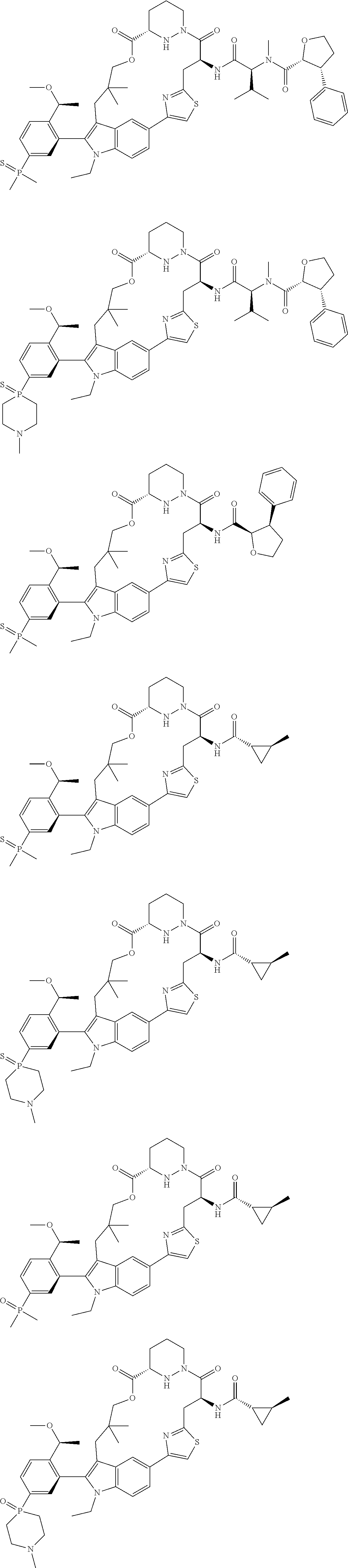

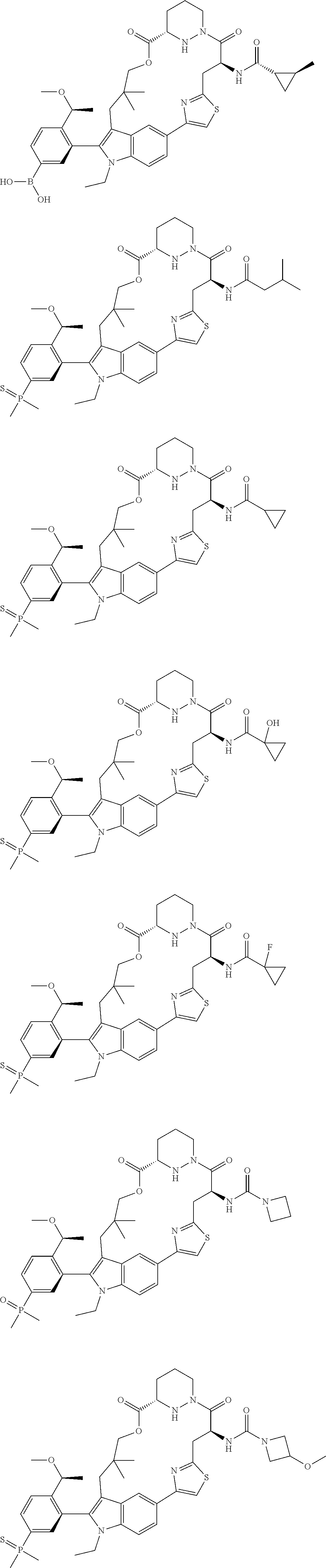

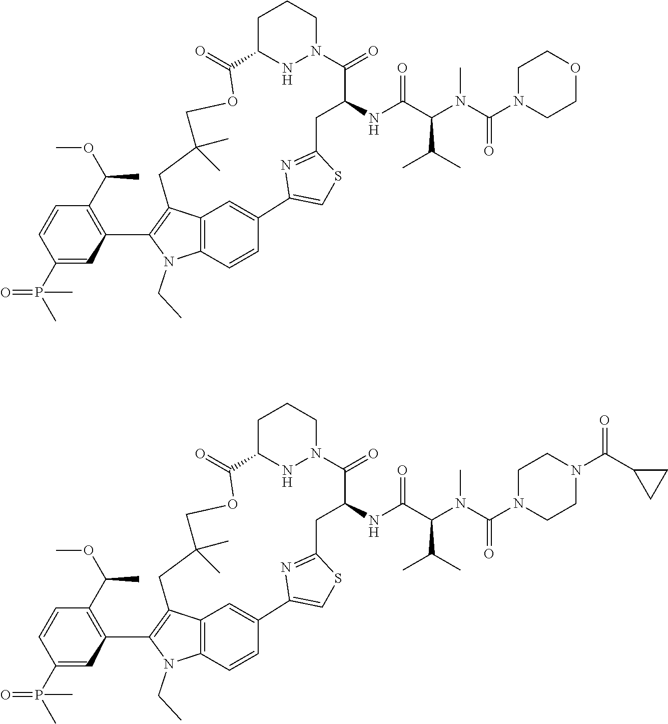

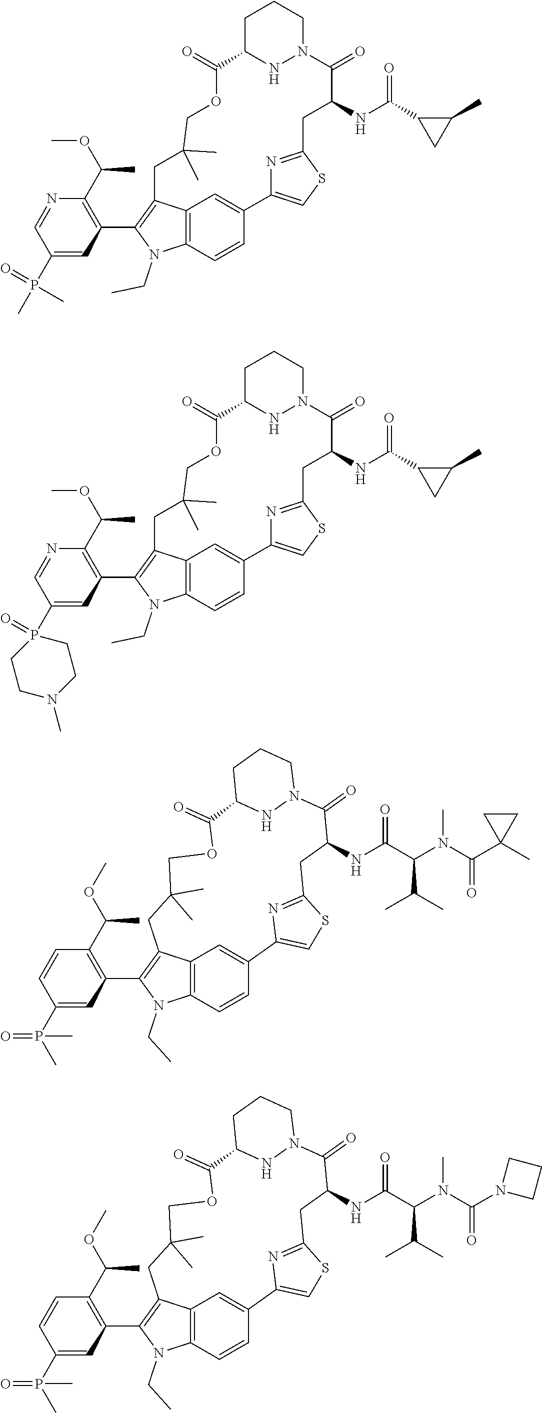

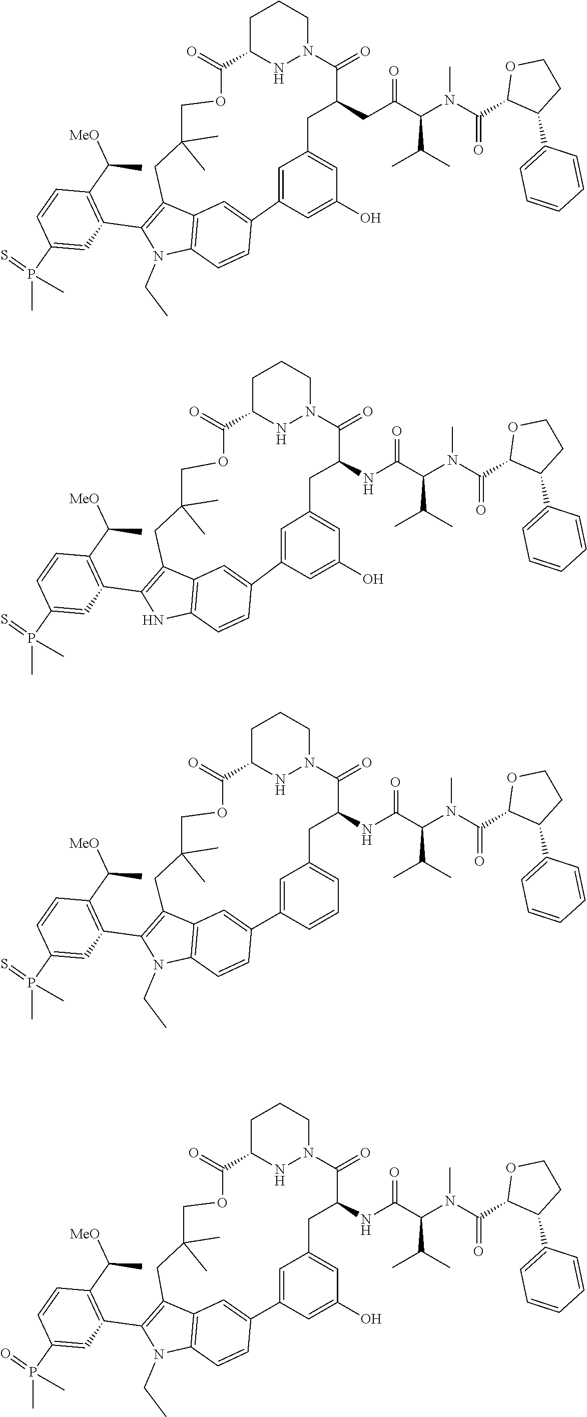

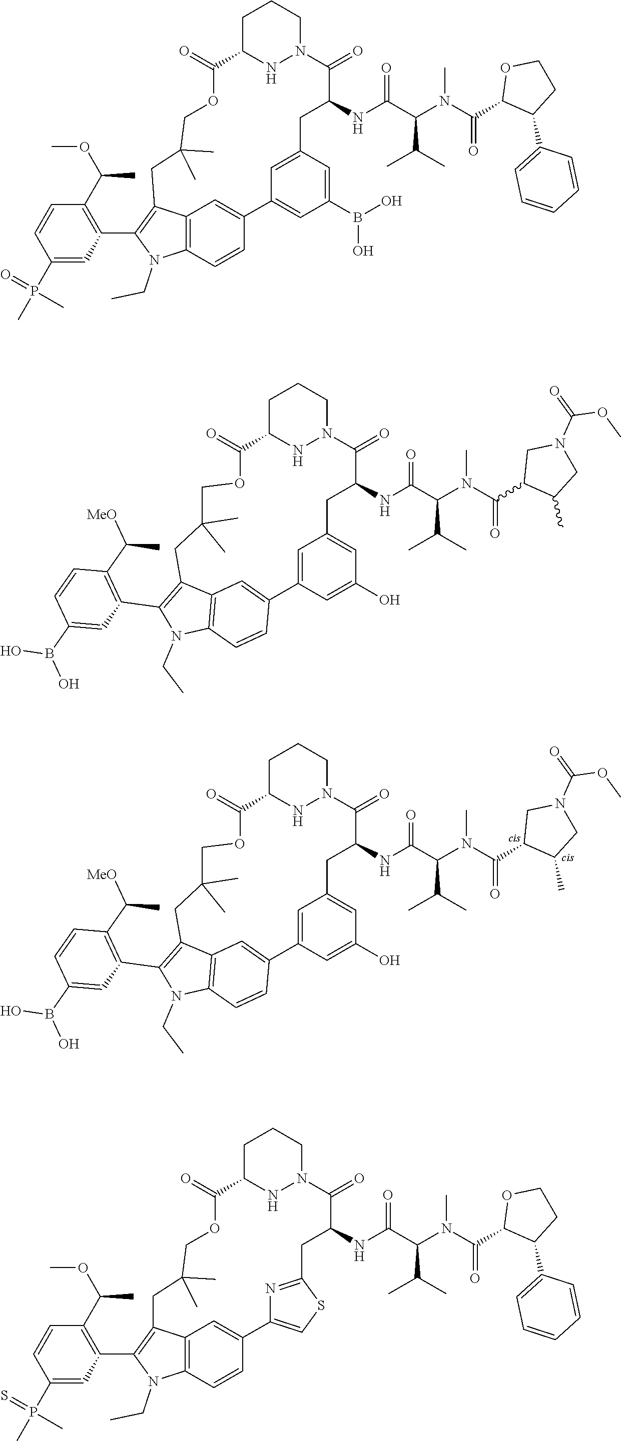

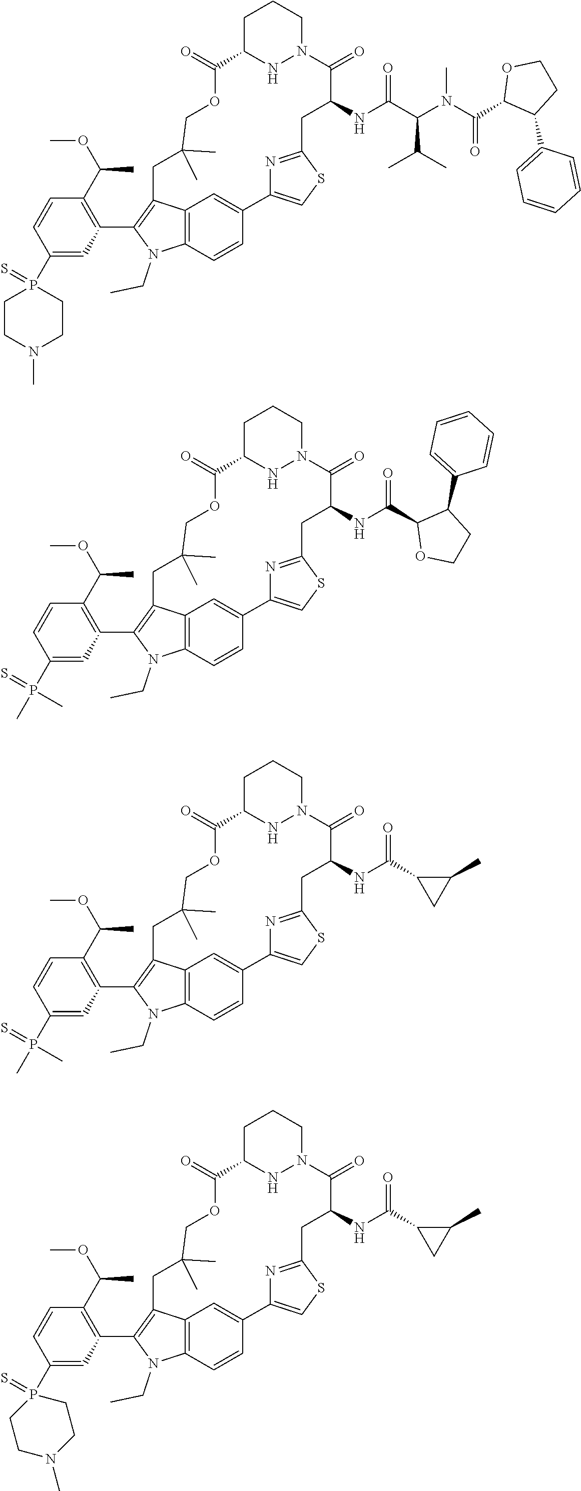

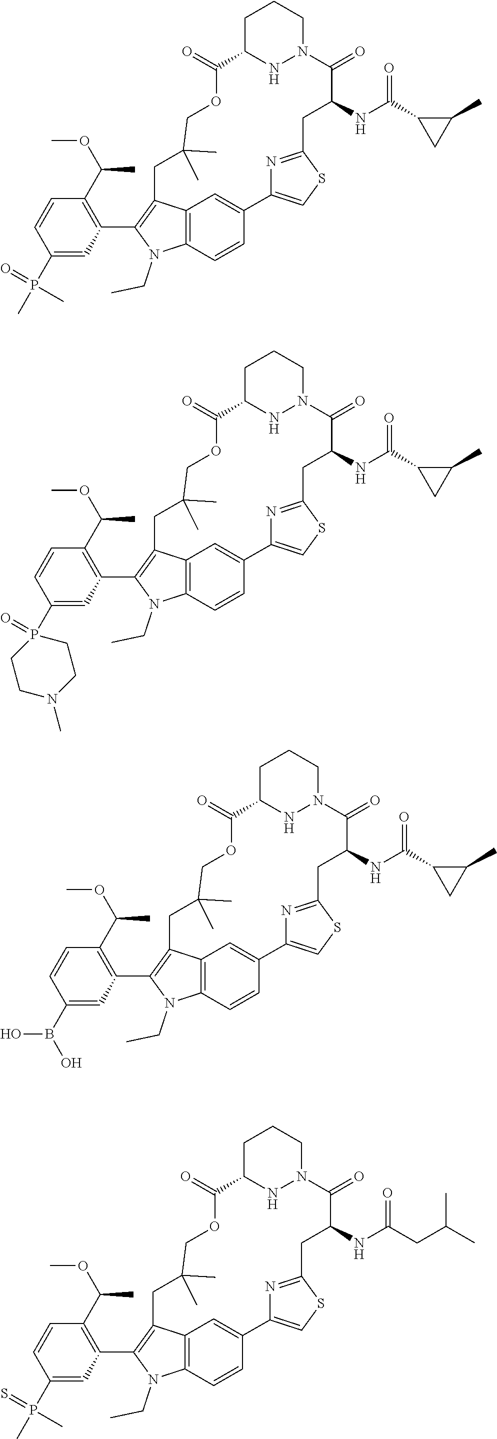

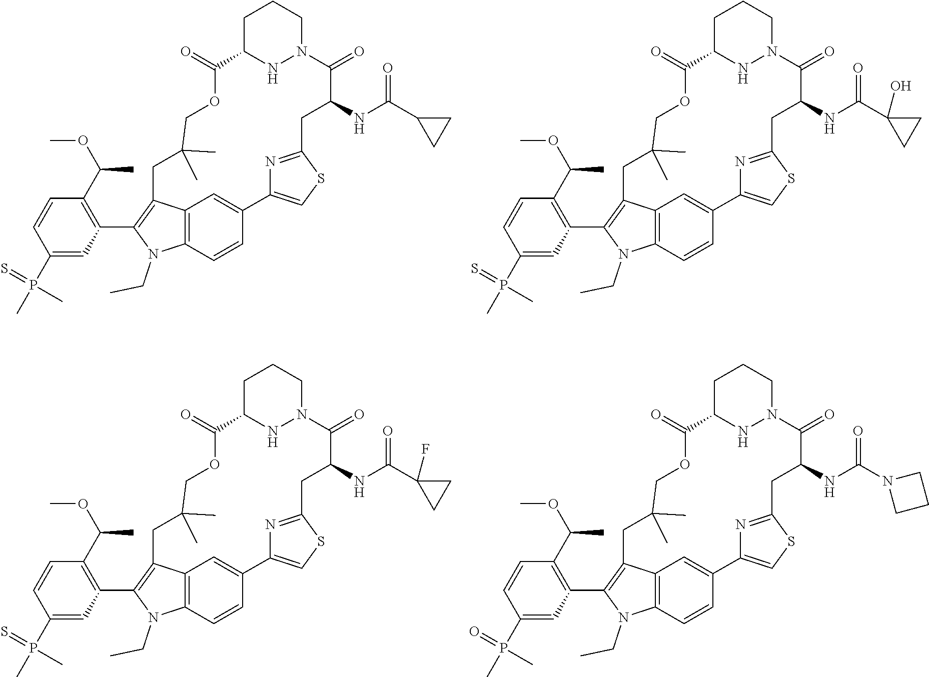

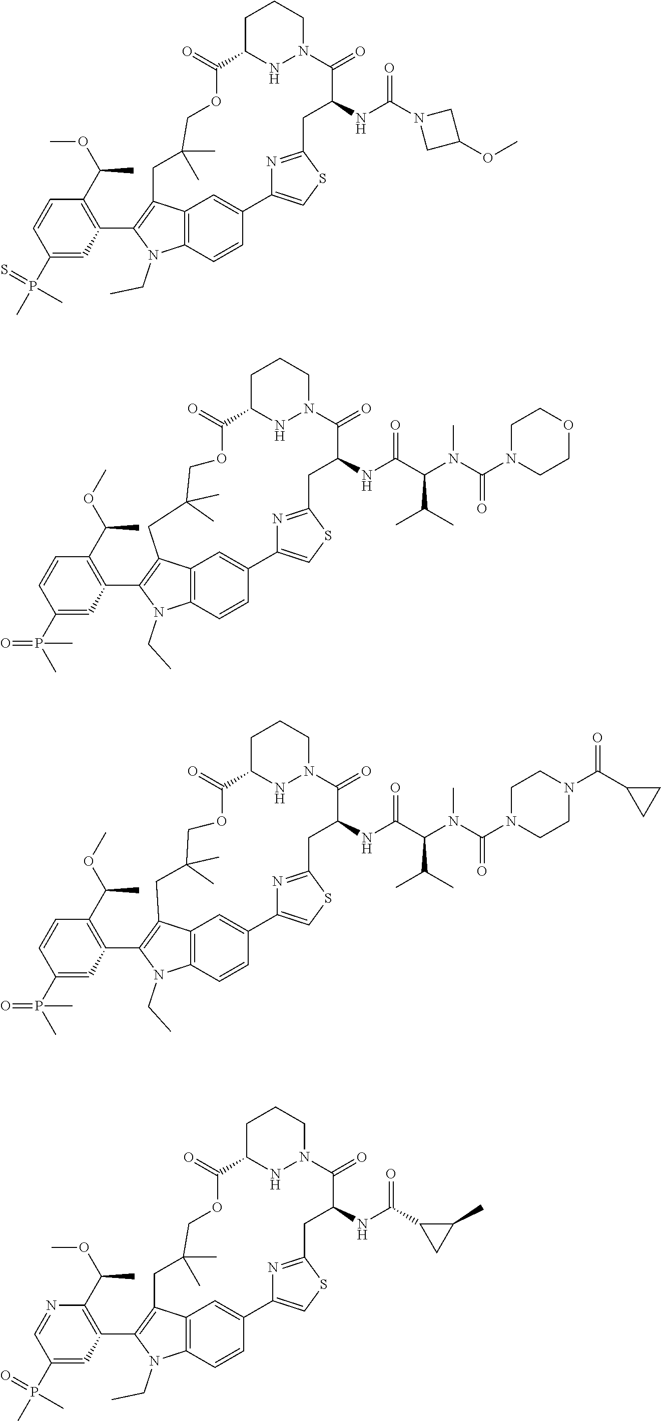

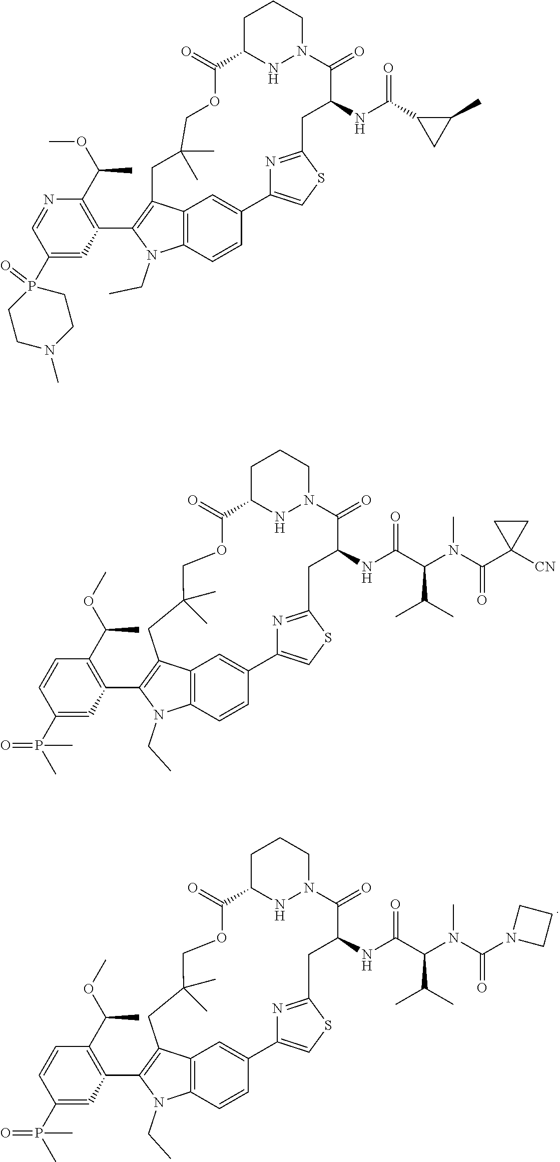

10. A pharmaceutical composition comprising a compound, wherein a pharmaceutically acceptable salt thereof, a solvate thereof or a solvate of the pharmaceutically acceptable salt, and a pharmaceutically acceptable carrier, wherein the compound is the compound of formula (I) according to any one of claims 1-9.

11. A use of a compound, a pharmaceutically acceptable salt thereof, a solvate thereof or a solvate of the pharmaceutically acceptable salt, or the pharmaceutical composition according to claim 10 in the preparation of a medicament used for the treatment of cancer, wherein the compound is the compound of formula (I) according to any one of claims 1-9.

12. The use according to claim 11, wherein the cancer is a Ras-driven cancer.

13. The use according to claim 11, wherein the cancer comprises a Ras mutation.

14. The use according to claim 13, wherein the Ras mutation is a position 12, 13 or 61.

15. The use according to claim 13, wherein the Ras mutation is at position 12.

16. The use according to claim 13, wherein the Ras mutation is at a position selected from the group consisting of G12C, G12D, G12V, G12A, G12R, G12S, G13C, G13D, Q61H, Q61R and Q61L, or a combination thereof.

17. The use according to claim 16, wherein the Ras mutation is a position selected from the group consisting of G12D, G12V, and G12R, or a combination thereof.

18. The use according to any one of claimsll-17, wherein the cancer is pancreatic cancer, appendiceal cancer, small bowel cancer, colorectal cancer, ampullary cancer, non-small cell lung cancer, cervical cancer, lung cancer, endometrial cancer, acute myeloid leukemia, gastrointestinal neuroendocrine tumour, uterine endometrioid carcinoma, oesophagogastric cancer, bladder cancer, ovarian cancer, melanoma, multiple myeloma, thyroid gland adenocarcinoma, a myelodysplastic syndrome, or squamous cell lung carcinoma.

19. The use according to claim 18, wherein the cancer is pancreatic cancer, lung cancer, or colorectal cancer.

20. The use according to any one of claims 11-19, wherein the Ras protein is KRAS.

21. A method of treating cancer in a subject in need thereof, the method comprising administering to the subject a therapeutically effective amount of the compound, the pharmaceutically acceptable salt thereof, the solvate thereof or the solvate of the pharmaceutically acceptable salt, or the pharmaceutical composition according to claim 10, wherein the compound is the compound of formula (1) according to any one of claims 1-9.

22. The method according to claim 21, wherein the cancer is a Ras-driven cancer.

23. The method according to claim 21, wherein the cancer comprises a Ras mutation.

24. The method according to claim 23, wherein the Ras mutation is a position 12, 13 or 61.

25. The method according to claim 24, wherein the Ras mutation is at position 12.

26. The method according to any one of claims 23-25, wherein the Ras mutation is at a position selected from the group consisting of G12C, G12D, G12V, G12A, G12R, G12S, G13C, G13D, Q61H, Q61R and Q61L, or a combination thereof.

27. The method according to claim 26, wherein the Ras mutation is a position selected from the group consisting of G12D, G12V, and G12R, or a combination thereof.

28. The method according to any one of claims 21-27, wherein the cancer is pancreatic cancer, appendiceal cancer, small bowel cancer, colorectal cancer, ampullary cancer, non-small cell lung cancer, cervical cancer, lung cancer, endometrial cancer, acute myeloid leukemia, gastrointestinal neuroendocrine tumour, uterine endometrioid carcinoma, oesophagogastric cancer, bladder cancer, ovarian cancer, melanoma, multiple myeloma, thyroid gland adenocarcinoma, a myelodysplastic syndrome, or squamous cell lung carcinoma.

29. The method according to claim 28, wherein the cancer is pancreatic cancer, lung cancer, or colorectal cancer.

30. The method according to any one of claims 21-29, wherein the Ras protein is KRAS.

31. The method according to any one of claims 21-30, wherein the method is provided of treating a Ras protein-related disorder in a subject in need thereof.

32. A compound, wherein the compound is selected from any one of the following compounds: 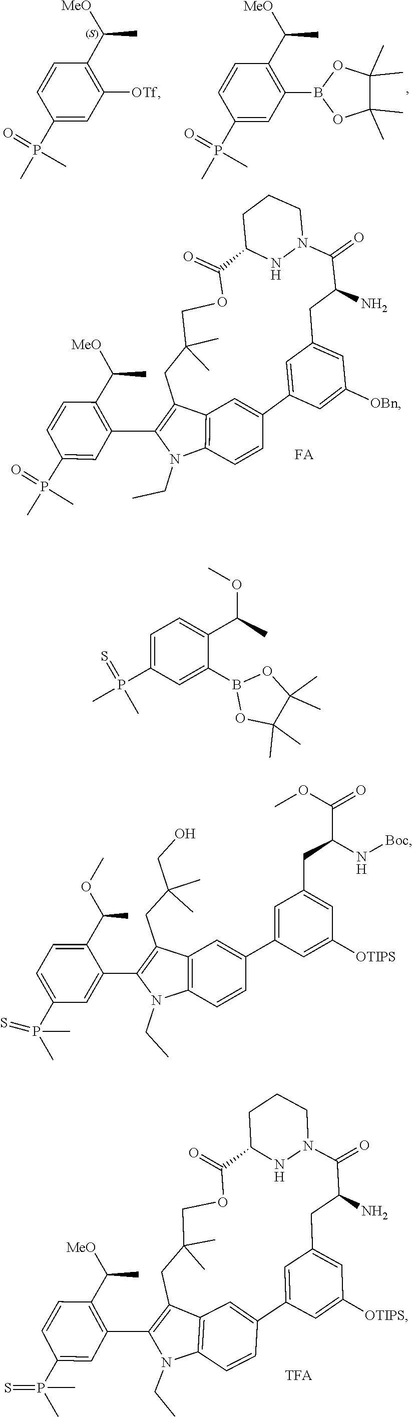 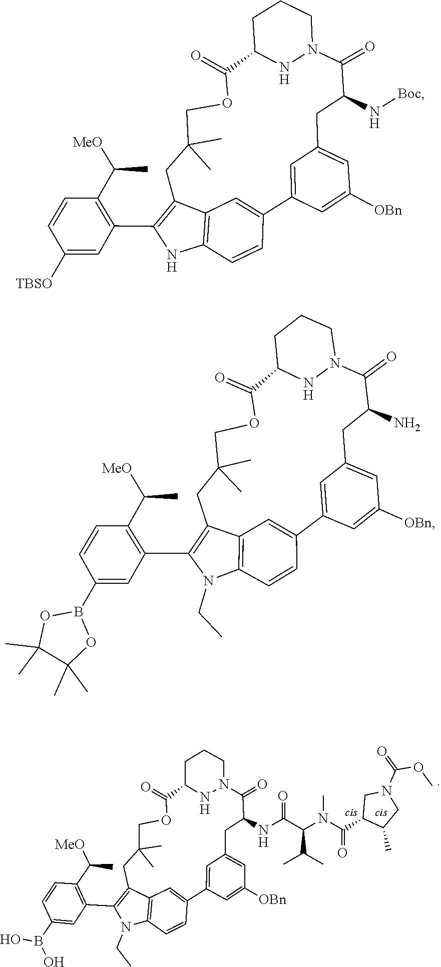 [SMILES: CO[C@@H](C)c1ccc(O[Si](C)(C)C(C)(C)C)cc1-c1[nH]c2ccc3cc2c1CC(C)(C)COC(=O)[C@@H]1CCCN(N1)C(=O)[C@@H](NC(=O)OC(C)(C)C)Cc1cc(OCc2ccccc2)cc-3c1 ; CCn1c(-c2cc(B3OC(C)(C)C(C)(C)O3)ccc2[C@H](C)OC)c2c3cc(ccc31)-c1cc(cc(OCc3ccccc3)c1)C[C@H](N)C(=O)N1CCC[C@H](N1)C(=O)OCC(C)(C)C2 ; CCn1c(-c2cc(B(O)O)ccc2[C@H](C)OC)c2c3cc(ccc31)-c1cc(cc(OCc3ccccc3)c1)C[C@H](NC(=O)[C@H](C(C)C)N(C)C(=O)[C@@H]1CN(C(=O)OC)C[C@@H]1C)C(=O)N1CCC[C@H](N1)C(=O)OCC(C)(C)C2]
